(19) United States
(12) Patent Application Publication
(10) Pub. No.: US 2009/0227556 A1
(43) Pub. Date: Sep. 10, 2009

(54) RECEPTOR TYROSINE KINASE INHIBITORS COMPRISING PYRIDINE AND PYRIMIDINE DERIVATIVES

(75) Inventor: Hiroshi OBAISHI, Tsukuba-shi (JP)

Correspondence Address: BIRCH STEWART KOLASCH & BIRCH, PO BOX 747, FALLS CHURCH, VA, 22040-0747, US

(73) Assignee: Eisai R&D Management Co., Ltd., Tokyo (JP)

(21) Appl. No.: 12/359,475

(22) Filed: Jan. 26, 2009

(30) Foreign Application Priority Data
Jan. 31, 2008 (JP) 2008-021195

(51) Int. Cl. A61K 31/4427; A61K 31/496; A61K 31/4545; A61P 35/04

(52) U.S. Cl. 514/210.2; 514/253.01; 514/318

(57) ABSTRACT

A compound represented by the following formula, a salt thereof or a hydrate of the foregoing can inhibit VEGFR-1, VEGFR-2, VEGFR-3, RON, RET and/or KIT.

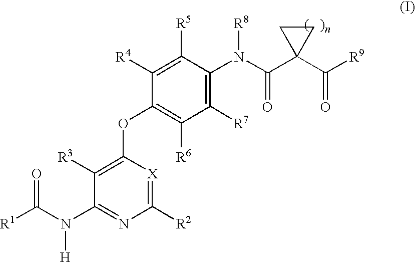

[R¹ represents a 3- to 10-membered non-aromatic heterocyclic group or the like; R² and R³ represent hydrogen; R⁴, R⁵, R⁶, and R⁷ may be the same or different and each represents hydrogen, halogen, C₁₋₆ alkyl or the like; R⁸ represents hydrogen or the like; R⁹ represents a 3- to 10-membered non-aromatic heterocyclic group or the like; n represents an integer of 1 or 2; X represents —CH═, nitrogen or the like.]

### RELATED APPLICATIONS

This application claims priority to Japanese Patent Application 2008-21195 filed on Jan. 31, 2008, which is herein incorporated by reference by in its entirety.

### BACKGROUND OF THE INVENTION

1. Field of the Invention

The present invention relates to inhibitors comprising a pyridine and pyrimidine derivative, against kinase activity of VEGFR-1 (hereinafter also referred to as “FLT1”), VEGFR-2 (hereinafter also referred to as “KDR”), VEGFR-3 (hereinafter also referred to as “FLT4”), RON, RET and KIT. The present invention also relates to methods of treating a disease associated with activation of VEGFR-1, VEGFR-2, VEGFR-3, RON, RET and/or KIT, comprising administering a pyridine and pyrimidine derivative to a patient in need thereof.

2. Related Background Art

Intracellular signaling through a receptor tyrosine kinase contributes to cell proliferation, differentiation, and metabolism, and as a result, it causes various diseases such as cancer (Non-Patent Documents 1 and 2). The receptor tyrosine kinase includes VEGFR-1, VEGFR-2, VEGFR-3, RON, RET, KIT, and the like.

Kit

It is known that binding of KIT to SCF (Stem cell factor), a ligand specific for KIT, causes KIT's dimerization and subsequently activates kinase activity, resulting in phosphorylation of various KIT substrates which exist in a cell (Non-Patent Documents 3 and 4).

It is considered that abnormal activation of KIT becomes a proliferative signal in certain kinds of cancer cells (examples are described below) and causes canceration and malignant transformation.

(1) Acute myelogenous leukemia, AML: KIT expression was observed in many (60-80%) patients with AML, and proliferation of blast cells derived from those patients was promoted by SCF stimulation. In addition, KIT activation was observed without SCF stimulation in 13 out of 18 patients, indicating that activating mutation of KIT has likely occurred in those patients (Non-Patent Documents 4-8).
(2) Mast cell leukemia: The presence of activating mutation of KIT has been reported in cell lines of mast cell leukemia developed in mastocytosis patients (Non-Patent Document 9).
(3) Small cell lung carcinoma, SCLC: Highly expressed KIT was observed in 70% or more of SCLC cell lines. On the other hand, in non-small cell lung carcinoma cell lines, the expression of KIT was a little or below the detection limit. Also, in the SCLC cell lines, SCF, a ligand, was also expressed, suggesting that proliferation was possibly promoted through autocrine (Non-Patent Documents 10 and 11).
(4) Gastrointestinal stromal tumors, GIST: GIST is defined as stromal cancer developed in the gastrointestinal tract expressing KIT. Activating mutation is observed in about a half of GIST patients and exists more frequently in highly malignant GIST suggesting the possibility that it is a prognostic factor (Non-Patent Documents 12 and 13).
(5) Testicular cancer: In testicular cancer, carcinoma in situ (CIS), which is considered to be a precancerous lesion, develops into tumors referred as seminoma and non-seminoma. KIT has been reported to be highly expressed in CIS and seminoma (Non-Patent Document 14). Recently, it has been further reported that KIT which has undergone activating mutation has been expressed in seminoma (Non-Patent Document 15).
(6) Ovarian cancer: It has been reported that only SCF but no KIT is expressed in the normal ovarian epithelium, although both KIT and SCF are expressed in benign ovarian cancer in its early stages of malignant transformation; and in further malignantly transformed ovarian cancer, the expression of KIT is reduced. These findings suggest that KIT plays an important role in the development of ovarian cancer (Non-Patent Document 16).
(7) Breast cancer: In breast cancer, the KIT expression has been reportedly decreased as compared to the normal surrounding tissue (Non-Patent Document 17), however, in the subsequent study, in breast cancer, the KIT expression which had not been observed in the normal tissue was observed, and SCF expression was further observed, suggesting that the proliferation was promoted by autocrine stimulation (Non-Patent Document 18).
(8) Brain cancer: It is reported that KIT expression was observed in cell lines and tissues of glioblastoma which is the most malignant among brain cancer; and SCF stimulation promoted proliferation in KIT-expressing cell lines of glioblastoma (Non-Patent Document 19).
(9) Neuroblastoma: It has been reported that in cell lines and tissue samples of neuroblastoma, which is well-known as a cancer developed in children, SCF and KIT are often expressed together, and that the proliferation of the neuroblastoma cell line is promoted by autocrine because it is inhibited by anti-KIT antibody (Non-Patent Document 20).
(10) Colorectal cancer: In colorectal cancer tissue, co-expression of KIT and its ligand, SCF was observed, while in the normal mucosal tissue, none of their expression was observed. Additionally, proliferation of the colorectal cancer cell line was promoted by SCF stimulation (Non-Patent Document 21).

Moreover, SCF-stimulated KIT activation has been reported to be essential for the proliferation and differentiation of mast cells (Non-Patent Documents 22 and 23). Thus, overactivation of KIT is considered to cause such immune abnormalities as mastocytosis, asthma, and chronic rhinitis which are induced by excessive mast cells.

(1) Mastocytosis: It is a generic term for various pathological conditions characterized by mast cell hyperproliferation (Non-Patent Documents 24 and 25). In mastocytosis patients, 1) overexpression of KIT (Non-Patent Document 26), 2) increased amount of soluble SCF (Non-Patent Document 27), 3) activating mutation of KIT (Non-Patent Documents 26 and 28), and the like have been reported, and those conditions are likely to overactivate KIT to cause mastocytosis.
(2) Allergy, Asthma: Mast cells and eosinophils are important cells in developing inflammation, allergy, asthma and the like (Non-Patent Documents 29 and 30). This fact is also suggested by reports that a corticosteroid, which is considered to be most effective for chronic rhinitis and allergy-induced inflammation, decreases the number of circulating and infiltrating mast cells and eosinophils (Non-Patent Documents 31 and 32). It has been reported that the SCF-stimulated KIT activation is not only essential for differentiation, survival, and proliferation of the mast cells, but it promotes induction of various factors from the mast cells, and that those factors carry out important functions in differentiation, survival, and infiltration of eosinophils (Non-Patent Documents 33-38). Therefore, it is thought that inhibition of KIT is likely to be able to inhibit activated mast cells and eosinophils in patients with asthma, allergy and the like. VEGFR-1, VEGFR-2, and VEGFR-3

Neovascularization is a biological phenomenon essential for vascular tree formation, as well as morphological and functional development of each organ in the fetal period. It has been reported that new blood vessels are constructed through multiple processes, such as migration, proliferation, and lumen formation of endothelial cells, and that involvement of mast cells, lymphocytes, and interstitial cells is essential for these processes (Non-Patent Document 39).

More than one angiogenesis stimulating factors in the body have been identified, among which vascular endothelial growth factor has been reported to promote neovascularization (Non-Patent Document 40).

In matured individuals, new blood vessels are formed physiologically in wound healing or in female estrus cycle, while it has been known that abnormally increased neovascularization in matured individuals relates to development or progression processes of various diseases. Specifically, diseases which involve abnormal neovascularization include cancer, rheumatoid arthritis, atherosclerosis, diabetic retinopathy, angioma, psoriasis, and the like (Non-Patent Document 41). Proliferation of solid cancer, in particular, reportedly depends on the neovascularization, and anti-angiogenic agents are expected to become a new therapeutic drug for refractory solid cancers (Non-Patent Document 42).

Furthermore, relationships have been indicated between VEGF and the following diseases. VEGF sometimes causes inflammation-related tissue edema (Non-Patent Document 43). In many human tumor-cell lines including glioblastoma multiforme, hemangioblastoma, central nervous system tumor, and AIDS-related Kaposi's sarcoma, VEGF is demonstrated to be highly expressed (Non-Patent Documents 44-47).

As receptors for VEGF, VEGFR-1, VEGFR-2, and VEGFR-3 have been identified. These receptors are involved in neovascularization and participate in signal transduction (Non-Patent Document 48). Therefore, an agent which inhibits VEGFR-1, VEGFR-2, and VEGFR-3 is considered to be effective as a therapeutic agent for diseases associated with neovascularization and VEGF.

RON

A short transcription product of RON (mutated RON) which is found in patients with lung cancer, ovarian cancer, and gastrointestinal stromal cancer has reportedly induced the promotion of anchorage-dependent and anchorage-independent proliferation in human breast cancer cell line (T47D) (Non-Patent Document 49).

It has been reported that anti-RON antibody (IMC-41A10), a RON kinase inhibitor, exhibited an anti-tumor effect in models subcutaneously implanted with human colorectal cancer cell line (HT29), human lung cancer cell line (NCI-H292), and human pancreas cancer cell line (BxPC-3) which highly express non-mutated RON (Non-Patent Document 50).

Thus, it is suggested that the RON kinase inhibitor exhibits a cell growth inhibition or anti-tumor effect on cells expressing mutated and non-mutated form of RON. Also, the RON kinase inhibitor is thought to be effective for diseases caused by RON.

### RET

Mutation of RET is reported to have induced anchorage-independent growth and tumorigenicity in NIH3T3 cells (Non-Patent Document 51).

Furthermore, it has been reported that ZD6474, a RET kinase inhibitor, suppressed anchorage-independent growth in the NIH3T3 cells transformed by mutated RET and inhibited tumorigenesis after the injection of the cells to a nude mouse (Non-Patent Document 51).

Furthermore, a report has said that in a model subcutaneously implanted with human thyroid cancer cell line (TT), BAY 43-9006, a RET kinase inhibitor, reduced a tumor size (Non-Patent Document 52).

Thus, it is suggested that the RET kinase inhibitor causes inhibition of cell proliferation of cells expressing mutated RET, and exhibits the anti-tumor effect on tumors including the above mentioned cells. Thus, the RET kinase inhibitor is thought to be effective for diseases caused by the mutation of the RET.

Here, as a compound having a HGFR kinase inhibitory activity, a compound represented by the following formula (I) has been known (Patent Document 1).

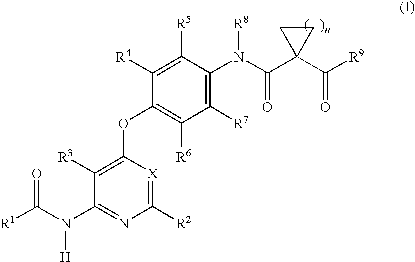

wherein, R¹ represents a 3-10 membered nonaromatic heterocyclic ring or the like. R² and R³ represent a hydrogen atom. R⁴, R⁵, R⁶, and R⁷ are the same or different and represent a hydrogen atom, a halogen atom, a C₁₋₆ alkyl group or the like. R⁸ represents a hydrogen atom or the like. R⁹ represents a 3-10 membered nonaromatic heterocyclic group or the like. n represents an integer from 1 to 2. X represents a group represented by a formula of —CH═ or a nitrogen atom.

However, it has never been reported that the above mentioned compound has KDR, VEGFR-1, VEGFR-3, RON, RET, and KIT kinase inhibitory activities.
[Patent Document 1] WO 2007/023768
[Non-Patent Document 1] Kolibaba et al., B. B. A., 1333:F217-F248, 1997
[Non-Patent Document 2] Sheijen et al., Oncogene, 21:3314-3333, 2002
[Non-Patent Document 3] Blume-Jensen et al., EMBO J., 10:4121-4128, 1991
[Non-Patent Document 4] Lev et al., EMBO J., 10:647-654, 1991
[Non-Patent Document 5] Wang et al., Leukemia, 3:699-702, 1989
[Non-Patent Document 6] Kanakura et al., Leuk. Lymph., 10:35-41, 1993
[Non-Patent Document 7] Ikeda et al., Blood, 78:2962-2968, 1991
[Non-Patent Document 8] Ikeda et al., Exp. Hematol., 21:1686-1694, 1993
[Non-Patent Document 9] Furitsu et al., J. Clin. Invest., 92:1736-1744, 1993
[Non-Patent Document 10] Hibi et al., Oncogene, 6:2291-2296, 1991
[Non-Patent Document 11] Sekido et al., Cacer Res., 51:2416-2419, 1991
[Non-Patent Document 12] Lasota et al., Am. J. Pathol., 157:1091-1095, 2000
[Non-Patent Document 13] Taniguchi et al., Cancer Res., 59:4297-4300, 1999
[Non-Patent Document 14] Strohmeyer et al., Cancer Res., 51:1811-1816, 1991
[Non-Patent Document 15] Tian et al., Am. J. Pathol., 154:1643-1647, 1999
[Non-Patent Document 16] Tonary, Int. J. Cancer, 89:242-250, 2000
[Non-Patent Document 17] Natali et al., Int. J. Cancer, 52:713-717, 1992
[Non-Patent Document 18] Hines et al., Cell Growth & Differentiation, 6:769-779, 1995
[Non-Patent Document 19] Berdel et al., Cancer Res., 52:3498-3502, 1992
[Non-Patent Document 20] Cohen, Blood, 84:3465-3472, 1994
[Non-Patent Document 21] Bellone et al., J. Cell. Physiol., 172:1-11, 1997
[Non-Patent Document 22] Hamel et al., J. Neuro-One., 35:327-333, 1997
[Non-Patent Document 23] Kitamura et al., Int. Arch. Aller. Immunol., 107:54-56, 1995
[Non-Patent Document 24] Metcalfe, J. Invest. Derm., 93:2 S-4S, 1991
[Non-Patent Document 25] Golkar et al., Lancet, 349:1379-1385, 1997
[Non-Patent Document 26] Nagata et al., Mastocytosis Leuk., 12:175-181, 1998
[Non-Patent Document 27] Longley et al., New Engl. J. Med., 328:1302-1307, 1993
[Non-Patent Document 28] Longley et al., Nat. Gen., 12:312-314, 1996
[Non-Patent Document 29] Thomas et al., Gen. Pharmacol., 27:593-597, 1996
[Non-Patent Document 30] Metcalfe et al., Physiol. Rev., 77:1033-1079, 1997
[Non-Patent Document 31] Naclerio et al., JAMA, 278:1842-1848, 1997
[Non-Patent Document 32] Meltzer, Aller., 52:33-40, 1997
[Non-Patent Document 33] Okayama et al., Int. Arch. Aller. Immunol., 114:75-77, 1997
[Non-Patent Document 34] Okayama et al., Eur. J. Immunol., 28:708-715, 1998
[Non-Patent Document 35] Metcalf et al., Proc. Natl. Acad. Sci., 95:6408-6421, 1998
[Non-Patent Document 36] Kay et al., Int. Arch. Aller. Immunol., 113:196-199, 1997
[Non-Patent Document 37] Hogaboam et al., J. Immunol., 160:6166-6171, 1998
[Non-Patent Document 38] Luckas et al., J. Immunol., 156, 3945-3951, 1996
[Non-Patent Document 39] Folkman et al., J. Biol. Chem., 267:10931-10934, 1992
[Non-Patent Document 40] Jakeman et al., Endocrinology, 133:848-859, 1993
[Non-Patent Document 41] Folkman et al., New Engl. J. Med., 333:1757-1763, 1995
[Non-Patent Document 42] Folkman et al., J. Natl. Cancer Inst., 82:4-6, 1990
[Non-Patent Document 43] Ferrara et al., Endocr. Rev., 13:18, 1992
[Non-Patent Document 44] Plate et al., Nature, 359:845-848, 1992
[Non-Patent Document 45] Plate et al., Cancer Res., 53:5822-5827, 1993
[Non-Patent Document 46] Berkman et al., J. Clin. Invest., 91:153-159, 1993
[Non-Patent Document 47] Nakamura et al., J. Immunol., 158:4992-5001, 1997
[Non-Patent Document 48] Mustonen et al., J. Cell Biol., 129:895-898, 1995
[Non-Patent Document 49] Bardella et al., Cancer Research, 64:5154-5161, 2004
[Non-Patent Document 50] O'Toole et al., Cancer Research, 66:9162-9170, 2006
[Non-Patent Document 51] Carlomagno et al., Cancer Research, 62:7284-7290, 2002
[Non-Patent Document 52] Carlomagno et al., J. Natl. Cancer Inst., 98:326-334, 2006

### SUMMARY OF THE INVENTION

The object of the invention is to discover an inhibitor for VEGFR-1, VEGFR-2, VEGFR-3, RON, RET or KIT.

As a result of diligent studies, the inventors have discovered that a pyridine and pyrimidine derivative represented by the general formula (I) below, a salt thereof or a hydrate of the foregoing has excellent kinase inhibitory action against VEGFR-1, VEGFR-2, VEGFR-3, RON, RET or KIT, and completed the present invention.

Namely, the present invention provides [1] to [105] below:

[1] An inhibitor for VEGFR-1, VEGFR-2, VEGFR-3, RON, RET and/or KIT, comprising a compound represented by the following formula, a salt thereof or a hydrate of the foregoing:

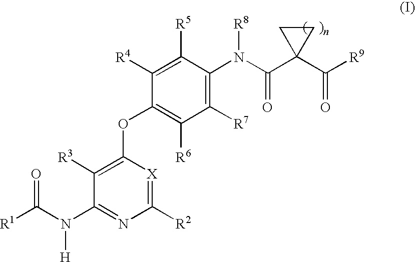

wherein R¹ represents a 3- to 10-membered non-aromatic heterocyclic group wherein the group is limited to a group having nitrogen as a ring constituent atom and the nitrogen having a bonding hand, or a group represented by the formula —NR^{11a}R^{11b}, wherein R^{11a} and R^{11b} may be the same or different and each represents hydrogen, C₁₋₆ alkyl, C₃₋₆ alkenyl, C₃₋₆ alkynyl, C₃₋₁₀ cycloalkyl, C₆₋₁₀ aryl, 5- to 10-membered heteroaryl or a 4- to 10-membered non-aromatic heterocyclic group, and R^{11a} and R^{11b} may be substituted with a substituent selected from Substituent Group A or Substituent Group B and R¹ may be substituted with a substituent selected from Substituent Group A or Substituent Group B;

R² and R³ represent hydrogen;

R⁴, R⁵, R⁶ and R⁷ may be the same or different and each represents hydrogen, halogen, hydroxyl, cyano, trifluoromethyl, C₁₋₆ alkyl, C₂₋₆ alkenyl, C₂₋₆ alkynyl, C₁₋₆ alkoxy, amino, mono-C₁₋₆ alkylamino, di-C₁₋₆ alkylamino or a group represented by the formula —CO—R¹², wherein R¹² represents hydrogen, hydroxyl, C₁₋₆ alkyl, C₁₋₆ alkoxy, amino, mono-C₁₋₆ alkylamino or di-C₁₋₆ alkylamino;

R⁸ represents hydrogen or C₁₋₆ alkyl;

R⁹ represents a 3- to 10-membered non-aromatic heterocyclic group wherein the group is limited to a group having nitrogen as a ring constituent atom and the nitrogen having a bonding hand, or a group represented by the formula —NR^{11a}R^{11b}, wherein R^{11a} and R^{11b} represent the same meaning as described above and R⁹ may be substituted with a substituent selected from Substituent Group A or Substituent Group B;

n represents an integer of 1 or 2; and

X represents a group represented by the formula —C(R¹⁰)═ or nitrogen, wherein R¹⁰ represents hydrogen, halogen, cyano, C₁₋₆ alkyl, C₂₋₆ alkenyl, C₂₋₆ alkynyl or a group represented by the formula —CO—R¹² wherein R¹² represents the same meaning as recited above;

wherein Substituent Group A consists of halogen, hydroxyl, mercapto, nitro, cyano and oxo;

wherein Substituent Group B consists of C₁₋₆ alkyl, C₂₋₆ alkenyl, C₂₋₆ alkynyl, C₃₋₁₀ cycloalkyl, C₆₋₁₀ aryl, 5- to 10-membered heteroaryl, a 3- to 10-membered non-aromatic heterocyclic group, C₁₋₆ alkoxy, C₃₋₆ alkenyloxy, C₃₋₆ alkynyloxy, C₃₋₁₀ cycloalkoxy, C₆₋₁₀ aryloxy, 5- to 10-membered heteroaryloxy, 4- to 10-membered non-aromatic heterocyclicoxy, C₁₋₆ alkylthio, C₃₋₆ alkenylthio, C₃₋₆ alkynylthio, C₃₋₁₀ cycloalkylthio, C₆₋₁₀ arylthio, 5- to 10-membered heteroarylthio, 4- to 10-membered non-aromatic heterocyclicthio and a group represented by the formula -T¹-T²-T³, and each group in Substituent Group B may be substituted with a substituent selected from Substituent Group C, wherein T¹ represents a direct bond or C₁₋₆ alkylene, T² represents carbonyl, sulfinyl, sulfonyl, a group represented by the formula —C(═O)—O—, a group represented by the formula —O—C(═O)—, a group represented by the formula —SO₂—O—, a group represented by the formula —O—SO₂—, a group represented by the formula —NR^{T1}—, a group represented by the formula —C(═O)—NR^{T1}—, a group represented by the formula —NR^{T1}—C(═O)—, a group represented by the formula —SO₂—NR^{T1}— or a group represented by the formula —NR^{T1}—SO₂—, T³ represents hydrogen, C₁₋₆ alkyl, C₃₋₆ alkenyl, C₃₋₆ alkynyl, C₃₋₁₀ cycloalkyl, C₆₋₁₀ aryl, 5- to 10-membered heteroaryl or a 4- to 10-membered non-aromatic heterocyclic group, and R^{T1} represents hydrogen or C₁₋₆ alkyl; and

wherein Substituent Group C consists of halogen, hydroxyl, mercapto, nitro, cyano, oxo, C₁₋₆ alkyl, C₂₋₆ alkenyl, C₂₋₆ alkynyl, C₃₋₁₀ cycloalkyl, C₆₋₁₀ aryl, 5- to 10-membered heteroaryl, a 3- to 10-membered non-aromatic heterocyclic group, C₁₋₆ alkoxy, C₁₋₆ alkylthio, mono-C₁₋₆ alkylamino and di-C₁₋₆ alkylamino.

[2] An inhibitor of [1], wherein R¹ represents a 3- to 10-membered non-aromatic heterocyclic group optionally substituted with a substituent selected from Substituent Group A or Substituent Group B recited in [1], wherein the group is limited to a group having nitrogen as a ring constituent atom and the nitrogen having a bonding hand.
[3] An inhibitor of [1], wherein R¹ represents a group represented by the formula (II):

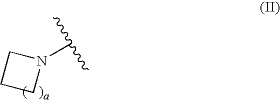

wherein a represents an integer of 1 to 4;
or a group represented by the formula (III):

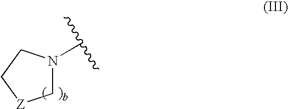

wherein b represents an integer of 1 to 3, and Z represents oxygen, sulfur, carbonyl, sulfonyl, or a group represented by the formula —NR^{Z}, wherein R^{Z} represents hydrogen or C₁₋₆ alkyl, and the groups represented by the formula (II) or (III) may be substituted with a substituent selected from Substituent Group A or Substituent Group B recited in [1].
[4] An inhibitor of [1], wherein R¹ represents azetidin-1-yl optionally substituted with a substituent selected from Substituent Group D, pyrrolidin-1-yl optionally substituted with a substituent selected from Substituent Group D, piperidin-1-yl optionally substituted with a substituent selected from Substituent Group D, azepan-1-yl optionally substituted with a substituent selected from Substituent Group D, piperazin-1-yl optionally substituted with a substituent selected from Substituent Group D, diazepan-1-yl optionally substituted with a substituent selected from Substituent Group D, morpholin-4-yl optionally substituted with a substituent selected from Substituent Group D, thiomorpholin-4-yl optionally substituted with a substituent selected from Substituent Group D, 1,1-dioxothiomorpholin-4-yl optionally substituted with a substituent selected from Substituent Group D,

wherein Substituent Group D consists of halogen, hydroxyl, mercapto, cyano, formyl, oxo, C₁₋₆ alkyl, C₃₋₁₀ cycloalkyl, C₁₋₆ alkoxy, amino, mono-C₁₋₆ alkylamino, di-C₁₋₆ alkylamino, azetidinyl, pyrrolidinyl, piperidinyl, piperazinyl, diazepanyl and a group represented by -T⁴-T⁵,

wherein T⁴ represents carbonyl or sulfonyl, and T⁵ represents C₁₋₆ alkyl, C₃₋₁₀ cycloalkyl, azetidinyl, pyrrolidinyl, piperidinyl, hydroxyl, C₁₋₆ alkoxy, amino, mono-C₁₋₆ alkylamino or di-C₁₋₆ alkylamino,

where each group included in Substituent Group D may be substituted with hydroxyl, C₁₋₆ alkyl, di-C₁₋₆ alkylamino, azetidinyl or pyrrolidinyl.

[5] An inhibitor of [1], wherein R¹ represent azetidin-1-yl optionally substituted with a substituent selected from Substituent Group E, pyrrolidin-1-yl optionally substituted with a substituent selected from Substituent Group E, piperidin-1-yl optionally substituted with a substituent selected from Substituent Group E, piperazin-1-yl optionally substituted with a substituent selected from Substituent Group E, diazepan-1-yl optionally substituted with a substituent selected from Substituent Group E or morpholin-4-yl optionally substituted with a substituent selected from Substituent Group E,

wherein Substituent Group E consists of methyl, ethyl, dimethylamino, azetidinyl, pyrrolidinyl, piperidinyl and piperazinyl,

where each group included in Substituent Group E may be substituted with hydroxyl, methyl, dimethylamino, azetidinyl, pyrrolidinyl or piperidinyl.

[6] An inhibitor of [1], wherein R¹ represents azetidin-1-yl optionally substituted with a substituent selected from Substituent Group G, pyrrolidin-1-yl optionally substituted with a substituent selected from Substituent Group G, piperidin-1-yl optionally substituted with a substituent selected from Substituent Group G or piperazin-1-yl optionally substituted with a substituent selected from Substituent Group G,

wherein Substituent Group G consists of dimethylamino, azetidinyl, pyrrolidinyl, piperidinyl, piperazinyl, dimethylaminomethyl, dimethylaminoethyl, azetidin-1-ylmethyl, pyrrolidin-1-ylmethyl and piperidin-1-ylmethyl,

where each group included in Substituent Group G may be substituted with methyl or dimethylamino.

[6-1] An inhibitor of [1], wherein R¹ represents azetidin-1-yl optionally substituted with a substituent selected from Substituent Group G-1, pyrrolidin-1-yl optionally substituted with a substituent selected from Substituent Group G-1, piperidin-1-yl optionally substituted with a substituent selected from Substituent Group G-1 or piperazin-1-yl optionally substituted with a substituent selected from Substituent Group G-1,

wherein Substituent Group G-1 consists of azetidinyl, pyrrolidinyl, piperidinyl, piperazinyl, dimethylaminomethyl, dimethylaminoethyl, azetidin-1-ylmethyl, pyrrolidin-1-ylmethyl and piperidin-1-ylmethyl,

where each group included in Substituent Group G-1 may be substituted with methyl or dimethylamino.

[6-2] An inhibitor of [1], wherein R¹ represents azetidin-1-yl having dimethylamino, pyrrolidin-1-yl having dimethylamino or piperidin-1-yl having dimethylamino.
[6-3] An inhibitor of [1], wherein R¹ represents azetidin-1-yl optionally substituted with a substituent selected from Substituent Group G-2, pyrrolidin-1-yl substituted with a substituent selected from Substituent Group G-2 or piperidin-1-yl substituted with a substituent selected from Substituent Group G-2,

wherein Substituent Group G-2 consists of hydroxyl, methoxy, hydroxymethyl and dimethylaminoacetoxy.

[6-4] An inhibitor of [1], wherein R¹ represents [2-(dimethylamino)ethyl]piperazin-1-yl, 4-pyrrolidin-1-ylpiperidin-1-yl, 4-[(dimethylamino)methyl]piperidin-1-yl, 4-azetidin-1-ylpiperidin-1-yl, 4-[3-(dimethylamino)azetidin-1-yl]piperidin-1-yl, 4-(4-methylpiperazin-1-yl)piperidin-1-yl, 4-(1-methylpiperidin-4-yl)piperazin-1-yl, 4-(1-methylazetidin-3-yl)piperazin-1-yl, 4-(dimethylamino)piperidin-1-yl, 4-(azetidin-1-ylmethyl)piperidin-1-yl, 4-(pyrrolidin-1-ylmethyl)piperidin-1-yl, (3S)-3-(dimethylamino)pyrrolidin-1-yl, (3R)-3-(dimethylamino)pyrrolidin-1-yl, azetidin-1-yl, pyrrolidin-1-yl, morpholin-4-yl, 4-methylpiperazin-1-yl, 3-hydroxyazetidin-1-yl, 1,3′-biazetidin-1′-yl, 3-(hydroxymethyl)azetidin-1-yl, 3-(dimethylamino)azetidin-1-yl, 3-[(dimethylamino)methyl] azetidin-1-yl, 4-hydroxypiperidin-1-yl, 4-(hydroxymethyl)piperidin-1-yl, (3R)-3-hydroxypyrrolidin-1-yl, (3S)-3-hydroxypyrrolidin-1-yl, 3-(azetidin-1-ylmethyl)azetidin-1-yl or 3-(2-dimethylaminoacetoxy)azetidin-1-yl.
[7] An inhibitor of [1], wherein R¹ represents a group represented by the formula —NR^{11a}R^{11b}, wherein R^{11a} and R^{11b} represent the same meaning as recited in [1].
[8] An inhibitor of [1], wherein R¹ represents a group represented by the formula —NR^{11c}R^{11d}, wherein R^{11c} represents hydrogen or C₁₋₆ alkyl, and R^{11d} represents C₁₋₆ alkyl or a group represented by the formula (IV):

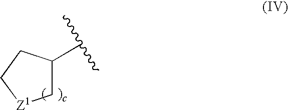

wherein c represents an integer of 1 to 3, and Z¹ represents oxygen, sulfur, carbonyl, sulfonyl or a group represented by the formula —NR^{z1}—, wherein R^{Z1} represents hydrogen or C₁₋₆ alkyl, and R^{11d} may be substituted with a substituent selected from Substituent Group A or Substituent Group B recited in [1].
[9] An inhibitor of [1], wherein R¹ represents a group represented by the formula —NR^{11e}R^{11f}, wherein R^{11e} represents hydrogen or C₁₋₆ alkyl, and R^{11f} represents C₁₋₆ alkyl, pyrrolidin-3-yl, piperidin-3-yl, piperidin-4-yl or tetrahydropyran-4-yl, and R^{11f} if may be substituted with a substituent selected from Substituent Group D recited in [4].
[10] An inhibitor of [1], wherein R¹ represents a group represented by the formula —NR^{11g}R^{11h}, wherein R^{11g} represents hydrogen or methyl, and R^{11h} represents n-propyl, n-butyl, pyrrolidin-3-yl, piperidin-3-yl, piperidin-4-yl or tetrahydropyran-4-yl, and R^{11h} may be substituted with a substituent selected from Substituent Group F,

wherein Substituent Group F consists of methyl, ethyl, n-propyl, acetyl, dimethylamino, diethylamino, azetidinyl, pyrrolidinyl and piperazinyl,

where each group included in Substituent Group F may be substituted with methyl or dimethylamino.

[11] An inhibitor of [1], wherein R¹ represents a group represented by the formula —N(CH₃)R¹¹ⁱ, wherein R¹¹ⁱ represents n-propyl, n-butyl, pyrrolidin-3-yl or piperidin-4-yl, and R¹¹ⁱ may be substituted with a substituent selected from Substituent Group H,

wherein Substituent Group H consists of dimethylamino, diethylamino, dimethylaminoethyl, dimethylaminopropyl and 1-methylazetidin-3-yl.

[12] An inhibitor of [1], wherein R¹ represents a group represented by the formula —N(CH₃)R^{11j}, wherein R^{11j} represents 1-methylpiperidin-4-yl or 1-ethylpiperidin-4-yl.
[12-1] An inhibitor of [1], wherein R¹ represents a group represented by the formula —N(CH₃)R^{11k}, wherein R^{11k} represents 3-(dimethylamino)propyl or 1-[2-(dimethylamino)ethyl]piperidin-4-yl.
[12-2] An inhibitor of [1], wherein R¹ represents methyl(1-methylpiperidin-4-yl)amino, (1-ethylpiperidin-4-yl)(methyl)amino, [3-(dimethylamino)propyl] (methyl)amino or {1-[2-(dimethylamino) ethyl]piperidin-4-yl}(methyl)amino.
[13] An inhibitor of any one of [1] to [12-2], wherein R⁴, R⁵, R⁶ and R⁷ may be the same or different and each represents hydrogen, halogen or C₁₋₆ alkyl.
[14] An inhibitor of any one of [1] to [13], wherein R⁸ represents hydrogen.
[15] An inhibitor of any one of [1] to [14], wherein X represents a group represented by the formula —C(R^{10a})=, wherein R^{10a} represents hydrogen, halogen or cyano.
[16] An inhibitor of any one of [1] to [14], wherein X represents nitrogen.
[17] An inhibitor of any one of [1] to [16], wherein n represents 1.
[18] An inhibitor of any one of [1] to [17], wherein R⁹ represents mono-C₁₋₆ alkylamino optionally substituted with a substituent selected from Substituent Group A or Substituent Group B recited in [1], mono-C₃₋₁₀ cycloalkylamino optionally substituted with a substituent selected from Substituent Group A or Substituent Group B recited in [1], mono-C₆₋₁₀ arylamino optionally substituted with a substituent selected from Substituent Group A or Substituent Group B recited in [1], mono-5- to 10-membered heteroarylamino optionally substituted with a substituent selected from Substituent Group A or Substituent Group B recited in [1] or mono-4- to 10-membered non-aromatic heterocyclic amino optionally substituted with a substituent selected from Substituent Group A or Substituent Group B recited in [1].
[19] An inhibitor of any one of [1] to [17], wherein R⁹ represents mono-C₃₋₁₀ cycloalkylamino optionally substituted with a substituent selected from Substituent Group A or Substituent Group B recited in [1] or mono-C₆₋₁₀ arylamino optionally substituted with a substituent selected from Substituent Group A or Substituent Group B recited in [1].
[19-1] An inhibitor of any one of [1] to [17], wherein R⁹ represents mono-C₃₋₁₀ cycloalkylamino optionally substituted with a substituent selected from Substituent Group I or mono-C₆₋₁₀ arylamino optionally substituted with a substituent selected from Substituent Group I,

wherein Substituent Group I consists of halogen, trifluoromethyl, cyano, C₁₋₆ alkyl and C₁₋₆ alkoxy.

[19-2] An inhibitor of any one of [1] to [17], wherein R⁹ represents cyclopentylamino optionally substituted with a substituent selected from Substituent Group I recited in [19-1], cyclohexylamino optionally substituted with a substituent selected from Substituent Group I recited in [19-1], cycloheptylamino optionally substituted with a substituent selected from Substituent Group I recited in [19-1] or phenylamino optionally substituted with a substituent selected from Substituent Group I recited in [19-1].
[20] An inhibitor of [1], wherein a compound represented by the formula (I) is
(1) N-[4-({2-[({4-[2-(Dimethylamino)ethyl]piperazin-1-yl}carbonyl)amino]pyridin-4-yl}oxy)-2-fluorophenyl]-N′-(4-fluorophenyl)cyclopropane-1,1-dicarboxamide,
(2) N-(2-Fluoro-4-{[2-({[methyl(1-methylpiperidin-4-yl)amino]carbonyl}amino)pyridin-4-yl]oxy}phenyl)-N′-(4-fluorophenyl)cyclopropane-1,1-dicarboxamide,
(3) N-(4-Fluorophenyl)-N′-{2-fluoro-4-[(2-{[(4-pyrrolidin-1-ylpiperidin-1-yl)carbonyl]amino}pyridin-4-yl)oxy]phenyl}cyclopropane-1,1-dicarboxamide,
(4) N-[4-({2-[({4-[(Dimethylamino)methyl]piperidin-1-yl}carbonyl)amino]pyridin-4-yl}oxy)-2-fluorophenyl]-N′-(4-fluorophenyl)cyclopropane-1,1-dicarboxamide,
(5) N-{4-[(2-{[(4-Azetidin-1-ylpiperidin-1-yl)carbonyl]amino}pyridin-4-yl)oxy]-2-fluorophenyl}-N′-(4-fluorophenyl)cyclopropane-1,1-dicarboxamide,
(6) N-[4-({2-[({4-[3-(Dimethylamino)azetidin-1-yl]piperidin-1-yl}carbonyl)amino]pyridin-4-yl}oxy)-2-fluorophenyl]-N′-(4-fluorophenyl)cyclopropane-1,1-dicarboxamide,
(7) N-(2-Fluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide,
(8) N-(2-Fluoro-4-{[2-({[4-(1-methylpiperidin-4-yl)piperazin-1-yl]carbonyl}amino)pyridin-4-yl]oxy}phenyl)-N′-(4-fluorophenyl)cyclopropane-1,1-dicarboxamide,
(9) N-(2-Fluoro-4-{[2-({[4-(1-methylazetidin-3-yl)piperazin-1-yl]carbonyl}amino)pyridin-4-yl]oxy}phenyl)-N′-(4-fluorophenyl)cyclopropane-1,1-dicarboxamide,
(10) N-(4-{[2-({[4-(Dimethylamino)piperidin-1-yl]carbonyl}amino)pyridin-4-yl]oxy}-2-fluorophenyl)-N′-(4-fluorophenyl)cyclopropane-1,1-dicarboxamide,
(11) N-(4-{[2-({[4-(Azetidin-1-ylmethyl)piperidin-1-yl]carbonyl}amino)pyridin-4-yl]oxy}-2-fluorophenyl)-N′-(4-fluorophenyl)cyclopropane-1,1-dicarboxamide,
(12) N-(4-Fluorophenyl)-N′-(2-fluoro-4-{[2-({[4-(pyrrolidin-1-ylmethyl)piperidin-1-yl]carbonyl}amino)pyridin-4-yl]oxy}phenyl)cyclopropane-1,1-dicarboxamide,
(13) N-(4-{[2-({[(3S)-3-(Dimethylamino)pyrrolidin-1-yl]carbonyl}amino)pyridin-4-yl]oxy}-2-fluorophenyl)-N′-(4-fluorophenyl)cyclopropane-1,1-dicarboxamide,
(14) N-(4-{[2-({[(3R)-3-(Dimethylamino)pyrrolidin-1-yl]carbonyl}amino)pyridin-4-yl]oxy}-2-fluorophenyl)-N′-(4-fluorophenyl)cyclopropane-1,1-dicarboxamide,
(15) N-(2-Fluoro-4-{[2-({[methyl(1-methylpiperidin-4-yl)amino]carbonyl}amino)pyridin-4-yl]oxy}phenyl)-N′-phenylcyclopropane-1,1-dicarboxamide,
(16) N-(2-Fluoro-4-{[2-({[4-(4-methylpiperazin-1-yl)piperidin-1-yl]carbonyl}amino)pyridin-4-yl]oxy}phenyl)-N′-phenylcyclopropane-1,1-dicarboxamide,
(17) N-[4-({2-[({4-[3-(Dimethylamino)azetidin-1-yl]piperidin-1-yl}carbonyl)amino]pyridin-4-yl}oxy)-2-fluorophenyl]-N′-phenylcyclopropane-1,1-dicarboxamide,
(18) N-(4-{[2-({[(1-Ethylpiperidin-4-yl)(methyl)amino]carbonyl}amino)pyridin-4-yl]oxy}-2-fluorophenyl)-N′-phenylcyclopropane-1,1-dicarboxamide,
(19) N-[4-({2-[(Azetidin-1-ylcarbonyl)amino]pyridin-4-yl}oxy)-2-fluorophenyl]-N′-(4-fluorophenyl)cyclopropane-1,1-dicarboxamide,
(20) N-(4-Fluorophenyl)-N′-[2-fluoro-4-({2-[(pyrrolidin-1-ylcarbonyl)amino]pyridin-4-yl}oxy)phenyl]cyclopropane-1,1-dicarboxamide,
(21) N-{2-Fluoro-4-[(2-{[(3-hydroxyazetidin-1-yl)carbonyl]amino}pyridin-4-yl)oxy]phenyl}-N′-(4-fluorophenyl)cyclopropane-1,1-dicarboxamide,
(22) N-[4-({2-[(1,3′-Biazetidin-1′-ylcarbonyl)amino]pyridin-4-yl}oxy)-2-fluorophenyl]-N′-(4-fluorophenyl)cyclopropane-1,1-dicarboxamide,
(23) N-(2-Fluoro-4-{[2-({[3-(hydroxymethyl)azetidin-1-yl]carbonyl}amino)pyridin-4-yl]oxy}phenyl)-N′-(4-fluorophenyl)cyclopropane-1,1-dicarboxamide,
(24) N-(4-{[2-({[3-(Dimethylamino)azetidin-1-yl]carbonyl}amino)pyridin-4-yl]oxy}-2-fluorophenyl)-N′-(4-fluorophenyl)cyclopropane-1,1-dicarboxamide,
(25) N-[4-({2-[({3-[(Dimethylamino)methyl]azetidin-1-yl}carbonyl)amino]pyridin-4-yl}oxy)-2-fluorophenyl]-N′-(4-fluorophenyl)cyclopropane-1,1-dicarboxamide,
(26) N-{2-Fluoro-4-[(2-{[(4-hydroxypiperidin-1-yl)carbonyl]amino}pyridin-4-yl)oxy]phenyl}-N′-(4-fluorophenyl)cyclopropane-1,1-dicarboxamide,
(27) N-(2-Fluoro-4-{[2-({[4-(hydroxymethyl)piperidin-1-yl]carbonyl}amino)pyridin-4-yl]oxy}phenyl)-N′-(4-fluorophenyl)cyclopropane-1,1-dicarboxamide,
(28) N-(2-Fluoro-4-{[2-({[(3R)-3-hydroxypyrrolidin-1-yl]carbonyl}amino)pyridin-4-yl]oxy}phenyl)-N′-(4-fluorophenyl)cyclopropane-1,1-dicarboxamide,
(29) N-(2-Fluoro-4-{[2-({[(3S)-3-hydroxypyrrolidin-1-yl]carbonyl}amino)pyridin-4-yl]oxy}phenyl)-N′-(4-fluorophenyl)cyclopropane-1,1-dicarboxamide,
(30) N-[4-({2-[(Azetidin-1-ylcarbonyl)amino]pyridin-4-yl}oxy)-2,5-difluorophenyl]-N′-(4-fluorophenyl)cyclopropane-1,1-dicarboxamide,
(31) N-{2,5-Difluoro-4-[(2-{[(3-hydroxyazetidin-1-yl)carbonyl]amino}pyridin-4-yl)oxy]phenyl}-N′-(4-fluorophenyl)cyclopropane-1,1-dicarboxamide,
(32) N-(2,5-Difluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide,
(33) N-[2,5-Difluoro-4-({2-[({3-[(dimethylamino)methyl] azetidin-1-yl}carbonyl)amino]pyridin-4-yl}oxy)phenyl]-N′-(4-fluorophenyl)cyclopropane-1,1-dicarboxamide,
(34) N-(2,5-Difluoro-4-{[2-({[methyl(1-methylpiperidin-4-yl)amino]carbonyl}amino)pyridin-4-yl]oxy}phenyl)-N′-(4-fluorophenyl)cyclopropane-1,1-dicarboxamide,
(35) N-{4-[(2-{[3-(Azetidin-1-ylmethyl)azetidin-1-ylcarbonyl]amino}pyridin-4-yl)oxy]-2,5-difluorophenyl}-N′-(4-fluorophenyl)cyclopropane-1,1-dicarboxamide,
(36) N-(2,5-Difluoro-4-{[2-({[3-(hydroxymethyl)azetidin-1-yl]carbonyl}amino)pyridin-4-yl]oxy}phenyl)-N′-(4-fluorophenyl)cyclopropane-1,1-dicarboxamide,
(37) N-{2,5-Difluoro-4-[(4-{[(3-hydroxyazetidin-1-yl)carbonyl]amino}pyrimidin-6-yl)oxy]phenyl}-N′-(4-fluorophenyl)cyclopropane-1,1-dicarboxamide,
(38) N-[4-({4-[({3-[(Dimethylamino)methyl] azetidin-1-yl}carbonyl)amino]pyrimidin-6-yl}oxy)-2,5-difluorophenyl]-N′-(4-fluorophenyl)cyclopropane-1,1-dicarboxamide,
(39) N-(2,5-Difluoro-4-{[4-({[3-(hydroxymethyl)azetidin-1-yl]carbonyl}amino)pyrimidin-6-yl]oxy}phenyl)-N′-(4-fluorophenyl)cyclopropane-1,1-dicarboxamide,
(40) N-(2,5-Difluoro-4-{[4-({[methyl(1-methylpiperidin-4-yl)amino]carbonyl}amino)pyrimidin-6-yl]oxy}phenyl)-N′-(4-fluorophenyl)cyclopropane-1,1-dicarboxamide,
(41) N-(2,5-Difluoro-4-{[4-({[4-(4-methylpiperazin-1-yl)piperidin-1-yl]carbonyl}amino)pyrimidin-6-yl]oxy}phenyl)-N′-(4-fluorophenyl)cyclopropane-1,1-dicarboxamide,
(42) N-(4-{[2-({[4-(Dimethylamino)piperidin-1-yl]carbonyl}amino)pyridin-4-yl]oxy}-2,5-difluorophenyl)-N′-(4-fluorophenyl)cyclopropane-1,1-dicarboxamide,
(43) N-{2,5-Difluoro-4-[(2-{[(4-methylpiperazin-1-yl)carbonyl]amino}pyridin-4-yl]oxy}phenyl)-N′-(4-fluorophenyl)cyclopropane-1,1-dicarboxamide,
(44) N-{2,5-Difluoro-4-[(2-{[(4-hydroxypiperidin-1-yl)carbonyl]amino}pyridin-4-yl)oxy]phenyl}-N′-(4-fluorophenyl)cyclopropane-1,1-dicarboxamide,
(45) N-{4-[(2-{[(4-Azetidin-1-ylpiperidin-1-yl)carbonyl]amino}pyridin-4-yl)oxy]oxy}-2,5-difluorophenyl}-N′-(4-fluorophenyl)cyclopropane-1,1-dicarboxamide,
(46) N-(2,5-Difluoro-4-{[2-({[3-(2-dimethylaminoacetoxy)azetidin-1-yl]carbonyl}amino)pyridin-4-yl]oxy}phenyl)-N′-(4-fluorophenyl)cyclopropane-1,1-dicarboxamide,
(47) N-(2,5-Difluoro-4-{[2-({[(3S)-3-hydroxypyrrolidin-1-yl]carbonyl}amino)pyridin-4-yl]oxy}phenyl)-N′-(4-fluorophenyl)cyclopropane-1,1-dicarboxamide or
(48) N-(2,5-Difluoro-4-{[2-({[(3R)-3-hydroxypyrrolidin-1-yl]carbonyl}amino)pyridin-4-yl]oxy}phenyl)-N′-(4-fluorophenyl)cyclopropane-1,1-dicarboxamide.
   [21] An inhibitor of [1], wherein a compound represented by the formula (I) is
(1) N-(2-Fluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide
(2) N-[4-({2-[(Azetidin-1-ylcarbonyl)amino]pyridin-4-yl}oxy)-2-fluorophenyl]-N′-(4-fluorophenyl)cyclopropane-1,1-dicarboxamide
(3) N-{2,5-Difluoro-4-[(2-{[(3-hydroxyazetidin-1-yl)carbonyl]amino}pyridin-4-yl)oxy]phenyl}-N′-(4-fluorophenyl)cyclopropane-1,1-dicarboxamide
(4) N-(2,5-Difluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide
(5) N-(2,5-Difluoro-4-{[2-({[methyl(1-methylpiperidin-4-yl)amino]carbonyl}amino)pyridin-4-yl]oxy}phenyl)-N′-(4-fluorophenyl)cyclopropane-1,1-dicarboxamide or
(6) N-(2,5-Difluoro-4-{[2-({[3-(hydroxymethyl)azetidin-1-yl]carbonyl}amino)pyridin-4-yl]oxy}phenyl)-N′-(4-fluorophenyl)cyclopropane-1,1-dicarboxamide.
   [22] A method of treating a disease associated with activation of VEGFR-1, VEGFR-2, VEGFR-3, RON, RET and/or KIT, comprising administering to a patient in need thereof, a compound represented by the following formula, a salt thereof or a hydrate of the foregoing:

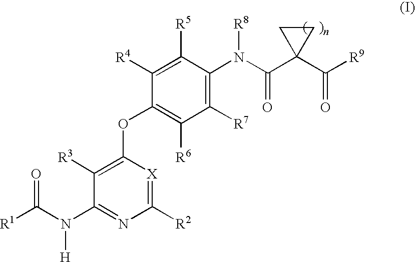

wherein R¹ represents a 3- to 10-membered non-aromatic heterocyclic group wherein the group is limited to a group having nitrogen as a ring constituent atom and the nitrogen having a bonding hand, or a group represented by the formula —NR^{11a}R^{11b}, wherein R^{11a} and R^{11b} may be the same or different and each represents hydrogen, C₁₋₆ alkyl, C₃₋₆ alkenyl, C₃₋₆ alkynyl, C₃₋₁₀ cycloalkyl, C₆₋₁₀ aryl, 5- to 10-membered heteroaryl or a 4- to 10-membered non-aromatic heterocyclic group, and R^{11a} and R^{11b} may be substituted with a substituent selected from Substituent Group A or Substituent Group B and R¹ may be substituted with a substituent selected from Substituent Group A or Substituent Group B;

R² and R³ represent hydrogen;

R⁴, R⁵, R⁶ and R⁷ may be the same or different and each represents hydrogen, halogen, hydroxyl, cyano, trifluoromethyl, C₁₋₆ alkyl, C₂₋₆ alkenyl, C₂₋₆ alkynyl, C₁₋₆ alkoxy, amino, mono-C₁₋₆ alkylamino, di-C₁₋₆ alkylamino or a group represented by the formula —CO—R¹², wherein R¹² represents hydrogen, hydroxyl, C₁₋₆ alkyl, C₁₋₆ alkoxy, amino, mono-C₁₋₆ alkylamino or di-C₁₋₆ alkylamino;

R⁸ represents hydrogen or C₁₋₆ alkyl;

R⁹ represents a 3- to 10-membered non-aromatic heterocyclic group wherein the group is limited to a group having nitrogen as a ring constituent atom and the nitrogen having a bonding hand, or a group represented by the formula —NR^{11a}R^{11b}, wherein R^{11a} and R^{11b} represent the same meaning as described above and R⁹ may be substituted with a substituent selected from Substituent Group A or Substituent Group B;

n represents an integer of 1 or 2; and

X represents a group represented by the formula —C(R¹⁰)═ or nitrogen, wherein R¹⁰ represents hydrogen, halogen, cyano, C₁₋₆ alkyl, C₂₋₆ alkenyl, C₂₋₆ alkynyl or a group represented by the formula —CO—R¹², wherein R¹² represents the same meaning as recited above;

wherein Substituent Group A consists of halogen, hydroxyl, mercapto, nitro, cyano and oxo;

wherein Substituent Group B consists of C₁₋₆ alkyl, C₂₋₆ alkenyl, C₂₋₆ alkynyl, C₃₋₁₀ cycloalkyl, C₆₋₁₀ aryl, 5- to 10-membered heteroaryl, a 3- to 10-membered non-aromatic heterocyclic group, C₁₋₆ alkoxy, C₃₋₆ alkenyloxy, C₃₋₆ alkynyloxy, C₃₋₁₀ cycloalkoxy, C₆₋₁₀ aryloxy, 5- to 10-membered heteroaryloxy, 4- to 10-membered non-aromatic heterocyclicoxy, C₁₋₆ alkylthio, C₃₋₆ alkenylthio, C₃₋₆ alkynylthio, C₃₋₁₀ cycloalkylthio, C₆₋₁₀ arylthio, 5- to 10-membered heteroarylthio, 4- to 10-membered non-aromatic heterocyclicthio and a group represented by the formula -T¹-T²-T³, and each group in Substituent Group B may be substituted with a substituent selected from Substituent Group C, wherein T¹ represents a direct bond or C₁₋₆ alkylene, T² represents carbonyl, sulfinyl, sulfonyl, a group represented by the formula —C(═O)—O—, a group represented by the formula —O—C(═O)—, a group represented by the formula —SO₂—O—, a group represented by the formula —O—SO₂—, a group represented by the formula —NR^{T1}—, a group represented by the formula —C(═O)—NR^{T1}—, a group represented by the formula —NR^{T1}—C(═O)—, a group represented by the formula —SO₂—NR^{T1}— or a group represented by the formula —NR^{T1}—SO₂—, T³ represents hydrogen, C₁₋₆ alkyl, C₃₋₆ alkenyl, C₃₋₆ alkynyl, C₃₋₁₀ cycloalkyl, C₆₋₁₀ aryl, 5- to 10-membered heteroaryl or a 4- to 10-membered non-aromatic heterocyclic group, and R^{T1} represents hydrogen or C₁₋₆ alkyl; and

wherein Substituent Group C consists of halogen, hydroxyl, mercapto, nitro, cyano, oxo, C₁₋₆ alkyl, C₂₋₆ alkenyl, C₂₋₆ alkynyl, C₃₋₁₀ cycloalkyl, C₆₋₁₀ aryl, 5- to 10-membered heteroaryl, a 3- to 10-membered non-aromatic heterocyclic group, C₁₋₆ alkoxy, C₁₋₆ alkylthio, mono-C₁₋₆ alkylamino and di-C₁₋₆ alkylamino.

[23] A method of [22], wherein R¹ represents a 3- to 10-membered non-aromatic heterocyclic group optionally substituted with a substituent selected from Substituent Group A or Substituent Group B recited in [22], wherein the group is limited to a group having nitrogen as a ring constituent atom and the nitrogen having a bonding hand.
[24] A method of [22], wherein R¹ represents a group represented by the formula (II):

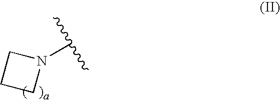

wherein a represents an integer of 1 to 4;
or a group represented by the formula (III):

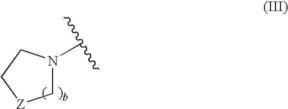

wherein b represents an integer of 1 to 3, and Z represents oxygen, sulfur, carbonyl, sulfonyl, or a group represented by the formula —NR^{Z}, wherein R^{Z} represents hydrogen or C₁₋₆ alkyl, and the groups represented by the formula (II) or (III) may be substituted with a substituent selected from Substituent Group A or Substituent Group B recited in [22].
[25] A method of [22], wherein R¹ represents azetidin-1-yl optionally substituted with a substituent selected from Substituent Group D, pyrrolidin-1-yl optionally substituted with a substituent selected from Substituent Group D, piperidin-1-yl optionally substituted with a substituent selected from Substituent Group D, azepan-1-yl optionally substituted with a substituent selected from Substituent Group D, piperazin-1-yl optionally substituted with a substituent selected from Substituent Group D, diazepan-1-yl optionally substituted with a substituent selected from Substituent Group D, morpholin-4-yl optionally substituted with a substituent selected from Substituent Group D, thiomorpholin-4-yl optionally substituted with a substituent selected from Substituent Group D, 1,1-dioxothiomorpholin-4-yl optionally substituted with a substituent selected from Substituent Group D,

wherein Substituent Group D consists of halogen, hydroxyl, mercapto, cyano, formyl, oxo, C₁₋₆ alkyl, C₃₋₁₀ cycloalkyl, C₁₋₆ alkoxy, amino, mono-C₁₋₆ alkylamino, di-C₁₋₆ alkylamino, azetidinyl, pyrrolidinyl, piperidinyl, piperazinyl, diazepanyl and a group represented by -T⁴-T⁵, wherein T⁴ represents carbonyl or sulfonyl, and T⁵ represents C₁₋₆ alkyl, C₃₋₁₀ cycloalkyl, azetidinyl, pyrrolidinyl, piperidinyl, hydroxyl, C₁₋₆ alkoxy, amino, mono-C₁₋₆ alkylamino or di-C₁₋₆ alkylamino,

where each group included in Substituent Group D may be substituted with hydroxyl, C₁₋₆ alkyl, di-C₁₋₆ alkylamino, azetidinyl or pyrrolidinyl.

[26] A method of [22], wherein R¹ represent azetidin-1-yl optionally substituted with a substituent selected from Substituent Group E, pyrrolidin-1-yl optionally substituted with a substituent selected from Substituent Group E, piperidin-1-yl optionally substituted with a substituent selected from Substituent Group E, piperazin-1-yl optionally substituted with a substituent selected from Substituent Group E, diazepan-1-yl optionally substituted with a substituent selected from Substituent Group E or morpholin-4-yl optionally substituted with a substituent selected from Substituent Group E,

wherein Substituent Group E consists of methyl, ethyl, dimethylamino, azetidinyl, pyrrolidinyl, piperidinyl and piperazinyl,

where each group included in Substituent Group E may be substituted with hydroxyl, methyl, dimethylamino, azetidinyl, pyrrolidinyl or piperidinyl.

[27] A method of [22], wherein R¹ represents azetidin-1-yl optionally substituted with a substituent selected from Substituent Group G, pyrrolidin-1-yl optionally substituted with a substituent selected from Substituent Group G, piperidin-1-yl optionally substituted with a substituent selected from Substituent Group G or piperazin-1-yl optionally substituted with a substituent selected from Substituent Group G,

wherein Substituent Group G consists of dimethylamino, azetidinyl, pyrrolidinyl, piperidinyl, piperazinyl, dimethylaminomethyl, dimethylaminoethyl, azetidin-1-ylmethyl, pyrrolidin-1-ylmethyl and piperidin-1-ylmethyl,

where each group included in Substituent Group G may be substituted with methyl or dimethylamino.

[27-1] A method of [22], wherein R¹ represents azetidin-1-yl optionally substituted with a substituent selected from Substituent Group G-1, pyrrolidin-1-yl optionally substituted with a substituent selected from Substituent Group G-1, piperidin-1-yl optionally substituted with a substituent selected from Substituent Group G-1 or piperazin-1-yl optionally substituted with a substituent selected from Substituent Group G-1,

wherein Substituent Group G-1 consists of azetidinyl, pyrrolidinyl, piperidinyl, piperazinyl, dimethylaminomethyl, dimethylaminoethyl, azetidin-1-ylmethyl, pyrrolidin-1-ylmethyl and piperidin-1-ylmethyl,

where each group included in Substituent Group G-1 may be substituted with methyl or dimethylamino.

[27-2] A method of [22], wherein R¹ represents azetidin-1-yl having dimethylamino, pyrrolidin-1-yl having dimethylamino or piperidin-1-yl having dimethylamino.
[27-3] A method of [22], wherein R¹ represents azetidin-1-yl optionally substituted with a substituent selected from Substituent Group G-2, pyrrolidin-1-yl substituted with a substituent selected from Substituent Group G-2 or piperidin-1-yl substituted with a substituent selected from Substituent Group G-2,

wherein Substituent Group G-2 consists of hydroxyl, methoxy, hydroxymethyl and dimethylaminoacetoxy.

[27-4] A method of [22], wherein R¹ represents [2-(dimethylamino)ethyl]piperazin-1-yl, 4-pyrrolidin-1-ylpiperidin-1-yl, 4-[(dimethylamino)methyl]piperidin-1-yl, 4-azetidin-1-ylpiperidin-1-yl, 4-[3-(dimethylamino)azetidin-1-yl]piperidin-1-yl, 4-(4-methylpiperazin-1-yl)piperidin-1-yl, 4-(1-methylpiperidin-4-yl)piperazin-1-yl, 4-(1-methylazetidin-3-yl)piperazin-1-yl, 4-(dimethylamino)piperidin-1-yl, 4-(azetidin-1-ylmethyl)piperidin-1-yl, 4-(pyrrolidin-1-ylmethyl)piperidin-1-yl, (3S)-3-(dimethylamino)pyrrolidin-1-yl, (3R)-3-(dimethylamino)pyrrolidin-1-yl, azetidin-1-yl, pyrrolidin-1-yl, morpholin-4-yl, 4-methylpiperazin-1-yl, 3-hydroxyazetidin-1-yl, 1,3′-biazetidin-1′-yl, 3-(hydroxymethyl)azetidin-1-yl, 3-(dimethylamino)azetidin-1-yl, 3-[(dimethylamino)methyl]azetidin-1-yl, 4-hydroxypiperidin-1-yl, 4-(hydroxymethyl)piperidin-1-yl, (3R)-3-hydroxypyrrolidin-1-yl, (3S)-3-hydroxypyrrolidin-1-yl, 3-(azetidin-1-ylmethyl)azetidin-1-yl or 3-(2-dimethylaminoacetoxy)azetidin-1-yl.
[28] A method of [22], wherein R¹ represents a group represented by the formula —NR^{11a}R^{11b}, wherein R^{11a} and R^{11b} represent the same meaning as recited in [22].
[29] A method of [22], wherein R¹ represents a group represented by the formula —NR^{11c}R^{11d}, wherein R^{11c} represents hydrogen or C₁₋₆ alkyl, and R^{11d} represents C₁₋₆ alkyl or a group represented by the formula (IV):

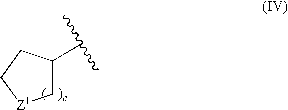

wherein c represents an integer of 1 to 3, and Z¹ represents oxygen, sulfur, carbonyl, sulfonyl or a group represented by the formula —NR^{Z1}—, wherein R^{Z1} represents hydrogen or C₁₋₆ alkyl, and R^{11d} may be substituted with a substituent selected from Substituent Group A or Substituent Group B recited in [22].
[30] A method of [22], wherein R¹ represents a group represented by the formula —NR^{11e}R^{11f}, wherein R^{11e} represents hydrogen or C₁₋₆ alkyl, and R^{11f} represents C₁₋₆ alkyl, pyrrolidin-3-yl, piperidin-3-yl, piperidin-4-yl or tetrahydropyran-4-yl, and R^{11f} may be substituted with a substituent selected from Substituent Group D recited in [25].
[31] A method of [22], wherein R¹ represents a group represented by the formula —NR^{11g}R^{11h}, wherein R^{11g} represents hydrogen or methyl, and R^{11h} represents n-propyl, n-butyl, pyrrolidin-3-yl, piperidin-3-yl, piperidin-4-yl or tetrahydropyran-4-yl, and R^{11h} may be substituted with a substituent selected from Substituent Group F,

wherein Substituent Group F consists of methyl, ethyl, n-propyl, acetyl, dimethylamino, diethylamino, azetidinyl, pyrrolidinyl and piperazinyl,

where each group included in Substituent Group F may be substituted with methyl or dimethylamino.

[32] A method of [22], wherein R¹ represents a group represented by the formula —N(CH₃)R¹¹ⁱ, wherein R¹¹ⁱ represents n-propyl, n-butyl, pyrrolidin-3-yl or piperidin-4-yl, and R¹¹ⁱ may be substituted with a substituent selected from Substituent Group H,

wherein Substituent Group H consists of dimethylamino, diethylamino, dimethylaminoethyl, dimethylaminopropyl and 1-methylazetidin-3-yl.

[33] A method of [22], wherein R¹ represents a group represented by the formula —N(CH₃)R^{11j}, wherein R^{11j} represents 1-methylpiperidin-4-yl or 1-ethylpiperidin-4-yl.
[33-1] A method of [22], wherein R¹ represents a group represented by the formula —N(CH₃)R^{11k}, wherein R^{11k} represents 3-(dimethylamino)propyl or 1-[2-(dimethylamino)ethyl]piperidin-4-yl.
[33-2] A method of [22], wherein R¹ represents methyl(1-methylpiperidin-4-yl)amino, (1-ethylpiperidin-4-yl)(methyl)amino, [3-(dimethylamino)propyl](methyl)amino or {1-[2-(dimethylamino)ethyl]piperidin-4-yl}(methyl)amino.
[34] A method of any one of [22] to [33-2], wherein R⁴, R⁵, R⁶ and R⁷ may be the same or different and each represents hydrogen, halogen or C₁₋₆ alkyl.
[35] A method of any one of [22] to [34], wherein R⁸ represents hydrogen.
[36] A method of any one of [22] to [35], wherein X represents a group represented by the formula —C(R^{10a})═, wherein R^{10a} represents hydrogen, halogen or cyano.
[37] A method of any one of [22] to [35], wherein X represents nitrogen.
[38] A method of any one of [22] to [37], wherein n represents 1.
[39] A method of any one of [22] to [38], wherein R⁹ represents mono-C₁₋₆ alkylamino optionally substituted with a substituent selected from Substituent Group A or Substituent Group B recited in [22], mono-C₃₋₁₀ cycloalkylamino optionally substituted with a substituent selected from Substituent Group A or Substituent Group B recited in [22], mono-C₆₋₁₀ arylamino optionally substituted with a substituent selected from Substituent Group A or Substituent Group B recited in [22], mono-5- to 10-membered heteroarylamino optionally substituted with a substituent selected from Substituent Group A or Substituent Group B recited in [22] or mono-4- to 10-membered non-aromatic heterocyclic amino optionally substituted with a substituent selected from Substituent Group A or Substituent Group B recited in [22].
[40] A method of any one of [22] to [38], wherein R⁹ represents mono-C₃₋₁₀ cycloalkylamino optionally substituted with a substituent selected from Substituent Group A or Substituent Group B recited in [22] or mono-C₆₋₁₀ arylamino optionally substituted with a substituent selected from Substituent Group A or Substituent Group B recited in [22].
[40-1] A method of any one of [22] to [38], wherein R⁹ represents mono-C₃₋₁₀ cycloalkylamino optionally substituted with a substituent selected from Substituent Group I or mono-C₆₋₁₀ arylamino optionally substituted with a substituent selected from Substituent Group I,

wherein Substituent Group I consists of halogen, trifluoromethyl, cyano, C₁₋₆ alkyl and C₁₋₆ alkoxy.

[40-2] A method of any one of [22] to [38], wherein R⁹ represents cyclopentylamino optionally substituted with a substituent selected from Substituent Group I recited in [40-1], cyclohexylamino optionally substituted with a substituent selected from Substituent Group I recited in [40-1], cycloheptylamino optionally substituted with a substituent selected from Substituent Group I recited in [40-1] or phenylamino optionally substituted with a substituent selected from Substituent Group I recited in [40-1].
[41] A method of [22], wherein a compound represented by the formula (I) is
(1) N-[4-({2-[({4-[2-(Dimethylamino)ethyl]piperazin-1-yl}carbonyl)amino]pyridin-4-yl}oxy)-2-fluorophenyl]-N′-(4-fluorophenyl)cyclopropane-1,1-dicarboxamide,
(2) N-(2-Fluoro-4-{[2-({[methyl(1-methylpiperidin-4-yl)amino]carbonyl}amino)pyridin-4-yl]oxy}phenyl)-N′-(4-fluorophenyl)cyclopropane-1,1-dicarboxamide,
(3) N-(4-Fluorophenyl)-N′-{2-fluoro-4-[(2-{[(4-pyrrolidin-1-ylpiperidin-1-yl)carbonyl]amino}pyridin-4-yl)oxy]phenyl}cyclopropane-1,1-dicarboxamide,
(4) N-[4-({2-[({4-[(Dimethylamino)methyl]piperidin-1-yl}carbonyl)amino]pyridin-4-yl}oxy)-2-fluorophenyl]-N′-(4-fluorophenyl)cyclopropane-1,1-dicarboxamide,
(5) N-{4-[(2-{[(4-Azetidin-1-ylpiperidin-1-yl)carbonyl]amino}pyridin-4-yl)oxy]-2-fluorophenyl}-N′-(4-fluorophenyl)cyclopropane-1,1-dicarboxamide,
(6) N-[4-({2-[({4-[3-(Dimethylamino)azetidin-1-yl]piperidin-1-yl}carbonyl)amino]pyridin-4-yl}oxy)-2-fluorophenyl]-N′-(4-fluorophenyl)cyclopropane-1,1-dicarboxamide,
(7) N-(2-Fluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide,
(8) N-(2-Fluoro-4-{[2-({[4-(1-methylpiperidin-4-yl)piperazin-1-yl]carbonyl}amino)pyridin-4-yl]oxy}phenyl)-N′-(4-fluorophenyl)cyclopropane-1,1-dicarboxamide,
(9) N-(2-Fluoro-4-{[2-({[4-(1-methylazetidin-3-yl)piperazin-1-yl]carbonyl}amino)pyridin-4-yl]oxy}phenyl)-N′-(4-fluorophenyl)cyclopropane-1,1-dicarboxamide,
(10) N-(4-{[2-({[4-(Dimethylamino)piperidin-1-yl]carbonyl}amino)pyridin-4-yl]oxy}-2-fluorophenyl)-N′-(4-fluorophenyl)cyclopropane-1,1-dicarboxamide,
(11) N-(4-{[2-({[4-(Azetidin-1-ylmethyl)piperidin-1-yl]carbonyl}amino)pyridin-4-yl]oxy}-2-fluorophenyl)-N′-(4-fluorophenyl)cyclopropane-1,1-dicarboxamide,
(12) N-(4-Fluorophenyl)-N′-(2-fluoro-4-{[2-({[4-(pyrrolidin-1-ylmethyl)piperidin-1-yl]carbonyl}amino)pyridin-4-yl]oxy}phenyl)cyclopropane-1,1-dicarboxamide,
(13) N-(4-{[2-({[(3S)-3-(Dimethylamino)pyrrolidin-1-yl]carbonyl}amino)pyridin-4-yl]oxy}-2-fluorophenyl)-N′-(4-fluorophenyl)cyclopropane-1,1-dicarboxamide,
(14) N-(4-{[2-({[(3R)-3-(Dimethylamino)pyrrolidin-1-yl]carbonyl}amino)pyridin-4-yl]oxy}-2-fluorophenyl)-N′-(4-fluorophenyl)cyclopropane-1,1-dicarboxamide,
(15) N-(2-Fluoro-4-{[2-({[methyl(1-methylpiperidin-4-yl)amino]carbonyl}amino)pyridin-4-yl]oxy}phenyl)-N′-phenylcyclopropane-1,1-dicarboxamide,
(16) N-(2-Fluoro-4-{[2-({[4-(4-methylpiperazin-1-yl)piperidin-1-yl]carbonyl}amino)pyridin-4-yl]oxy}phenyl)-N′-phenylcyclopropane-1,1-dicarboxamide,
(17) N-[4-({2-[({4-[3-(Dimethylamino)azetidin-1-yl]piperidin-1-yl}carbonyl)amino]pyridin-4-yl}oxy)-2-fluorophenyl]-N′-phenylcyclopropane-1,1-dicarboxamide,
(18) N-(4-{[2-({[(1-Ethylpiperidin-4-yl)(methyl)amino]carbonyl}amino)pyridin-4-yl]oxy}-2-fluorophenyl)-N′-phenylcyclopropane-1,1-dicarboxamide,
(19) N-[4-({2-[(Azetidin-1-ylcarbonyl)amino]pyridin-4-yl}oxy)-2-fluorophenyl]-N′-(4-fluorophenyl)cyclopropane-1,1-dicarboxamide,
(20) N-(4-Fluorophenyl)-N′-[2-fluoro-4-({2-[(pyrrolidin-1-ylcarbonyl)amino]pyridin-4-yl}oxy)phenyl]cyclopropane-1,1-dicarboxamide,
(21) N-{2-Fluoro-4-[(2-{[(3-hydroxyazetidin-1-yl)carbonyl]amino}pyridin-4-yl)oxy]phenyl}-N′-(4-fluorophenyl)cyclopropane-1,1-dicarboxamide,
(22) N-[4-({2-[(1,3′-Biazetidin-1′-ylcarbonyl)amino]pyridin-4-yl}oxy)-2-fluorophenyl]-N′-(4-fluorophenyl)cyclopropane-1,1-dicarboxamide,
(23) N-(2-Fluoro-4-{[2-({[3-(hydroxymethyl)azetidin-1-yl]carbonyl}amino)pyridin-4-yl]oxy}phenyl)-N′-(4-fluorophenyl)cyclopropane-1,1-dicarboxamide,
(24) N-(4-{[2-({[3-(Dimethylamino)azetidin-1-yl]carbonyl}amino)pyridin-4-yl]oxy}-2-fluorophenyl)-N′-(4-fluorophenyl)cyclopropane-1,1-dicarboxamide,
(25) N-[4-({2-[({3-[(Dimethylamino)methyl] azetidin-1-yl}carbonyl)amino]pyridin-4-yl}oxy)-2-fluorophenyl]-N′-(4-fluorophenyl)cyclopropane-1,1-dicarboxamide,
(26) N-{2-Fluoro-4-[(2-{[(4-hydroxypiperidin-1-yl)carbonyl]amino}pyridin-4-yl)oxy]phenyl}-N′-(4-fluorophenyl)cyclopropane-1,1-dicarboxamide,
(27) N-(2-Fluoro-4-{[2-({[4-(hydroxymethyl)piperidin-1-yl]carbonyl}amino)pyridin-4-yl]oxy}phenyl)-N′-(4-fluorophenyl)cyclopropane-1,1-dicarboxamide,
(28) N-(2-Fluoro-4-{[2-({[(3R)-3-hydroxypyrrolidin-1-yl]carbonyl}amino)pyridin-4-yl]oxy}phenyl)-N′-(4-fluorophenyl)cyclopropane-1,1-dicarboxamide,
(29) N-(2-Fluoro-4-{[2-({[(3S)-3-hydroxypyrrolidin-1-yl]carbonyl}amino)pyridin-4-yl]oxy}phenyl)-N′-(4-fluorophenyl)cyclopropane-1,1-dicarboxamide,
(30) N-[4-({2-[(Azetidin-1-ylcarbonyl)amino]pyridin-4-yl}oxy)-2,5-difluorophenyl]-N′-(4-fluorophenyl)cyclopropane-1,1-dicarboxamide,
(31) N-{2,5-Difluoro-4-[(2-{[(3-hydroxyazetidin-1-yl)carbonyl]amino}pyridin-4-yl)oxy]phenyl}-N′-(4-fluorophenyl)cyclopropane-1,1-dicarboxamide,
(32) N-(2,5-Difluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide,
(33) N-[2,5-Difluoro-4-({2-[({3-[(dimethylamino)methyl]azetidin-1-yl}carbonyl)amino]pyridin-4-yl}oxy)phenyl]-N′-(4-fluorophenyl)cyclopropane-1,1-dicarboxamide,
(34) N-(2,5-Difluoro-4-{[2-({[methyl(1-methylpiperidin-4-yl)amino]carbonyl}amino)pyridin-4-yl]oxy}phenyl)-N′-(4-fluorophenyl)cyclopropane-1,1-dicarboxamide,
(35) N-{4-[(2-{[3-(Azetidin-1-ylmethyl)azetidin-1-ylcarbonyl]amino}pyridin-4-yl)oxy]-2,5-difluorophenyl}-N′-(4-fluorophenyl)cyclopropane-1,1-dicarboxamide,
(36) N-(2,5-Difluoro-4-{[2-({[3-(hydroxymethyl)azetidin-1-yl]carbonyl}amino)pyridin-4-yl]oxy}phenyl)-N′-(4-fluorophenyl)cyclopropane-1,1-dicarboxamide,
(37) N-{2,5-Difluoro-4-[(4-{[(3-hydroxyazetidin-1-yl)carbonyl]amino}pyrimidin-6-yl)oxy]phenyl}-N′-(4-fluorophenyl)cyclopropane-1,1-dicarboxamide,
(38) N-[4-({4-[({3-[(Dimethylamino)methyl] azetidin-1-yl}carbonyl)amino]pyrimidin-6-yl}oxy)-2,5-difluorophenyl]-N′-(4-fluorophenyl)cyclopropane-1,1-dicarboxamide,
(39) N-(2,5-Difluoro-4-{[4-({[3-(hydroxymethyl)azetidin-1-yl]carbonyl}amino)pyrimidin-6-yl]oxy}phenyl)-N′-(4-fluorophenyl)cyclopropane-1,1-dicarboxamide,
(40) N-(2,5-Difluoro-4-{[4-({[methyl(1-methylpiperidin-4-yl)amino]carbonyl}amino)pyrimidin-6-yl]oxy}phenyl)-N′-(4-fluorophenyl)cyclopropane-1,1-dicarboxamide,
(41) N-(2,5-Difluoro-4-{[4-({[4-(4-methylpiperazin-1-yl)piperidin-1-yl]carbonyl}amino)pyrimidin-6-yl]oxy}phenyl)-N′-(4-fluorophenyl)cyclopropane-1,1-dicarboxamide,
(42) N-(4-{[2-({[4-(Dimethylamino)piperidin-1-yl]carbonyl}amino)pyridin-4-yl]oxy}-2,5-difluorophenyl)-N′-(4-fluorophenyl)cyclopropane-1,1-dicarboxamide,
(43) N-{2,5-Difluoro-4-[(2-{[(4-methylpiperazin-1-yl)carbonyl]amino}pyridin-4-yl]oxy}phenyl)-N′-(4-fluorophenyl)cyclopropane-1,1-dicarboxamide,
(44) N-{2,5-Difluoro-4-[(2-{[(4-hydroxypiperidin-1-yl)carbonyl]amino}pyridin-4-yl)oxy]phenyl}-N′-(4-fluorophenyl)cyclopropane-1,1-dicarboxamide,
(45) N-{4-[(2-{[(4-Azetidin-1-ylpiperidin-1-yl)carbonyl]amino}pyridin-4-yl)oxy]oxy}-2,5-difluorophenyl}-N′-(4-fluorophenyl)cyclopropane-1,1-dicarboxamide,
(46) N-(2,5-Difluoro-4-{[2-({[3-(2-dimethylaminoacetoxy)azetidin-1-yl]carbonyl}amino)pyridin-4-yl]oxy}phenyl)-N′-(4-fluorophenyl)cyclopropane-1,1-dicarboxamide,
(47) N-(2,5-Difluoro-4-{[2-({[(3S)-3-hydroxypyrrolidin-1-yl]carbonyl}amino)pyridin-4-yl]oxy}phenyl)-N′-(4-fluorophenyl)cyclopropane-1,1-dicarboxamide or
(48) N-(2,5-Difluoro-4-{[2-({[(3R)-3-hydroxypyrrolidin-1-yl]carbonyl}amino)pyridin-4-yl]oxy}phenyl)-N′-(4-fluorophenyl)cyclopropane-1,1-dicarboxamide.
   [42] A method of [22], wherein a compound represented by the formula (I) is
(1) N-(2-Fluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide
(2) N-[4-({2-[(Azetidin-1-ylcarbonyl)amino]pyridin-4-yl}oxy)-2-fluorophenyl]-N′-(4-fluorophenyl)cyclopropane-1,1-dicarboxamide
(3) N-{2,5-Difluoro-4-[(2-{[(3-hydroxyazetidin-1-yl)carbonyl]amino}pyridin-4-yl)oxy]phenyl}-N′-(4-fluorophenyl)cyclopropane-1,1-dicarboxamide
(4) N-(2,5-Difluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide
(5) N-(2,5-Difluoro-4-{[2-({[methyl(1-methylpiperidin-4-yl)amino]carbonyl}amino)pyridin-4-yl]oxy}phenyl)-N′-(4-fluorophenyl)cyclopropane-1,1-dicarboxamide or
(6) N-(2,5-Difluoro-4-{[2-({[3-(hydroxymethyl)azetidin-1-yl]carbonyl}amino)pyridin-4-yl]oxy}phenyl)-N′-(4-fluorophenyl)cyclopropane-1,1-dicarboxamide.
   [43] A compound represented by the following formula, a salt thereof or a hydrate of the foregoing for treating a disease associated with activation of VEGFR-1, VEGFR-2, VEGFR-3, RON, RET and/or KIT:

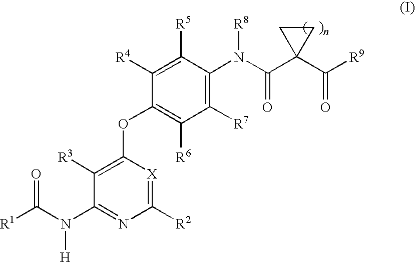

wherein R¹ represents a 3- to 10-membered non-aromatic heterocyclic group wherein the group is limited to a group having nitrogen as a ring constituent atom and the nitrogen having a bonding hand, or a group represented by the formula —NR^{11a}R^{11b}, wherein R^{11a} and R^{11b} may be the same or different and each represents hydrogen, C₁₋₆ alkyl, C₃₋₆ alkenyl, C₃₋₆ alkynyl, C₃₋₁₀ cycloalkyl, C₆₋₁₀ aryl, 5- to 10-membered heteroaryl or a 4- to 10-membered non-aromatic heterocyclic group, and R^{11a} and R^{11b} may be substituted with a substituent selected from Substituent Group A or Substituent Group B and R¹ may be substituted with a substituent selected from Substituent Group A or Substituent Group B;

R² and R³ represent hydrogen;

R⁴, R⁵, R⁶ and R⁷ may be the same or different and each represents hydrogen, halogen, hydroxyl, cyano, trifluoromethyl, C₁₋₆ alkyl, C₂₋₆ alkenyl, C₂₋₆ alkynyl, C₁₋₆ alkoxy, amino, mono-C₁₋₆ alkylamino, di-C₁₋₆ alkylamino or a group represented by the formula —CO—R¹², wherein R¹² represents hydrogen, hydroxyl, C₁₋₆ alkyl, C₁₋₆ alkoxy, amino, mono-C₁₋₆ alkylamino or di-C₁₋₆ alkylamino;

R⁸ represents hydrogen or C₁₋₆ alkyl;

R⁹ represents a 3- to 10-membered non-aromatic heterocyclic group wherein the group is limited to a group having nitrogen as a ring constituent atom and the nitrogen having a bonding hand, or a group represented by the formula —NR^{11a}R^{11b}, wherein R^{11a} and R^{11b} represent the same meaning as described above and R⁹ may be substituted with a substituent selected from Substituent Group A or Substituent Group B;

n represents an integer of 1 or 2; and

X represents a group represented by the formula —C(R¹⁰)= or nitrogen, wherein R¹⁰ represents hydrogen, halogen, cyano, C₁₋₆ alkyl, C₂₋₆ alkenyl, C₂₋₆ alkynyl or a group represented by the formula —CO—R¹² wherein R¹² represents the same meaning as recited above;

wherein Substituent Group A consists of halogen, hydroxyl, mercapto, nitro, cyano and oxo;

wherein Substituent Group B consists of C₁₋₆ alkyl, C₂₋₆ alkenyl, C₂₋₆ alkynyl, C₃₋₁₀ cycloalkyl, C₆₋₁₀ aryl, 5- to 10-membered heteroaryl, a 3- to 10-membered non-aromatic heterocyclic group, C₁₋₆ alkoxy, C₃₋₆ alkenyloxy, C₃₋₆ alkynyloxy, C₃₋₁₀ cycloalkoxy, C₆₋₁₀ aryloxy, 5- to 10-membered heteroaryloxy, 4- to 10-membered non-aromatic heterocyclicoxy, C₁₋₆ alkylthio, C₃₋₆ alkenylthio, C₃₋₆ alkynylthio, C₃₋₁₀ Cycloalkylthio, C₆₋₁₀ arylthio, 5- to 10-membered heteroarylthio, 4- to 10-membered non-aromatic heterocyclicthio and a group represented by the formula -T¹-T²-T³, and each group in Substituent Group B may be substituted with a substituent selected from Substituent Group C, wherein T¹ represents a direct bond or C₁₋₆ alkylene, T² represents carbonyl, sulfinyl, sulfonyl, a group represented by the formula —C(═O)—O—, a group represented by the formula —O—C(═O)—, a group represented by the formula —SO₂—O—, a group represented by the formula —O—SO₂—, a group represented by the formula —NR^{T1}—, a group represented by the formula —C(═O)—NR^{T1}—, a group represented by the formula —NR^{T1}—C(═O)—, a group represented by the formula —SO₂—NR^{T1}— or a group represented by the formula —NR^{T1}—SO₂—, T³ represents hydrogen, C₁₋₆ alkyl, C₃₋₆ alkenyl, C₃₋₆ alkynyl, C₃₋₁₀ cycloalkyl, C₆- to aryl, 5- to 10-membered heteroaryl or a 4- to 10-membered non-aromatic heterocyclic group, and R^{T1} represents hydrogen or C₁₋₆ alkyl; and

wherein Substituent Group C consists of halogen, hydroxyl, mercapto, nitro, cyano, oxo, C₁₋₆ alkyl, C₂₋₆ alkenyl, C₂₋₆ alkynyl, C₃₋₁₀ cycloalkyl, C₆₋₁₀ aryl, 5- to 10-membered heteroaryl, a 3- to 10-membered non-aromatic heterocyclic group, C₁₋₆ alkoxy, C₁₋₆ alkylthio, mono-C₁₋₆ alkylamino and di-C₁₋₆ alkylamino.

[44] A compound represented by the following formula, a salt thereof or a hydrate of the foregoing of [43], wherein R¹ represents a 3- to 10-membered non-aromatic heterocyclic group optionally substituted with a substituent selected from Substituent Group A or Substituent Group B recited in [43], wherein the group is limited to a group having nitrogen as a ring constituent atom and the nitrogen having a bonding hand.
[45] A compound represented by the following formula, a salt thereof or a hydrate of the foregoing of [43], wherein R¹ represents a group represented by the formula (II):

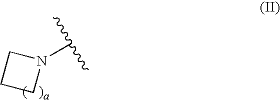

wherein a represents an integer of 1 to 4;
or a group represented by the formula (III):

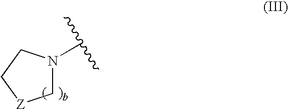

wherein b represents an integer of 1 to 3, and Z represents oxygen, sulfur, carbonyl, sulfonyl, or a group represented by the formula —NR^{Z}—, wherein R^{Z} represents hydrogen or C₁₋₆ alkyl, and the groups represented by the formula (II) or (III) may be substituted with a substituent selected from Substituent Group A or Substituent Group B recited in [43].
[46] A compound represented by the following formula, a salt thereof or a hydrate of the foregoing of [43], wherein R¹ represents azetidin-1-yl optionally substituted with a substituent selected from Substituent Group D, pyrrolidin-1-yl optionally substituted with a substituent selected from Substituent Group D, piperidin-1-yl optionally substituted with a substituent selected from Substituent Group D, azepan-1-yl optionally substituted with a substituent selected from Substituent Group D, piperazin-1-yl optionally substituted with a substituent selected from Substituent Group D, diazepan-1-yl optionally substituted with a substituent selected from Substituent Group D, morpholin-4-yl optionally substituted with a substituent selected from Substituent Group D, thiomorpholin-4-yl optionally substituted with a substituent selected from Substituent Group D, 1,1-dioxothiomorpholin-4-yl optionally substituted with a substituent selected from Substituent Group D,

wherein Substituent Group D consists of halogen, hydroxyl, mercapto, cyano, formyl, oxo, C₁₋₆ alkyl, C₃₋₁₀ cycloalkyl, C₁₋₆ alkoxy, amino, mono-C₁₋₆ alkylamino, di-C₁₋₆ alkylamino, azetidinyl, pyrrolidinyl, piperidinyl, piperazinyl, diazepanyl and a group represented by -T⁴-T⁵, wherein T⁴ represents carbonyl or sulfonyl, and T⁵ represents C₁₋₆ alkyl, C₃- to cycloalkyl, azetidinyl, pyrrolidinyl, piperidinyl, hydroxyl, C₁₋₆ alkoxy, amino, mono-C₁₋₆ alkylamino or di-C₁₋₆ alkylamino, where each group included in Substituent Group D may be substituted with hydroxyl, C₁₋₆ alkyl, di-C₁₋₆ alkylamino, azetidinyl or pyrrolidinyl.

[47] A compound represented by the following formula, a salt thereof or a hydrate of the foregoing of [43], wherein R¹ represent azetidin-1-yl optionally substituted with a substituent selected from Substituent Group E, pyrrolidin-1-yl optionally substituted with a substituent selected from Substituent Group E, piperidin-1-yl optionally substituted with a substituent selected from Substituent Group E, piperazin-1-yl optionally substituted with a substituent selected from Substituent Group E, diazepan-1-yl optionally substituted with a substituent selected from Substituent Group E or morpholin-4-yl optionally substituted with a substituent selected from Substituent Group E,

wherein Substituent Group E consists of methyl, ethyl, dimethylamino, azetidinyl, pyrrolidinyl, piperidinyl and piperazinyl,

where each group included in Substituent Group E may be substituted with hydroxyl, methyl, dimethylamino, azetidinyl, pyrrolidinyl or piperidinyl.

[48] A compound represented by the following formula, a salt thereof or a hydrate of the foregoing of [43], wherein R¹ represents azetidin-1-yl optionally substituted with a substituent selected from Substituent Group G, pyrrolidin-1-yl optionally substituted with a substituent selected from Substituent Group G, piperidin-1-yl optionally substituted with a substituent selected from Substituent Group G or piperazin-1-yl optionally substituted with a substituent selected from Substituent Group G,

wherein Substituent Group G consists of dimethylamino, azetidinyl, pyrrolidinyl, piperidinyl, piperazinyl, dimethylaminomethyl, dimethylaminoethyl, azetidin-1-ylmethyl, pyrrolidin-1-ylmethyl and piperidin-1-ylmethyl,

where each group included in Substituent Group G may be substituted with methyl or dimethylamino.

[48-1] A compound represented by the following formula, a salt thereof or a hydrate of the foregoing of [43], wherein R¹ represents azetidin-1-yl optionally substituted with a substituent selected from Substituent Group G-1, pyrrolidin-1-yl optionally substituted with a substituent selected from Substituent Group G-1, piperidin-1-yl optionally substituted with a substituent selected from Substituent Group G-1 or piperazin-1-yl optionally substituted with a substituent selected from Substituent Group G-1,

wherein Substituent Group G-1 consists of azetidinyl, pyrrolidinyl, piperidinyl, piperazinyl, dimethylaminomethyl, dimethylaminoethyl, azetidin-1-ylmethyl, pyrrolidin-1-ylmethyl and piperidin-1-ylmethyl,

where each group included in Substituent Group G-1 may be substituted with methyl or dimethylamino.

[48-2] A compound represented by the following formula, a salt thereof or a hydrate of the foregoing of [43], wherein R¹ represents azetidin-1-yl having dimethylamino, pyrrolidin-1-yl having dimethylamino or piperidin-1-yl having dimethylamino.
[48-3] A compound represented by the following formula, a salt thereof or a hydrate of the foregoing of [43], wherein R¹ represents azetidin-1-yl optionally substituted with a substituent selected from Substituent Group G-2, pyrrolidin-1-yl substituted with a substituent selected from Substituent Group G-2 or piperidin-1-yl substituted with a substituent selected from Substituent Group G-2,

wherein Substituent Group G-2 consists of hydroxyl, methoxy, hydroxymethyl and dimethylaminoacetoxy.

[48-4] A compound represented by the following formula, a salt thereof or a hydrate of the foregoing of [43], wherein R¹ represents [2-(dimethylamino)ethyl]piperazin-1-yl, 4-pyrrolidin-1-ylpiperidin-1-yl, 4-[(dimethylamino)methyl]piperidin-1-yl, 4-azetidin-1-ylpiperidin-1-yl, 4-[3-(dimethylamino)azetidin-1-yl]piperidin-1-yl, 4-(4-methylpiperazin-1-yl)piperidin-1-yl, 4-(1-methylpiperidin-4-yl)piperazin-1-yl, 4-(1-methylazetidin-3-yl)piperazin-1-yl, 4-(dimethylamino)piperidin-1-yl, 4-(azetidin-1-ylmethyl)piperidin-1-yl, 4-(pyrrolidin-1-ylmethyl)piperidin-1-yl, (3S)-3-(dimethylamino)pyrrolidin-1-yl, (3R)-3-(dimethylamino)pyrrolidin-1-yl, azetidin-1-yl, pyrrolidin-1-yl, morpholin-4-yl, 4-methylpiperazin-1-yl, 3-hydroxyazetidin-1-yl, 1,3′-biazetidin-1′-yl, 3-(hydroxymethyl)azetidin-1-yl, 3-(dimethylamino)azetidin-1-yl, 3-[(dimethylamino)methyl]azetidin-1-yl, 4-hydroxypiperidin-1-yl, 4-(hydroxymethyl)piperidin-1-yl, (3R)-3-hydroxypyrrolidin-1-yl, (3S)-3-hydroxypyrrolidin-1-yl, 3-(azetidin-1-ylmethyl)azetidin-1-yl or 3-(2-dimethylaminoacetoxy)azetidin-1-yl.
[49] A compound represented by the following formula, a salt thereof or a hydrate of the foregoing of [43], wherein R¹ represents a group represented by the formula —NR^{11a}R^{11b}, wherein R^{11a} and R^{11b} represent the same meaning as recited in [43].
[50] A compound represented by the following formula, a salt thereof or a hydrate of the foregoing of [43], wherein R¹ represents a group represented by the formula —NR^{11c}R^{11d}, wherein R^{11c} represents hydrogen or C₁₋₆ alkyl, and R^{11d} represents C₁₋₆ alkyl or a group represented by the formula (IV):

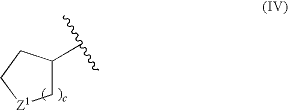

wherein c represents an integer of 1 to 3, and Z¹ represents oxygen, sulfur, carbonyl, sulfonyl or a group represented by the formula —NR^{Z1}—, wherein R^{Z1} represents hydrogen or C₁₋₆ alkyl, and R^{11d} may be substituted with a substituent selected from Substituent Group A or Substituent Group B recited in [43].
[51] A compound represented by the following formula, a salt thereof or a hydrate of the foregoing of [43], wherein R¹ represents a group represented by the formula —NR^{11e}R^{11f}, wherein R^{11e} represents hydrogen or C₁₋₆ alkyl, and R^{11f} represents C₁₋₆ alkyl, pyrrolidin-3-yl, piperidin-3-yl, piperidin-4-yl or tetrahydropyran-4-yl, and R^{11f} may be substituted with a substituent selected from Substituent Group D recited in [46].
[52] A compound represented by the following formula, a salt thereof or a hydrate of the foregoing of [43], wherein R¹ represents a group represented by the formula —NR^{11g}R^{11h}, wherein R^{11g} represents hydrogen or methyl, and R^{11h} represents n-propyl, n-butyl, pyrrolidin-3-yl, piperidin-3-yl, piperidin-4-yl or tetrahydropyran-4-yl, and R^{11h} may be substituted with a substituent selected from Substituent Group F,

wherein Substituent Group F consists of methyl, ethyl, n-propyl, acetyl, dimethylamino, diethylamino, azetidinyl, pyrrolidinyl and piperazinyl,

where each group included in Substituent Group F may be substituted with methyl or dimethylamino.

[53] A compound represented by the following formula, a salt thereof or a hydrate of the foregoing of [43], wherein R¹ represents a group represented by the formula —N(CH₃)R¹¹ⁱ, wherein R¹¹ⁱ represents n-propyl, n-butyl, pyrrolidin-3-yl or piperidin-4-yl, and R¹¹ⁱ may be substituted with a substituent selected from Substituent Group H,

wherein Substituent Group H consists of dimethylamino, diethylamino, dimethylaminoethyl, dimethylaminopropyl and 1-methylazetidin-3-yl.

[54] A compound represented by the following formula, a salt thereof or a hydrate of the foregoing of [43], wherein R¹ represents a group represented by the formula —N(CH₃)R^{11j}, wherein R^{11j} represents 1-methylpiperidin-4-yl or 1-ethylpiperidin-4-yl.
[54-1] A compound represented by the following formula, a salt thereof or a hydrate of the foregoing of [43], wherein R¹ represents a group represented by the formula —N(CH₃)R^{11k}, wherein R^{11k} represents 3-(dimethylamino)propyl or 1-[2-(dimethylamino)ethyl]piperidin-4-yl.
[54-2] A compound represented by the following formula, a salt thereof or a hydrate of the foregoing of [43], wherein R¹ represents methyl(1-methylpiperidin-4-yl)amino, (1-ethylpiperidin-4-yl)(methyl)amino, [3-(dimethylamino)propyl] (methyl)amino or {1-[2-(dimethylamino)ethyl]piperidin-4-yl}(methyl)amino.
[55] A compound represented by the following formula, a salt thereof or a hydrate of the foregoing of any one of [43] to [54-2], wherein R⁴, R⁵, R⁶ and R⁷ may be the same or different and each represents hydrogen, halogen or C₁₋₆ alkyl.
[56] A compound represented by the following formula, a salt thereof or a hydrate of the foregoing of any one of [43] to [55], wherein R⁸ represents hydrogen.
[57] A compound represented by the following formula, a salt thereof or a hydrate of the foregoing of any one of [43] to [56], wherein X represents a group represented by the formula —C(R^{10a})═, wherein R^{10a} represents hydrogen, halogen or cyano.
[58] A compound represented by the following formula, a salt thereof or a hydrate of the foregoing of any one of [43] to [56], wherein X represents nitrogen.
[59] A compound represented by the following formula, a salt thereof or a hydrate of the foregoing of any one of [43] to [58], wherein n represents 1.
[60] A compound represented by the following formula, a salt thereof or a hydrate of the foregoing of any one of [43] to [59], wherein R⁹ represents mono-C₁₋₆ alkylamino optionally substituted with a substituent selected from Substituent Group A or Substituent Group B recited in [43], mono-C₃₋₁₀ cycloalkylamino optionally substituted with a substituent selected from Substituent Group A or Substituent Group B recited in [43], mono-C₆₋₁₀ arylamino optionally substituted with a substituent selected from Substituent Group A or Substituent Group B recited in [43], mono-5- to 10-membered heteroarylamino optionally substituted with a substituent selected from Substituent Group A or Substituent Group B recited in [43] or mono-4- to 10-membered non-aromatic heterocyclic amino optionally substituted with a substituent selected from Substituent Group A or Substituent Group B recited in [43].
[61] A compound represented by the following formula, a salt thereof or a hydrate of the foregoing of any one of [43] to [59], wherein R⁹ represents mono-C₃₋₁₀ cycloalkylamino optionally substituted with a substituent selected from Substituent Group A or Substituent Group B recited in [1] or mono-C₆₋₁₀ arylamino optionally substituted with a substituent selected from Substituent Group A or Substituent Group B recited in [1].
[61-1] A compound represented by the following formula, a salt thereof or a hydrate of the foregoing of any one of [43] to [59], wherein R⁹ represents mono-C₃₋₁₀ cycloalkylamino optionally substituted with a substituent selected from Substituent Group I or mono-C₆₋₁₀ arylamino optionally substituted with a substituent selected from Substituent Group I,

wherein Substituent Group I consists of halogen, trifluoromethyl, cyano, C₁₋₆ alkyl and C₁₋₆ alkoxy.

[61-2] A compound represented by the following formula, a salt thereof or a hydrate of the foregoing of any one of [43] to [59], wherein R⁹ represents cyclopentylamino optionally substituted with a substituent selected from Substituent Group I recited in [61-1], cyclohexylamino optionally substituted with a substituent selected from Substituent Group I recited in [61-1], cycloheptylamino optionally substituted with a substituent selected from Substituent Group I recited in [61-1] or phenylamino optionally substituted with a substituent selected from Substituent Group I recited in [61-1].
[62] A compound represented by the following formula, a salt thereof or a hydrate of the foregoing of [43], wherein a compound represented by the formula (I) is
(1) N-[4-({2-[({4-[2-(Dimethylamino)ethyl]piperazin-1-yl}carbonyl)amino]pyridin-4-yl}oxy)-2-fluorophenyl]-N′-(4-fluorophenyl)cyclopropane-1,1-dicarboxamide,
(2) N-(2-Fluoro-4-{[2-({[methyl(1-methylpiperidin-4-yl)amino]carbonyl}amino)pyridin-4-yl]oxy}phenyl)-N′-(4-fluorophenyl)cyclopropane-1,1-dicarboxamide,
(3) N-(4-Fluorophenyl)-N′-{2-fluoro-4-[(2-{[(4-pyrrolidin-1-ylpiperidin-1-yl)carbonyl]amino}pyridin-4-yl)oxy]phenyl}cyclopropane-1,1-dicarboxamide,
(4) N-[4-({2-[({4-[(Dimethylamino)methyl]piperidin-1-yl}carbonyl)amino]pyridin-4-yl}oxy)-2-fluorophenyl]-N′-(4-fluorophenyl)cyclopropane-1,1-dicarboxamide,
(5) N-{4-[(2-{[(4-Azetidin-1-ylpiperidin-1-yl)carbonyl]amino}pyridin-4-yl)oxy]-2-fluorophenyl}-N′-(4-fluorophenyl)cyclopropane-1,1-dicarboxamide,
(6) N-[4-({2-[({4-[3-(Dimethylamino)azetidin-1-yl]piperidin-1-yl}carbonyl)amino]pyridin-4-yl}oxy)-2-fluorophenyl]-N′-(4-fluorophenyl)cyclopropane-1,1-dicarboxamide,
(7) N-(2-Fluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide,
(8) N-(2-Fluoro-4-{[2-({[4-(1-methylpiperidin-4-yl)piperazin-1-yl]carbonyl}amino)pyridin-4-yl]oxy}phenyl)-N′-(4-fluorophenyl)cyclopropane-1,1-dicarboxamide,
(9) N-(2-Fluoro-4-{[2-({[4-(1-methylazetidin-3-yl)piperazin-1-yl]carbonyl}amino)pyridin-4-yl]oxy}phenyl)-N′-(4-fluorophenyl)cyclopropane-1,1-dicarboxamide,
(10) N-(4-{[2-({[4-(Dimethylamino)piperidin-1-yl]carbonyl}amino)pyridin-4-yl]oxy}-2-fluorophenyl)-N′-(4-fluorophenyl)cyclopropane-1,1-dicarboxamide,
(11) N-(4-{[2-({[4-(Azetidin-1-ylmethyl)piperidin-1-yl]carbonyl}amino)pyridin-4-yl]oxy}-2-fluorophenyl)-N′-(4-fluorophenyl)cyclopropane-1,1-dicarboxamide,
(12) N-(4-Fluorophenyl)-N′-(2-fluoro-4-{[2-({[4-(pyrrolidin-1-ylmethyl)piperidin-1-yl]carbonyl}amino)pyridin-4-yl]oxy}phenyl)cyclopropane-1,1-dicarboxamide,
(13) N-(4-{[2-({[(3S)-3-(Dimethylamino)pyrrolidin-1-yl]carbonyl}amino)pyridin-4-yl]oxy}-2-fluorophenyl)-N′-(4-fluorophenyl)cyclopropane-1,1-dicarboxamide,
(14) N-(4-{[2-({[(3R)-3-(Dimethylamino)pyrrolidin-1-yl]carbonyl}amino)pyridin-4-yl]oxy}-2-fluorophenyl)-N′-(4-fluorophenyl)cyclopropane-1,1-dicarboxamide,
(15) N-(2-Fluoro-4-{[2-({[methyl(1-methylpiperidin-4-yl)amino]carbonyl}amino)pyridin-4-yl]oxy}phenyl)-N′-phenylcyclopropane-1,1-dicarboxamide,
(16) N-(2-Fluoro-4-{[2-({[4-(4-methylpiperazin-1-yl)piperidin-1-yl]carbonyl}amino)pyridin-4-yl]oxy}phenyl)-N′-phenylcyclopropane-1,1-dicarboxamide,
(17) N-[4-({2-[({4-[3-(Dimethylamino)azetidin-1-yl]piperidin-1-yl}carbonyl)amino]pyridin-4-yl}oxy)-2-fluorophenyl]-N′-phenylcyclopropane-1,1-dicarboxamide,
(18) N-(4-{[2-({[(1-Ethylpiperidin-4-yl)(methyl)amino]carbonyl}amino)pyridin-4-yl]oxy}-2-fluorophenyl)-N′-phenylcyclopropane-1,1-dicarboxamide,
(19) N-[4-({2-[(Azetidin-1-ylcarbonyl)amino]pyridin-4-yl}oxy)-2-fluorophenyl]-N′-(4-fluorophenyl)cyclopropane-1,1-dicarboxamide,
(20) N-(4-Fluorophenyl)-N′-[2-fluoro-4-({2-[(pyrrolidin-1-ylcarbonyl)amino]pyridin-4-yl}oxy)phenyl]cyclopropane-1,1-dicarboxamide,
(21) N-{2-Fluoro-4-[(2-{[(3-hydroxyazetidin-1-yl)carbonyl]amino}pyridin-4-yl)oxy]phenyl}-N′-(4-fluorophenyl)cyclopropane-1,1-dicarboxamide,
(22) N-[4-({2-[(1,3′-Biazetidin-1′-ylcarbonyl)amino]pyridin-4-yl}oxy)-2-fluorophenyl]-N′-(4-fluorophenyl)cyclopropane-1,1-dicarboxamide,
(23) N-(2-Fluoro-4-{[2-({[3-(hydroxymethyl)azetidin-1-yl]carbonyl}amino)pyridin-4-yl]oxy}phenyl)-N′-(4-fluorophenyl)cyclopropane-1,1-dicarboxamide,
(24) N-(4-{[2-({[3-(Dimethylamino)azetidin-1-yl]carbonyl}amino)pyridin-4-yl]oxy}-2-fluorophenyl)-N′-(4-fluorophenyl)cyclopropane-1,1-dicarboxamide,
(25) N-[4-({2-[({3-[(Dimethylamino)methyl] azetidin-1-yl}carbonyl)amino]pyridin-4-yl}oxy)-2-fluorophenyl]-N′-(4-fluorophenyl)cyclopropane-1,1-dicarboxamide,
(26) N-{2-Fluoro-4-[(2-{[(4-hydroxypiperidin-1-yl)carbonyl]amino}pyridin-4-yl)oxy]phenyl}-N′-(4-fluorophenyl)cyclopropane-1,1-dicarboxamide,
(27) N-(2-Fluoro-4-{[2-({[4-(hydroxymethyl)piperidin-1-yl]carbonyl}amino)pyridin-4-yl]oxy}phenyl)-N′-(4-fluorophenyl)cyclopropane-1,1-dicarboxamide,
(28) N-(2-Fluoro-4-{[2-({[(3R)-3-hydroxypyrrolidin-1-yl]carbonyl}amino)pyridin-4-yl]oxy}phenyl)-N′-(4-fluorophenyl)cyclopropane-1,1-dicarboxamide,
(29) N-(2-Fluoro-4-{[2-({[(3S)-3-hydroxypyrrolidin-1-yl]carbonyl}amino)pyridin-4-yl]oxy}phenyl)-N′-(4-fluorophenyl)cyclopropane-1,1-dicarboxamide,
(30) N-[4-({2-[(Azetidin-1-ylcarbonyl)amino]pyridin-4-yl}oxy)-2,5-difluorophenyl]-N′-(4-fluorophenyl)cyclopropane-1,1-dicarboxamide,
(31) N-{2,5-Difluoro-4-[(2-{[(3-hydroxyazetidin-1-yl)carbonyl]amino}pyridin-4-yl)oxy]phenyl}-N′-(4-fluorophenyl)cyclopropane-1,1-dicarboxamide,
(32) N-(2,5-Difluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide,
(33) N-[2,5-Difluoro-4-({2-[({3-[(dimethylamino)methyl] azetidin-1-yl}carbonyl)amino]pyridin-4-yl}oxy)phenyl]-N′-(4-fluorophenyl)cyclopropane-1,1-dicarboxamide,
(34) N-(2,5-Difluoro-4-{[2-({[methyl(1-methylpiperidin-4-yl)amino]carbonyl}amino)pyridin-4-yl]oxy}phenyl)-N′-(4-fluorophenyl)cyclopropane-1,1-dicarboxamide,
(35) N-{4-[(2-{[3-(Azetidin-1-ylmethyl)azetidin-1-ylcarbonyl]amino}pyridin-4-yl)oxy]-2,5-difluorophenyl}-N′-(4-fluorophenyl)cyclopropane-1,1-dicarboxamide,
(36) N-(2,5-Difluoro-4-{[2-({[3-(hydroxymethyl)azetidin-1-yl]carbonyl}amino)pyridin-4-yl]oxy}phenyl)-N′-(4-fluorophenyl)cyclopropane-1,1-dicarboxamide,
(37) N-{2,5-Difluoro-4-[(4-{[(3-hydroxyazetidin-1-yl)carbonyl]amino}pyrimidin-6-yl)oxy]phenyl}-N′-(4-fluorophenyl)cyclopropane-1,1-dicarboxamide,
(38) N-[4-({4-[({3-[(Dimethylamino)methyl] azetidin-1-yl}carbonyl)amino]pyrimidin-6-yl}oxy)-2,5-difluorophenyl]-N′-(4-fluorophenyl)cyclopropane-1,1-dicarboxamide,
(39) N-(2,5-Difluoro-4-{[4-({[3-(hydroxymethyl)azetidin-1-yl]carbonyl}amino)pyrimidin-6-yl]oxy}phenyl)-N′-(4-fluorophenyl)cyclopropane-1,1-dicarboxamide,
(40) N-(2,5-Difluoro-4-{[4-({[methyl(1-methylpiperidin-4-yl)amino]carbonyl}amino)pyrimidin-6-yl]oxy}phenyl)-N′-(4-fluorophenyl)cyclopropane-1,1-dicarboxamide,
(41) N-(2,5-Difluoro-4-{[4-({[4-(4-methylpiperazin-1-yl)piperidin-1-yl]carbonyl}amino)pyrimidin-6-yl]oxy}phenyl)-N′-(4-fluorophenyl)cyclopropane-1,1-dicarboxamide,
(42) N-(4-{[2-({[4-(Dimethylamino)piperidin-1-yl]carbonyl}amino)pyridin-4-yl]oxy}-2,5-difluorophenyl)-N′-(4-fluorophenyl)cyclopropane-1,1-dicarboxamide,
(43) N-{2,5-Difluoro-4-[(2-{[(4-methylpiperazin-1-yl)carbonyl]amino}pyridin-4-yl]oxy}phenyl)-N′-(4-fluorophenyl)cyclopropane-1,1-dicarboxamide,
(44) N-{2,5-Difluoro-4-[(2-{[(4-hydroxypiperidin-1-yl)carbonyl]amino}pyridin-4-yl)oxy]phenyl}-N′-(4-fluorophenyl)cyclopropane-1,1-dicarboxamide,
(45) N-{4-[(2-{[(4-Azetidin-1-ylpiperidin-1-yl)carbonyl]amino}pyridin-4-yl)oxy]oxy}-2,5-difluorophenyl}-N′-(4-fluorophenyl)cyclopropane-1,1-dicarboxamide,
(46) N-(2,5-Difluoro-4-{[2-({[3-(2-dimethylaminoacetoxy)azetidin-1-yl]carbonyl}amino)pyridin-4-yl]oxy}phenyl)-N′-(4-fluorophenyl)cyclopropane-1,1-dicarboxamide,
(47) N-(2,5-Difluoro-4-{[2-({[(3S)-3-hydroxypyrrolidin-1-yl]carbonyl}amino)pyridin-4-yl]oxy}phenyl)-N′-(4-fluorophenyl)cyclopropane-1,1-dicarboxamide or
(48) N-(2,5-Difluoro-4-{[2-({[(3R)-3-hydroxypyrrolidin-1-yl]carbonyl}amino)pyridin-4-yl]oxy}phenyl)-N′-(4-fluorophenyl)cyclopropane-1,1-dicarboxamide.
   [63] A compound represented by the following formula, a salt thereof or a hydrate of the foregoing of [43], wherein a compound represented by the formula (I) is
(1) N-(2-Fluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide
(2) N-[4-({2-[(Azetidin-1-ylcarbonyl)amino]pyridin-4-yl}oxy)-2-fluorophenyl]-N′-(4-fluorophenyl)cyclopropane-1,1-dicarboxamide
(3) N-{2,5-Difluoro-4-[(2-{[(3-hydroxyazetidin-1-yl)carbonyl]amino}pyridin-4-yl)oxy]phenyl}-N′-(4-fluorophenyl)cyclopropane-1,1-dicarboxamide
(4) N-(2,5-Difluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide
(5) N-(2,5-Difluoro-4-{[2-({[methyl(1-methylpiperidin-4-yl)amino]carbonyl}amino)pyridin-4-yl]oxy}phenyl)-N′-(4-fluorophenyl)cyclopropane-1,1-dicarboxamide or
(6) N-(2,5-Difluoro-4-{[2-({[3-(hydroxymethyl)azetidin-1-yl]carbonyl}amino)pyridin-4-yl]oxy}phenyl)-N′-(4-fluorophenyl)cyclopropane-1,1-dicarboxamide.
   [64] Use of a compound represented by the following formula, a salt thereof or a hydrate of the foregoing for treating a disease associated with activation of VEGFR-1, VEGFR-2, VEGFR-3, RON, RET and/or KIT:

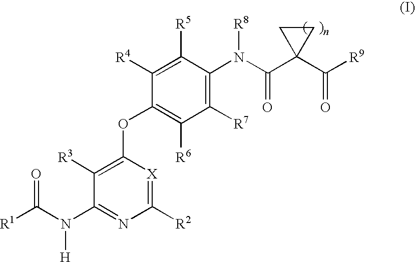

wherein R¹ represents a 3- to 10-membered non-aromatic heterocyclic group wherein the group is limited to a group having nitrogen as a ring constituent atom and the nitrogen having a bonding hand, or a group represented by the formula —NR^{11a}R^{11b}, wherein R^{11a} and R^{11b} may be the same or different and each represents hydrogen, C₁₋₆ alkyl, C₃₋₆ alkenyl, C₃₋₆ alkynyl, C₃₋₁₀ cycloalkyl, C₆₋₁₀ aryl, 5- to 10-membered heteroaryl or a 4- to 10-membered non-aromatic heterocyclic group, and R^{11a} and R^{11b} may be substituted with a substituent selected from Substituent Group A or Substituent Group B and R¹ may be substituted with a substituent selected from Substituent Group A or Substituent Group B;

R² and R³ represent hydrogen;

R⁴, R⁵, R⁶ and R⁷ may be the same or different and each represents hydrogen, halogen, hydroxyl, cyano, trifluoromethyl, C₁₋₆ alkyl, C₂₋₆ alkenyl, C₂₋₆ alkynyl, C₁₋₆ alkoxy, amino, mono-C₁₋₆ alkylamino, di-C₁₋₆ alkylamino or a group represented by the formula —CO—R¹², wherein R¹² represents hydrogen, hydroxyl, C₁₋₆ alkyl, C₁₋₆ alkoxy, amino, mono-C₁₋₆ alkylamino or di-C₁₋₆ alkylamino;

R⁸ represents hydrogen or C₁₋₆ alkyl;

R⁹ represents a 3- to 10-membered non-aromatic heterocyclic group wherein the group is limited to a group having nitrogen as a ring constituent atom and the nitrogen having a bonding hand, or a group represented by the formula —NR^{11a}R^{11b}, wherein R^{11a} and R^{11b} represent the same meaning as described above and R⁹ may be substituted with a substituent selected from Substituent Group A or Substituent Group B;

n represents an integer of 1 or 2; and

X represents a group represented by the formula —C(R¹¹)═ or nitrogen, wherein R¹⁰ represents hydrogen, halogen, cyano, C₁₋₆ alkyl, C₂₋₆ alkenyl, C₂₋₆ alkynyl or a group represented by the formula —CO—R¹², wherein R¹² represents the same meaning as recited above;

wherein Substituent Group A consists of halogen, hydroxyl, mercapto, nitro, cyano and oxo;

wherein Substituent Group B consists of C₁₋₆ alkyl, C₂₋₆ alkenyl, C₂₋₆ alkynyl, C₃₋₁₀ cycloalkyl, C₆- to aryl, 5- to 10-membered heteroaryl, a 3- to 10-membered non-aromatic heterocyclic group, C₁₋₆ alkoxy, C₃₋₆ alkenyloxy, C₃₋₆ alkynyloxy, C₃₋₁₀ cycloalkoxy, C₆₋₁₀ aryloxy, 5- to 10-membered heteroaryloxy, 4- to 10-membered non-aromatic heterocyclicoxy, C₁₋₆ alkylthio, C₃₋₆ alkenylthio, C₃₋₆ alkynylthio, C₃₋₁₀ cycloalkylthio, C₆₋₁₀ arylthio, 5- to 10-membered heteroarylthio, 4- to 10-membered non-aromatic heterocyclicthio and a group represented by the formula -T¹-T²-T³, and each group in Substituent Group B may be substituted with a substituent selected from Substituent Group C, wherein T¹ represents a direct bond or C₁₋₆ alkylene, T² represents carbonyl, sulfinyl, sulfonyl, a group represented by the formula —C(═O)—O—, a group represented by the formula —O—C(═O)—, a group represented by the formula —SO₂—O—, a group represented by the formula —O—SO₂—, a group represented by the formula —NR^{T1}—, a group represented by the formula —C(═O)—NR^{T1}—, a group represented by the formula —NR^{T1}—C(═O)—, a group represented by the formula —SO₂—NR^{T1}— or a group represented by the formula —NR^{T1}—SO₂—, T³ represents hydrogen, C₁₋₆ alkyl, C₃₋₆ alkenyl, C₃₋₆ alkynyl, C₃₋₁₀ cycloalkyl, C₆₋₁₀ aryl, 5- to 10-membered heteroaryl or a 4- to 10-membered non-aromatic heterocyclic group, and R^{T1} represents hydrogen or C₁₋₆ alkyl; and

wherein Substituent Group C consists of halogen, hydroxyl, mercapto, nitro, cyano, oxo, C₁₋₆ alkyl, C₂₋₆ alkenyl, C₂₋₆ alkynyl, C₃₋₁₀ cycloalkyl, C₆₋₁₀ aryl, 5- to 10-membered heteroaryl, a 3- to 10-membered non-aromatic heterocyclic group, C₁₋₆ alkoxy, C₁₋₆ alkylthio, mono-C₁₋₆ alkylamino and di-C₁₋₆ alkylamino.

[65] Use of [64], wherein R¹ represents a 3- to 10-membered non-aromatic heterocyclic group optionally substituted with a substituent selected from Substituent Group A or Substituent Group B recited in [64], wherein the group is limited to a group having nitrogen as a ring constituent atom and the nitrogen having a bonding hand.
[66] Use of [64], wherein R¹ represents a group represented by the formula (II):

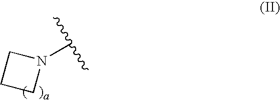

wherein a represents an integer of 1 to 4;
or a group represented by the formula (III):

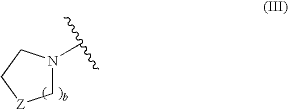

wherein b represents an integer of 1 to 3, and Z represents oxygen, sulfur, carbonyl, sulfonyl, or a group represented by the formula —NR^{Z}—, wherein R^{Z} represents hydrogen or C₁₋₆ alkyl, and the groups represented by the formula (II) or (III) may be substituted with a substituent selected from Substituent Group A or Substituent Group B recited in [64].
[67] Use of [64], wherein R¹ represents azetidin-1-yl optionally substituted with a substituent selected from Substituent Group D, pyrrolidin-1-yl optionally substituted with a substituent selected from Substituent Group D, piperidin-1-yl optionally substituted with a substituent selected from Substituent Group D, azepan-1-yl optionally substituted with a substituent selected from Substituent Group D, piperazin-1-yl optionally substituted with a substituent selected from Substituent Group D, diazepan-1-yl optionally substituted with a substituent selected from Substituent Group D, morpholin-4-yl optionally substituted with a substituent selected from Substituent Group D, thiomorpholin-4-yl optionally substituted with a substituent selected from Substituent Group D, 1,1-dioxothiomorpholin-4-yl optionally substituted with a substituent selected from Substituent Group D,

wherein Substituent Group D consists of halogen, hydroxyl, mercapto, cyano, formyl, oxo, C₁₋₆ alkyl, C₃₋₁₀ cycloalkyl, C₁₋₆ alkoxy, amino, mono-C₁₋₆ alkylamino, di-C₁₋₆ alkylamino, azetidinyl, pyrrolidinyl, piperidinyl, piperazinyl, diazepanyl and a group represented by -T⁴-T⁵, wherein T⁴ represents carbonyl or sulfonyl, and T⁵ represents C₁₋₆ alkyl, C₃₋₁₀ cycloalkyl, azetidinyl, pyrrolidinyl, piperidinyl, hydroxyl, C₁₋₆ alkoxy, amino, mono-C₁₋₆ alkylamino or di-C₁₋₆ alkylamino,

where each group included in Substituent Group D may be substituted with hydroxyl, C₁₋₆ alkyl, di-C₁₋₆ alkylamino, azetidinyl or pyrrolidinyl.

[68] Use of [64], wherein R¹ represent azetidin-1-yl optionally substituted with a substituent selected from Substituent Group E, pyrrolidin-1-yl optionally substituted with a substituent selected from Substituent Group E, piperidin-1-yl optionally substituted with a substituent selected from Substituent Group E, piperazin-1-yl optionally substituted with a substituent selected from Substituent Group E, diazepan-1-yl optionally substituted with a substituent selected from Substituent Group E or morpholin-4-yl optionally substituted with a substituent selected from Substituent Group E,

wherein Substituent Group E consists of methyl, ethyl, dimethylamino, azetidinyl, pyrrolidinyl, piperidinyl and piperazinyl,

where each group included in Substituent Group E may be substituted with hydroxyl, methyl, dimethylamino, azetidinyl, pyrrolidinyl or piperidinyl.

[69] Use of [64], wherein R¹ represents azetidin-1-yl optionally substituted with a substituent selected from Substituent Group G, pyrrolidin-1-yl optionally substituted with a substituent selected from Substituent Group G, piperidin-1-yl optionally substituted with a substituent selected from Substituent Group G or piperazin-1-yl optionally substituted with a substituent selected from Substituent Group G,

wherein Substituent Group G consists of dimethylamino, azetidinyl, pyrrolidinyl, piperidinyl, piperazinyl, dimethylaminomethyl, dimethylaminoethyl, azetidin-1-ylmethyl, pyrrolidin-1-ylmethyl and piperidin-1-ylmethyl,

where each group included in Substituent Group G may be substituted with methyl or dimethylamino.

[69-1] Use of [64], wherein R¹ represents azetidin-1-yl optionally substituted with a substituent selected from Substituent Group G-1, pyrrolidin-1-yl optionally substituted with a substituent selected from Substituent Group G-1, piperidin-1-yl optionally substituted with a substituent selected from Substituent Group G-1 or piperazin-1-yl optionally substituted with a substituent selected from Substituent Group G-1,

wherein Substituent Group G-1 consists of azetidinyl, pyrrolidinyl, piperidinyl, piperazinyl, dimethylaminomethyl, dimethylaminoethyl, azetidin-1-ylmethyl, pyrrolidin-1-ylmethyl and piperidin-1-ylmethyl,

where each group included in Substituent Group G-1 may be substituted with methyl or dimethylamino.

[69-2] Use of [64], wherein R¹ represents azetidin-1-yl having dimethylamino, pyrrolidin-1-yl having dimethylamino or piperidin-1-yl having dimethylamino.
[69-3] Use of [64], wherein R¹ represents azetidin-1-yl optionally substituted with a substituent selected from Substituent Group G-2, pyrrolidin-1-yl substituted with a substituent selected from Substituent Group G-2 or piperidin-1-yl substituted with a substituent selected from Substituent Group G-2,

wherein Substituent Group G-2 consists of hydroxyl, methoxy, hydroxymethyl and dimethylaminoacetoxy.

[69-4] Use of [64], wherein R¹ represents [2-(dimethylamino)ethyl]piperazin-1-yl, 4-pyrrolidin-1-ylpiperidin-1-yl, 4-[(dimethylamino)methyl]piperidin-1-yl, 4-azetidin-1-ylpiperidin-1-yl, 4-[3-(dimethylamino)azetidin-1-yl]piperidin-1-yl, 4-(4-methylpiperazin-1-yl)piperidin-1-yl, 4-(1-methylpiperidin-4-yl)piperazin-1-yl, 4-(1-methylazetidin-3-yl)piperazin-1-yl, 4-(dimethylamino)piperidin-1-yl, 4-(azetidin-1-ylmethyl)piperidin-1-yl, 4-(pyrrolidin-1-ylmethyl)piperidin-1-yl, (3S)-3-(dimethylamino)pyrrolidin-1-yl, (3R)-3-(dimethylamino)pyrrolidin-1-yl, azetidin-1-yl, pyrrolidin-1-yl, morpholin-4-yl, 4-methylpiperazin-1-yl, 3-hydroxyazetidin-1-yl, 1,3′-biazetidin-1′-yl, 3-(hydroxymethyl)azetidin-1-yl, 3-(dimethylamino) azetidin-1-yl, 3-[(dimethylamino)methyl]azetidin-1-yl, 4-hydroxypiperidin-1-yl, 4-(hydroxymethyl)piperidin-1-yl, (3R)-3-hydroxypyrrolidin-1-yl, (3S)-3-hydroxypyrrolidin-1-yl, 3-(azetidin-1-ylmethyl)azetidin-1-yl or 3-(2-dimethylaminoacetoxy)azetidin-1-yl.
[70] Use of [64], wherein R¹ represents a group represented by the formula —NR^{11a}R^{11b}, wherein R^{11a} and R^{11b} represent the same meaning as recited in [64].
[71] Use of [64], wherein R¹ represents a group represented by the formula —NR^{11c}R^{11d}, wherein R^{11c} represents hydrogen or C₁₋₆ alkyl, and R^{11d} represents C₁₋₆ alkyl or a group represented by the formula (IV):

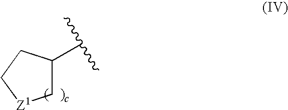

wherein c represents an integer of 1 to 3, and Z¹ represents oxygen, sulfur, carbonyl, sulfonyl or a group represented by the formula —NR^{Z1}, wherein R^{Z1} represents hydrogen or C₁₋₆ alkyl, and R^{11d} may be substituted with a substituent selected from Substituent Group A or Substituent Group B recited in [64].
[72] Use of [64], wherein R¹ represents a group represented by the formula —NR^{11e}R^{11f}, wherein R^{11e} represents hydrogen or C₁₋₆ alkyl, and R^{11f} represents C₁₋₆ alkyl, pyrrolidin-3-yl, piperidin-3-yl, piperidin-4-yl or tetrahydropyran-4-yl, and R^{11f} may be substituted with a substituent selected from Substituent Group D recited in [67].
[73] Use of [64], wherein R¹ represents a group represented by the formula —NR^{11g}R^{11h}, wherein R^{11g} represents hydrogen or methyl, and R^{11h} represents n-propyl, n-butyl, pyrrolidin-3-yl, piperidin-3-yl, piperidin-4-yl or tetrahydropyran-4-yl, and R^{11h} may be substituted with a substituent selected from Substituent Group F,

wherein Substituent Group F consists of methyl, ethyl, n-propyl, acetyl, dimethylamino, diethylamino, azetidinyl, pyrrolidinyl and piperazinyl,

where each group included in Substituent Group F may be substituted with methyl or dimethylamino.

[74] Use of [64], wherein R¹ represents a group represented by the formula —N(CH₃)R¹¹ⁱ, wherein R¹¹ⁱ represents n-propyl, n-butyl, pyrrolidin-3-yl or piperidin-4-yl, and R¹¹ⁱ may be substituted with a substituent selected from Substituent Group H,

wherein Substituent Group H consists of dimethylamino, diethylamino, dimethylaminoethyl, dimethylaminopropyl and 1-methylazetidin-3-yl.

[75] Use of [64], wherein R¹ represents a group represented by the formula —N(CH₃)R^{11j}, wherein R^{11j} represents 1-methylpiperidin-4-yl or 1-ethylpiperidin-4-yl.
[75-1] Use of [64], wherein R¹ represents a group represented by the formula —N(CH₃)R^{11k}, wherein R^{11k} represents 3-(dimethylamino)propyl or 1-[2-(dimethylamino) ethyl]piperidin-4-yl.
[75-2] Use of [64], wherein R¹ represents methyl(1-methylpiperidin-4-yl)amino, (1-ethylpiperidin-4-yl)(methyl)amino, [3-(dimethylamino)propyl](methyl)amino or {1-[2-(dimethylamino)ethyl]piperidin-4-yl}(methyl)amino.
[76] Use of any one of [64] to [75-2], wherein R⁴, R⁵, R⁶ and R⁷ may be the same or different and each represents hydrogen, halogen or C₁₋₆ alkyl.
[77] Use of any one of [64] to [76], wherein R⁸ represents hydrogen.
[78] Use of any one of [64] to [77], wherein X represents a group represented by the formula —C(R^{10a})=, wherein R^{10a} represents hydrogen, halogen or cyano.
[79] Use of any one of [64] to [77], wherein X represents nitrogen.
[80] Use of any one of [64] to [79], wherein n represents 1.
[81] Use of any one of [64] to [80], wherein R⁹ represents mono-C₁₋₆ alkylamino optionally substituted with a substituent selected from Substituent Group A or Substituent Group B recited in [64], mono-C₃₋₁₀ cycloalkylamino optionally substituted with a substituent selected from Substituent Group A or Substituent Group B recited in [64], mono-C₆₋₁₀ arylamino optionally substituted with a substituent selected from Substituent Group A or Substituent Group B recited in [64], mono-5- to 10-membered heteroarylamino optionally substituted with a substituent selected from Substituent Group A or Substituent Group B recited in [64] or mono-4- to 10-membered non-aromatic heterocyclic amino optionally substituted with a substituent selected from Substituent Group A or Substituent Group B recited in [64].
[82] Use of any one of [64] to [80], wherein R⁹ represents mono-C₃₋₁₀ cycloalkylamino optionally substituted with a substituent selected from Substituent Group A or Substituent Group B recited in [1] or mono-C₆₋₁₀ arylamino optionally substituted with a substituent selected from Substituent Group A or Substituent Group B recited in [1].
[82-1] Use of any one of [64] to [80], wherein R⁹ represents mono-C₃₋₁₀ cycloalkylamino optionally substituted with a substituent selected from Substituent Group I or mono-C₆₋₁₀ arylamino optionally substituted with a substituent selected from Substituent Group I,

wherein Substituent Group I consists of halogen, trifluoromethyl, cyano, C₁₋₆ alkyl and C₁₋₆ alkoxy.

[82-2] Use of any one of [64] to [80], wherein R⁹ represents cyclopentylamino optionally substituted with a substituent selected from Substituent Group I recited in [82-1], cyclohexylamino optionally substituted with a substituent selected from Substituent Group I recited in [82-1], cycloheptylamino optionally substituted with a substituent selected from Substituent Group I recited in [82-1] or phenylamino optionally substituted with a substituent selected from Substituent Group I recited in [82-1].
[83] Use of [64], wherein a compound represented by the formula (I) is
(1) N-[4-({2-[({4-[2-(Dimethylamino)ethyl]piperazin-1-yl}carbonyl)amino]pyridin-4-yl}oxy)-2-fluorophenyl]-N′-(4-fluorophenyl)cyclopropane-1,1-dicarboxamide,
(2) N-(2-Fluoro-4-{[2-({[methyl(1-methylpiperidin-4-yl)amino]carbonyl}amino)pyridin-4-yl]oxy}phenyl)-N′-(4-fluorophenyl)cyclopropane-1,1-dicarboxamide,
(3) N-(4-Fluorophenyl)-N′-{2-fluoro-4-[(2-{[(4-pyrrolidin-1-ylpiperidin-1-yl)carbonyl]amino}pyridin-4-yl)oxy]phenyl}cyclopropane-1,1-dicarboxamide,
(4) N-[4-({2-[({4-[(Dimethylamino)methyl]piperidin-1-yl}carbonyl)amino]pyridin-4-yl}oxy)-2-fluorophenyl]-N′-(4-fluorophenyl)cyclopropane-1,1-dicarboxamide,
(5) N-{4-[(2-{[(4-Azetidin-1-ylpiperidin-1-yl)carbonyl]amino}pyridin-4-yl)oxy]-2-fluorophenyl}-N′-(4-fluorophenyl)cyclopropane-1,1-dicarboxamide,
(6) N-[4-({2-[({4-[3-(Dimethylamino)azetidin-1-yl]piperidin-1-yl}carbonyl)amino]pyridin-4-yl}oxy)-2-fluorophenyl]-N′-(4-fluorophenyl)cyclopropane-1,1-dicarboxamide,
(7) N-(2-Fluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide,
(8) N-(2-Fluoro-4-{[2-({[4-(1-methylpiperidin-4-yl)piperazin-1-yl]carbonyl}amino)pyridin-4-yl]oxy}phenyl)-N′-(4-fluorophenyl)cyclopropane-1,1-dicarboxamide,
(9) N-(2-Fluoro-4-{[2-({[4-(1-methylazetidin-3-yl)piperazin-1-yl]carbonyl}amino)pyridin-4-yl]oxy}phenyl)-N′-(4-fluorophenyl)cyclopropane-1,1-dicarboxamide,
(10) N-(4-{[2-({[4-(Dimethylamino)piperidin-1-yl]carbonyl}amino)pyridin-4-yl]oxy}-2-fluorophenyl)-N′-(4-fluorophenyl)cyclopropane-1,1-dicarboxamide,
(11) N-(4-{[2-({[4-(Azetidin-1-ylmethyl)piperidin-1-yl]carbonyl}amino)pyridin-4-yl]oxy}-2-fluorophenyl)-N′-(4-fluorophenyl)cyclopropane-1,1-dicarboxamide,
(12) N-(4-Fluorophenyl)-N′-(2-fluoro-4-{[2-({[4-(pyrrolidin-1-ylmethyl)piperidin-1-yl]carbonyl}amino)pyridin-4-yl]oxy}phenyl)cyclopropane-1,1-dicarboxamide,
(13) N-(4-{[2-({[(3S)-3-(Dimethylamino)pyrrolidin-1-yl]carbonyl}amino)pyridin-4-yl]oxy}-2-fluorophenyl)-N′-(4-fluorophenyl)cyclopropane-1,1-dicarboxamide,
(14) N-(4-{[2-({[(3R)-3-(Dimethylamino)pyrrolidin-1-yl]carbonyl}amino)pyridin-4-yl]oxy}-2-fluorophenyl)-N′-(4-fluorophenyl)cyclopropane-1,1-dicarboxamide,
(15) N-(2-Fluoro-4-{[2-({[methyl(1-methylpiperidin-4-yl)amino]carbonyl}amino)pyridin-4-yl]oxy}phenyl)-N′-phenylcyclopropane-1,1-dicarboxamide,
(16) N-(2-Fluoro-4-{[2-({[4-(4-methylpiperazin-1-yl)piperidin-1-yl]carbonyl}amino)pyridin-4-yl]oxy}phenyl)-N′-phenylcyclopropane-1,1-dicarboxamide,
(17) N-[4-({2-[({4-[3-(Dimethylamino)azetidin-1-yl]piperidin-1-yl}carbonyl)amino]pyridin-4-yl}oxy)-2-fluorophenyl]-N′-phenylcyclopropane-1,1-dicarboxamide,
(18) N-(4-{[2-({[(1-Ethylpiperidin-4-yl)(methyl)amino]carbonyl}amino)pyridin-4-yl]oxy}-2-fluorophenyl)-N′-phenylcyclopropane-1,1-dicarboxamide,
(19) N-[4-({2-[(Azetidin-1-ylcarbonyl)amino]pyridin-4-yl}oxy)-2-fluorophenyl]-N′-(4-fluorophenyl)cyclopropane-1,1-dicarboxamide,
(20) N-(4-Fluorophenyl)-N′-[2-fluoro-4-({2-[(pyrrolidin-1-ylcarbonyl)amino]pyridin-4-yl}oxy)phenyl]cyclopropane-1,1-dicarboxamide,
(21) N-{2-Fluoro-4-[(2-{[(3-hydroxyazetidin-1-yl)carbonyl]amino}pyridin-4-yl)oxy]phenyl}-N′-(4-fluorophenyl)cyclopropane-1,1-dicarboxamide,
(22) N-[4-({2-[(1,3′-Biazetidin-1′-ylcarbonyl)amino]pyridin-4-yl}oxy)-2-fluorophenyl]-N′-(4-fluorophenyl)cyclopropane-1,1-dicarboxamide,
(23) N-(2-Fluoro-4-{[2-({[3-(hydroxymethyl)azetidin-1-yl]carbonyl}amino)pyridin-4-yl]oxy}phenyl)-N′-(4-fluorophenyl)cyclopropane-1,1-dicarboxamide,
(24) N-(4-{[2-({[3-(Dimethylamino)azetidin-1-yl]carbonyl}amino)pyridin-4-yl]oxy}-2-fluorophenyl)-N′-(4-fluorophenyl)cyclopropane-1,1-dicarboxamide,
(25) N-[4-({2-[({3-[(Dimethylamino)methyl] azetidin-1-yl}carbonyl)amino]pyridin-4-yl}oxy)-2-fluorophenyl]-N′-(4-fluorophenyl)cyclopropane-1,1-dicarboxamide,
(26) N-{2-Fluoro-4-[(2-{[(4-hydroxypiperidin-1-yl)carbonyl]amino}pyridin-4-yl)oxy]phenyl}-N′-(4-fluorophenyl)cyclopropane-1,1-dicarboxamide,
(27) N-(2-Fluoro-4-{[2-({[4-(hydroxymethyl)piperidin-1-yl]carbonyl}amino)pyridin-4-yl]oxy}phenyl)-N′-(4-fluorophenyl)cyclopropane-1,1-dicarboxamide,
(28) N-(2-Fluoro-4-{[2-({[(3R)-3-hydroxypyrrolidin-1-yl]carbonyl}amino)pyridin-4-yl]oxy}phenyl)-N′-(4-fluorophenyl)cyclopropane-1,1-dicarboxamide,
(29) N-(2-Fluoro-4-{[2-({[(3S)-3-hydroxypyrrolidin-1-yl]carbonyl}amino)pyridin-4-yl]oxy}phenyl)-N′-(4-fluorophenyl)cyclopropane-1,1-dicarboxamide,
(30) N-[4-({2-[(Azetidin-1-ylcarbonyl)amino]pyridin-4-yl}oxy)-2,5-difluorophenyl]-N′-(4-fluorophenyl)cyclopropane-1,1-dicarboxamide,
(31) N-{2,5-Difluoro-4-[(2-{[(3-hydroxyazetidin-1-yl)carbonyl]amino}pyridin-4-yl)oxy]phenyl}-N′-(4-fluorophenyl)cyclopropane-1,1-dicarboxamide,
(32) N-(2,5-Difluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide,
(33) N-[2,5-Difluoro-4-({2-[({3-[(dimethylamino)methyl] azetidin-1-yl}carbonyl)amino]pyridin-4-yl}oxy)phenyl]-N′-(4-fluorophenyl)cyclopropane-1,1-dicarboxamide,
(34) N-(2,5-Difluoro-4-{[2-({[methyl(1-methylpiperidin-4-yl)amino]carbonyl}amino)pyridin-4-yl]oxy}phenyl)-N′-(4-fluorophenyl)cyclopropane-1,1-dicarboxamide,
(35) N-{4-[(2-{[3-(Azetidin-1-ylmethyl)azetidin-1-ylcarbonyl]amino}pyridin-4-yl)oxy]-2,5-difluorophenyl}-N′-(4-fluorophenyl)cyclopropane-1,1-dicarboxamide,
(36) N-(2,5-Difluoro-4-{[2-({[3-(hydroxymethyl)azetidin-1-yl]carbonyl}amino)pyridin-4-yl]oxy}phenyl)-N′-(4-fluorophenyl)cyclopropane-1,1-dicarboxamide,
(37) N-{2,5-Difluoro-4-[(4-{[(3-hydroxyazetidin-1-yl)carbonyl]amino}pyrimidin-6-yl)oxy]phenyl}-N′-(4-fluorophenyl)cyclopropane-1,1-dicarboxamide,
(38) N-[4-({4-[({3-[(Dimethylamino)methyl] azetidin-1-yl}carbonyl)amino]pyrimidin-6-yl}oxy)-2,5-difluorophenyl]-N′-(4-fluorophenyl)cyclopropane-1,1-dicarboxamide,
(39) N-(2,5-Difluoro-4-{[4-({[3-(hydroxymethyl)azetidin-1-yl]carbonyl}amino)pyrimidin-6-yl]oxy}phenyl)-N′-(4-fluorophenyl)cyclopropane-1,1-dicarboxamide,
(40) N-(2,5-Difluoro-4-{[4-({[methyl(1-methylpiperidin-4-yl)amino]carbonyl}amino)pyrimidin-6-yl]oxy}phenyl)-N′-(4-fluorophenyl)cyclopropane-1,1-dicarboxamide,
(41) N-(2,5-Difluoro-4-{[4-({[4-(4-methylpiperazin-1-yl)piperidin-1-yl]carbonyl}amino)pyrimidin-6-yl]oxy}phenyl)-N′-(4-fluorophenyl)cyclopropane-1,1-dicarboxamide,
(42) N-(4-{[2-({[4-(Dimethylamino)piperidin-1-yl]carbonyl}amino)pyridin-4-yl]oxy}-2,5-difluorophenyl)-N′-(4-fluorophenyl)cyclopropane-1,1-dicarboxamide,
(43) N-{2,5-Difluoro-4-[(2-{[(4-methylpiperazin-1-yl)carbonyl]amino}pyridin-4-yl]oxy}phenyl)-N′-(4-fluorophenyl)cyclopropane-1,1-dicarboxamide,
(44) N-{2,5-Difluoro-4-[(2-{[(4-hydroxypiperidin-1-yl)carbonyl]amino}pyridin-4-yl)oxy]phenyl}-N′-(4-fluorophenyl)cyclopropane-1,1-dicarboxamide,
(45) N-{4-[(2-{[(4-Azetidin-1-ylpiperidin-1-yl)carbonyl]amino}pyridin-4-yl)oxy]oxy}-2,5-difluorophenyl}-N′-(4-fluorophenyl)cyclopropane-1,1-dicarboxamide,
(46) N-(2,5-Difluoro-4-{[2-({[3-(2-dimethylaminoacetoxy)azetidin-1-yl]carbonyl}amino)pyridin-4-yl]oxy}phenyl)-N′-(4-fluorophenyl)cyclopropane-1,1-dicarboxamide,
(47) N-(2,5-Difluoro-4-{[2-({[(3S)-3-hydroxypyrrolidin-1-yl]carbonyl}amino)pyridin-4-yl]oxy}phenyl)-N′-(4-fluorophenyl)cyclopropane-1,1-dicarboxamide or
(48) N-(2,5-Difluoro-4-{[2-({[(3R)-3-hydroxypyrrolidin-1-yl]carbonyl}amino)pyridin-4-yl]oxy}phenyl)-N′-(4-fluorophenyl)cyclopropane-1,1-dicarboxamide.
   [84] Use of [64], wherein a compound represented by the formula (I) is
(1) N-(2-Fluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide
(2) N-[4-({2-[(Azetidin-1-ylcarbonyl)amino]pyridin-4-yl}oxy)-2-fluorophenyl]-N′-(4-fluorophenyl)cyclopropane-1,1-dicarboxamide
(3) N-{2,5-Difluoro-4-[(2-{[(3-hydroxyazetidin-1-yl)carbonyl]amino}pyridin-4-yl)oxy]phenyl}-N′-(4-fluorophenyl)cyclopropane-1,1-dicarboxamide
(4) N-(2,5-Difluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide
(5) N-(2,5-Difluoro-4-{[2-({[methyl(1-methylpiperidin-4-yl)amino]carbonyl}amino)pyridin-4-yl]oxy}phenyl)-N′-(4-fluorophenyl)cyclopropane-1,1-dicarboxamide or
(6) N-(2,5-Difluoro-4-{[2-({[3-(hydroxymethyl)azetidin-1-yl]carbonyl}amino)pyridin-4-yl]oxy}phenyl)-N′-(4-fluorophenyl)cyclopropane-1,1-dicarboxamide.
   [85] Use of a compound represented by the following formula, a salt thereof or a hydrate of the foregoing for the preparation of a therapeutic agent for a disease associated with activation of VEGFR-1, VEGFR-2, VEGFR-3, RON, RET and/or KIT:

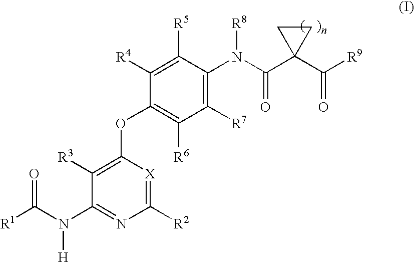

wherein R¹ represents a 3- to 10-membered non-aromatic heterocyclic group wherein the group is limited to a group having nitrogen as a ring constituent atom and the nitrogen having a bonding hand, or a group represented by the formula —NR^{11a}R^{11b}, wherein R^{11a} and R^{11b} may be the same or different and each represents hydrogen, C₁₋₆ alkyl, C₃₋₆ alkenyl, C₃₋₆ alkynyl, C₃₋₁₀ cycloalkyl, C₆₋₁₀ aryl, 5- to 10-membered heteroaryl or a 4- to 10-membered non-aromatic heterocyclic group, and R^{11a} and R^{11b} may be substituted with a substituent selected from Substituent Group A or Substituent Group B and R¹ may be substituted with a substituent selected from Substituent Group A or Substituent Group B;

R² and R³ represent hydrogen;

R⁴, R⁵, R⁶ and R⁷ may be the same or different and each represents hydrogen, halogen, hydroxyl, cyano, trifluoromethyl, C₁₋₆ alkyl, C₂₋₆ alkenyl, C₂₋₆ alkynyl, C₁₋₆ alkoxy, amino, mono-C₁₋₆ alkylamino, di-C₁₋₆ alkylamino or a group represented by the formula —CO—R¹², wherein R¹² represents hydrogen, hydroxyl, C₁₋₆ alkyl, C₁₋₆ alkoxy, amino, mono-C₁₋₆ alkylamino or di-C₁₋₆ alkylamino;

R⁸ represents hydrogen or C₁₋₆ alkyl;

R⁹ represents a 3- to 10-membered non-aromatic heterocyclic group wherein the group is limited to a group having nitrogen as a ring constituent atom and the nitrogen having a bonding hand, or a group represented by the formula —NR^{11a}R^{11b}, wherein R^{11a} and Rub represent the same meaning as described above and R⁹ may be substituted with a substituent selected from Substituent Group A or Substituent Group B;

n represents an integer of 1 or 2; and

X represents a group represented by the formula —C(R¹⁰)═ or nitrogen, wherein R¹⁰ represents hydrogen, halogen, cyano, C₁₋₆ alkyl, C₂₋₆ alkenyl, C₂₋₆ alkynyl or a group represented by the formula —CO—R¹² wherein R¹² represents the same meaning as recited above;

wherein Substituent Group A consists of halogen, hydroxyl, mercapto, nitro, cyano and oxo;

wherein Substituent Group B consists of C₁₋₆ alkyl, C₂₋₆ alkenyl, C₂₋₆ alkynyl, C₃₋₁₀ cycloalkyl, C₆₋₁₀ aryl, 5- to 10-membered heteroaryl, a 3- to 10-membered non-aromatic heterocyclic group, C₁₋₆ alkoxy, C₃₋₆ alkenyloxy, C₃₋₆ alkynyloxy, C₃₋₁₀ cycloalkoxy, C₆₋₁₀ aryloxy, 5- to 10-membered heteroaryloxy, 4- to 10-membered non-aromatic heterocyclicoxy, C₁₋₆ alkylthio, C₃₋₆ alkenylthio, C₃₋₆ alkynylthio, C₃₋₁₀ cycloalkylthio, C₆₋₁₀ arylthio, 5- to 10-membered heteroarylthio, 4- to 10-membered non-aromatic heterocyclicthio and a group represented by the formula -T¹-T²-T³, and each group in Substituent Group B may be substituted with a substituent selected from Substituent Group C, wherein T¹ represents a direct bond or C₁₋₆ alkylene, T² represents carbonyl, sulfinyl, sulfonyl, a group represented by the formula —C(═O)—O—, a group represented by the formula —O—C(═O)—, a group represented by the formula —SO₂—O—, a group represented by the formula —O—SO₂—, a group represented by the formula —NR^{T1}—, a group represented by the formula —C(═O)—NR^{T1}—, a group represented by the formula —NR^{T1}—C(═O)—, a group represented by the formula —SO₂—NR^{T1}— or a group represented by the formula —NR^{T1}—SO₂—, T³ represents hydrogen, C₁₋₆ alkyl, C₃₋₆ alkenyl, C₃₋₆ alkynyl, C₃₋₁₀ cycloalkyl, C₆₋₁₀ aryl, 5- to 10-membered heteroaryl or a 4- to 10-membered non-aromatic heterocyclic group, and R^{T1} represents hydrogen or C₁₋₆ alkyl; and

wherein Substituent Group C consists of halogen, hydroxyl, mercapto, nitro, cyano, oxo, C₁₋₆ alkyl, C₂₋₆ alkenyl, C₂₋₆ alkynyl, C₃₋₁₀ cycloalkyl, C₆₋₁₀ aryl, 5- to 10-membered heteroaryl, a 3- to 10-membered non-aromatic heterocyclic group, C₁₋₆ alkoxy, C₁₋₆ alkylthio, mono-C₁₋₆ alkylamino and di-C₁₋₆ alkylamino.

[86] Use of [85], wherein R¹ represents a 3- to 10-membered non-aromatic heterocyclic group optionally substituted with a substituent selected from Substituent Group A or Substituent Group B recited in [85], wherein the group is limited to a group having nitrogen as a ring constituent atom and the nitrogen having a bonding hand.
[87] Use of [85], wherein R¹ represents a group represented by the formula (II):

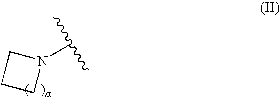

wherein a represents an integer of 1 to 4;
or a group represented by the formula (III):

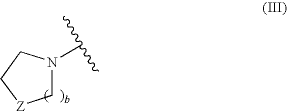

wherein b represents an integer of 1 to 3, and Z represents oxygen, sulfur, carbonyl, sulfonyl, or a group represented by the formula —NR^{Z}, wherein R^{Z} represents hydrogen or C₁₋₆ alkyl, and the groups represented by the formula (II) or (III) may be substituted with a substituent selected from Substituent Group A or Substituent Group B recited in [85].
[88] Use of [85], wherein R¹ represents azetidin-1-yl optionally substituted with a substituent selected from Substituent Group D, pyrrolidin-1-yl optionally substituted with a substituent selected from Substituent Group D, piperidin-1-yl optionally substituted with a substituent selected from Substituent Group D, azepan-1-yl optionally substituted with a substituent selected from Substituent Group D, piperazin-1-yl optionally substituted with a substituent selected from Substituent Group D, diazepan-1-yl optionally substituted with a substituent selected from Substituent Group D, morpholin-4-yl optionally substituted with a substituent selected from Substituent Group D, thiomorpholin-4-yl optionally substituted with a substituent selected from Substituent Group D, 1,1-dioxothiomorpholin-4-yl optionally substituted with a substituent selected from Substituent Group D,

wherein Substituent Group D consists of halogen, hydroxyl, mercapto, cyano, formyl, oxo, C₁₋₆ alkyl, C₃₋₁₀ cycloalkyl, C₁₋₆ alkoxy, amino, mono-C₁₋₆ alkylamino, di-C₁₋₆ alkylamino, azetidinyl, pyrrolidinyl, piperidinyl, piperazinyl, diazepanyl and a group represented by -T⁴-T⁵,

wherein T⁴ represents carbonyl or sulfonyl, and T⁵ represents C₁₋₆ alkyl, C₃₋₁₀ cycloalkyl, azetidinyl, pyrrolidinyl, piperidinyl, hydroxyl, C₁₋₆ alkoxy, amino, mono-C₁₋₆ alkylamino or di-C₁₋₆ alkylamino, where each group included in Substituent Group D may be substituted with hydroxyl, C₁₋₆ alkyl, di-C₁₋₆ alkylamino, azetidinyl or pyrrolidinyl.

[89] Use of [85], wherein R¹ represent azetidin-1-yl optionally substituted with a substituent selected from Substituent Group E, pyrrolidin-1-yl optionally substituted with a substituent selected from Substituent Group E, piperidin-1-yl optionally substituted with a substituent selected from Substituent Group E, piperazin-1-yl optionally substituted with a substituent selected from Substituent Group E, diazepan-1-yl optionally substituted with a substituent selected from Substituent Group E or morpholin-4-yl optionally substituted with a substituent selected from Substituent Group E,

wherein Substituent Group E consists of methyl, ethyl, dimethylamino, azetidinyl, pyrrolidinyl, piperidinyl and piperazinyl,

where each group included in Substituent Group E may be substituted with hydroxyl, methyl, dimethylamino, azetidinyl, pyrrolidinyl or piperidinyl.

[90] Use of [85], wherein R¹ represents azetidin-1-yl optionally substituted with a substituent selected from Substituent Group G, pyrrolidin-1-yl optionally substituted with a substituent selected from Substituent Group G, piperidin-1-yl optionally substituted with a substituent selected from Substituent Group G or piperazin-1-yl optionally substituted with a substituent selected from Substituent Group G,

wherein Substituent Group G consists of dimethylamino, azetidinyl, pyrrolidinyl, piperidinyl, piperazinyl, dimethylaminomethyl, dimethylaminoethyl, azetidin-1-ylmethyl, pyrrolidin-1-ylmethyl and piperidin-1-ylmethyl,

where each group included in Substituent Group G may be substituted with methyl or dimethylamino.

[90-1] Use of [85], wherein R¹ represents azetidin-1-yl optionally substituted with a substituent selected from Substituent Group G-1, pyrrolidin-1-yl optionally substituted with a substituent selected from Substituent Group G-1, piperidin-1-yl optionally substituted with a substituent selected from Substituent Group G-1 or piperazin-1-yl optionally substituted with a substituent selected from Substituent Group G-1,

wherein Substituent Group G-1 consists of azetidinyl, pyrrolidinyl, piperidinyl, piperazinyl, dimethylaminomethyl, dimethylaminoethyl, azetidin-1-ylmethyl, pyrrolidin-1-ylmethyl and piperidin-1-ylmethyl,

where each group included in Substituent Group G-1 may be substituted with methyl or dimethylamino.

[90-2] Use of [85], wherein R¹ represents azetidin-1-yl having dimethylamino, pyrrolidin-1-yl having dimethylamino or piperidin-1-yl having dimethylamino.
[90-3] Use of [85], wherein R¹ represents azetidin-1-yl optionally substituted with a substituent selected from Substituent Group G-2, pyrrolidin-1-yl substituted with a substituent selected from Substituent Group G-2 or piperidin-1-yl substituted with a substituent selected from Substituent Group G-2,

wherein Substituent Group G-2 consists of hydroxyl, methoxy, hydroxymethyl and dimethylaminoacetoxy.

[90-4] Use of [85], wherein R¹ represents [2-(dimethylamino)ethyl]piperazin-1-yl, 4-pyrrolidin-1-ylpiperidin-1-yl, 4-[(dimethylamino)methyl]piperidin-1-yl, 4-azetidin-1-ylpiperidin-1-yl, 4-[3-(dimethylamino)azetidin-1-yl]piperidin-1-yl, 4-(4-methylpiperazin-1-yl)piperidin-1-yl, 4-(1-methylpiperidin-4-yl)piperazin-1-yl, 4-(1-methylazetidin-3-yl)piperazin-1-yl, 4-(dimethylamino)piperidin-1-yl, 4-(azetidin-1-ylmethyl)piperidin-1-yl, 4-(pyrrolidin-1-ylmethyl)piperidin-1-yl, (3S)-3-(dimethylamino)pyrrolidin-1-yl, (3R)-3-(dimethylamino)pyrrolidin-1-yl, azetidin-1-yl, pyrrolidin-1-yl, morpholin-4-yl, 4-methylpiperazin-1-yl, 3-hydroxyazetidin-1-yl, 1,3′-biazetidin-1′-yl, 3-(hydroxymethyl)azetidin-1-yl, 3-(dimethylamino)azetidin-1-yl, 3-[(dimethylamino)methyl]azetidin-1-yl, 4-hydroxypiperidin-1-yl, 4-(hydroxymethyl)piperidin-1-yl, (3R)-3-hydroxypyrrolidin-1-yl, (3S)-3-hydroxypyrrolidin-1-yl, 3-(azetidin-1-ylmethyl)azetidin-1-yl or 3-(2-dimethylaminoacetoxy)azetidin-1-yl.
[91] Use of [85], wherein R¹ represents a group represented by the formula —NR^{11a}R^{11b}, wherein R^{11a} and R^{11b} represent the same meaning as recited in [85].
[92] Use of [85], wherein R¹ represents a group represented by the formula —NR^{11c}R^{11d}, wherein R^{11c} represents hydrogen or C₁₋₆ alkyl, and R^{11d} represents C₁₋₆ alkyl or a group represented by the formula (IV):

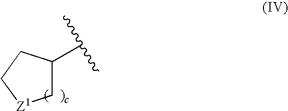

wherein c represents an integer of 1 to 3, and Z¹ represents oxygen, sulfur, carbonyl, sulfonyl or a group represented by the formula —NR^{Z1}, wherein R^{Z1} represents hydrogen or C₁₋₆ alkyl, and R^{11d} may be substituted with a substituent selected from Substituent Group A or Substituent Group B recited in [85].
[93] Use of [85], wherein R¹ represents a group represented by the formula —NR^{11e}R^{11f}, wherein R^{11e} represents hydrogen or C₁₋₆ alkyl, and R^{11f} represents C₁₋₆ alkyl, pyrrolidin-3-yl, piperidin-3-yl, piperidin-4-yl or tetrahydropyran-4-yl, and R^{11f} may be substituted with a substituent selected from Substituent Group D recited in [88].
[94] Use of [85], wherein R¹ represents a group represented by the formula —NR^{11g}R^{11h}, wherein R^{11g} represents hydrogen or methyl, and R^{11h} represents n-propyl, n-butyl, pyrrolidin-3-yl, piperidin-3-yl, piperidin-4-yl or tetrahydropyran-4-yl, and R^{11h} may be substituted with a substituent selected from Substituent Group F,

wherein Substituent Group F consists of methyl, ethyl, n-propyl, acetyl, dimethylamino, diethylamino, azetidinyl, pyrrolidinyl and piperazinyl,

where each group included in Substituent Group F may be substituted with methyl or dimethylamino.

[95] Use of [85], wherein R¹ represents a group represented by the formula —N(CH₃)R¹¹ⁱ, wherein R¹¹ⁱ represents n-propyl, n-butyl, pyrrolidin-3-yl or piperidin-4-yl, and R¹¹ⁱ may be substituted with a substituent selected from Substituent Group H,

wherein Substituent Group H consists of dimethylamino, diethylamino, dimethylaminoethyl, dimethylaminopropyl and 1-methylazetidin-3-yl.

[96] Use of [85], wherein R¹ represents a group represented by the formula —N(CH₃)R^{11j}, wherein R^{11j} represents 1-methylpiperidin-4-yl or 1-ethylpiperidin-4-yl.
[96-1] Use of [85], wherein R¹ represents a group represented by the formula —N(CH₃)R^{11k}, wherein R^{11k} represents 3-(dimethylamino)propyl or 1-[2-(dimethylamino)ethyl]piperidin-4-yl.
[96-2] Use of [85], wherein R¹ represents methyl(1-methylpiperidin-4-yl)amino, (1-ethylpiperidin-4-yl)(methyl)amino, [3-(dimethylamino)propyl](methyl)amino or {1-[2-(dimethylamino)ethyl]piperidin-4-yl}(methyl)amino.
[97] Use of any one of [85] to [96-2], wherein R⁴, R⁵, R⁶ and R⁷ may be the same or different and each represents hydrogen, halogen or C₁₋₆ alkyl.
[98] Use of any one of [85] to [97], wherein R⁸ represents hydrogen.
[99] Use of any one of [85] to [98], wherein X represents a group represented by the formula —C(R^{10a})═, wherein R^{10a} represents hydrogen, halogen or cyano.

[100] Use of any one of [85] to [98], wherein X represents nitrogen.

[101] Use of any one of [85] to [100], wherein n represents 1.

[102] Use of any one of [85] to [101], wherein R⁹ represents mono-C₁₋₆ alkylamino optionally substituted with a substituent selected from Substituent Group A or Substituent Group B recited in [85], mono-C₃₋₁₀ cycloalkylamino optionally substituted with a substituent selected from Substituent Group A or Substituent Group B recited in [85], mono-C₆₋₁₀ arylamino optionally substituted with a substituent selected from Substituent Group A or Substituent Group B recited in [85], mono-5- to 10-membered heteroarylamino optionally substituted with a substituent selected from Substituent Group A or Substituent Group B recited in [85] or mono-4- to 10-membered non-aromatic heterocyclic amino optionally substituted with a substituent selected from Substituent Group A or Substituent Group B recited in [85].

[103] Use of any one of [85] to [101], wherein R⁹ represents mono-C₃₋₁₀ cycloalkylamino optionally substituted with a substituent selected from Substituent Group A or Substituent Group B recited in [85] or mono-C₆₋₁₀ arylamino optionally substituted with a substituent selected from Substituent Group A or Substituent Group B recited in [85].

[103-1] Use of any one of [85] to [101], wherein R⁹ represents mono-C₃₋₁₀ cycloalkylamino optionally substituted with a substituent selected from Substituent Group I or mono-C₆₋₁₀ arylamino optionally substituted with a substituent selected from Substituent Group I,

wherein Substituent Group I consists of halogen, trifluoromethyl, cyano, C₁₋₆ alkyl and C₁₋₆ alkoxy.

[103-2] Use of any one of [85] to [101], wherein R⁹ represents cyclopentylamino optionally substituted with a substituent selected from Substituent Group I recited in [103-1], cyclohexylamino optionally substituted with a substituent selected from Substituent Group I recited in [103-1], cycloheptylamino optionally substituted with a substituent selected from Substituent Group I recited in [103-1] or phenylamino optionally substituted with a substituent selected from Substituent Group I recited in [103-1].

[104] Use of [85], wherein a compound represented by the formula (I) is
(1) N-[4-({2-[({4-[2-(Dimethylamino)ethyl]piperazin-1-yl}carbonyl)amino]pyridin-4-yl}oxy)-2-fluorophenyl]-N′-(4-fluorophenyl)cyclopropane-1,1-dicarboxamide,
(2) N-(2-Fluoro-4-{[2-({[methyl(1-methylpiperidin-4-yl)amino]carbonyl}amino)pyridin-4-yl]oxy}phenyl)-N′-(4-fluorophenyl)cyclopropane-1,1-dicarboxamide,
(3) N-(4-Fluorophenyl)-N′-{2-fluoro-4-[(2-{[(4-pyrrolidin-1-ylpiperidin-1-yl)carbonyl]amino}pyridin-4-yl)oxy]phenyl}cyclopropane-1,1-dicarboxamide,
(4) N-[4-({2-[({4-[(Dimethylamino)methyl]piperidin-1-yl}carbonyl)amino]pyridin-4-yl}oxy)-2-fluorophenyl]-N′-(4-fluorophenyl)cyclopropane-1,1-dicarboxamide,
(5) N-{4-[(2-{[(4-Azetidin-1-ylpiperidin-1-yl)carbonyl]amino}pyridin-4-yl)oxy]-2-fluorophenyl}-N′-(4-fluorophenyl)cyclopropane-1,1-dicarboxamide,
(6) N-[4-({2-[({4-[3-(Dimethylamino)azetidin-1-yl]piperidin-1-yl}carbonyl)amino]pyridin-4-yl}oxy)-2-fluorophenyl]-N′-(4-fluorophenyl)cyclopropane-1,1-dicarboxamide,
(7) N-(2-Fluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide,
(8) N-(2-Fluoro-4-{[2-({[4-(1-methylpiperidin-4-yl)piperazin-1-yl]carbonyl}amino)pyridin-4-yl]oxy}phenyl)-N′-(4-fluorophenyl)cyclopropane-1,1-dicarboxamide,
(9) N-(2-Fluoro-4-{[2-({[4-(1-methylazetidin-3-yl)piperazin-1-yl]carbonyl}amino)pyridin-4-yl]oxy}phenyl)-N′-(4-fluorophenyl)cyclopropane-1,1-dicarboxamide,
(10) N-(4-{[2-({[4-(Dimethylamino)piperidin-1-yl]carbonyl}amino)pyridin-4-yl]oxy}-2-fluorophenyl)-N′-(4-fluorophenyl)cyclopropane-1,1-dicarboxamide,
(11) N-(4-{[2-({[4-(Azetidin-1-ylmethyl)piperidin-1-yl]carbonyl}amino)pyridin-4-yl]oxy}-2-fluorophenyl)-N′-(4-fluorophenyl)cyclopropane-1,1-dicarboxamide,
(12) N-(4-Fluorophenyl)-N′-(2-fluoro-4-{[2-({[4-(pyrrolidin-1-ylmethyl)piperidin-1-yl]carbonyl}amino)pyridin-4-yl]oxy}phenyl)cyclopropane-1,1-dicarboxamide,
(13) N-(4-{[2-({[(3S)-3-(Dimethylamino)pyrrolidin-1-yl]carbonyl}amino)pyridin-4-yl]oxy}-2-fluorophenyl)-N′-(4-fluorophenyl)cyclopropane-1,1-dicarboxamide,
(14) N-(4-{[2-({[(3R)-3-(Dimethylamino)pyrrolidin-1-yl]carbonyl}amino)pyridin-4-yl]oxy}-2-fluorophenyl)-N′-(4-fluorophenyl)cyclopropane-1,1-dicarboxamide,
(15) N-(2-Fluoro-4-{[2-({[methyl(1-methylpiperidin-4-yl)amino]carbonyl}amino)pyridin-4-yl]oxy}phenyl)-N′-phenylcyclopropane-1,1-dicarboxamide,
(16) N-(2-Fluoro-4-{[2-({[4-(4-methylpiperazin-1-yl)piperidin-1-yl]carbonyl}amino)pyridin-4-yl]oxy}phenyl)-N′-phenylcyclopropane-1,1-dicarboxamide,
(17) N-[4-({2-[(f{4-[3-(Dimethylamino)azetidin-1-yl]piperidin-1-yl}carbonyl)amino]pyridin-4-yl}oxy)-2-fluorophenyl]-N′-phenylcyclopropane-1,1-dicarboxamide,
(18) N-(4-{[2-({[(1-Ethylpiperidin-4-yl)(methyl)amino]carbonyl}amino)pyridin-4-yl]oxy}-2-fluorophenyl)-N′-phenylcyclopropane-1,1-dicarboxamide,
(19) N-[4-({2-[(Azetidin-1-ylcarbonyl)amino]pyridin-4-yl}oxy)-2-fluorophenyl]-N′-(4-fluorophenyl)cyclopropane-1,1-dicarboxamide,
(20) N-(4-Fluorophenyl)-N′-[2-fluoro-4-({2-[(pyrrolidin-1-ylcarbonyl)amino]pyridin-4-yl}oxy)phenyl]cyclopropane-1,1-dicarboxamide,
(21) N-{2-Fluoro-4-[(2-{[(3-hydroxyazetidin-1-yl)carbonyl]amino}pyridin-4-yl)oxy]phenyl}-N′-(4-fluorophenyl)cyclopropane-1,1-dicarboxamide,
(22) N-[4-({2-[(1,3′-Biazetidin-1′-ylcarbonyl)amino]pyridin-4-yl}oxy)-2-fluorophenyl]-N′-(4-fluorophenyl)cyclopropane-1,1-dicarboxamide,
(23) N-(2-Fluoro-4-{[2-({[3-(hydroxymethyl)azetidin-1-yl]carbonyl}amino)pyridin-4-yl]oxy}phenyl)-N′-(4-fluorophenyl)cyclopropane-1,1-dicarboxamide,
(24) N-(4-{[2-({[3-(Dimethylamino)azetidin-1-yl]carbonyl}amino)pyridin-4-yl]oxy}-2-fluorophenyl)-N′-(4-fluorophenyl)cyclopropane-1,1-dicarboxamide,
(25) N-[4-({2-[({3-[(Dimethylamino)methyl] azetidin-1-yl}carbonyl)amino]pyridin-4-yl}oxy)-2-fluorophenyl]-N′-(4-fluorophenyl)cyclopropane-1,1-dicarboxamide,
(26) N-{2-Fluoro-4-[(2-{[(4-hydroxypiperidin-1-yl)carbonyl]amino}pyridin-4-yl)oxy]phenyl}-N′-(4-fluorophenyl)cyclopropane-1,1-dicarboxamide,
(27) N-(2-Fluoro-4-{[2-({[4-(hydroxymethyl)piperidin-1-yl]carbonyl}amino)pyridin-4-yl]oxy}phenyl)-N′-(4-fluorophenyl)cyclopropane-1,1-dicarboxamide,
(28) N-(2-Fluoro-4-{[2-({[(3R)-3-hydroxypyrrolidin-1-yl]carbonyl}amino)pyridin-4-yl]oxy}phenyl)-N′-(4-fluorophenyl)cyclopropane-1,1-dicarboxamide,
(29) N-(2-Fluoro-4-{[2-({[(3S)-3-hydroxypyrrolidin-1-yl]carbonyl}amino)pyridin-4-yl]oxy}phenyl)-N′-(4-fluorophenyl)cyclopropane-1,1-dicarboxamide,
(30) N-[4-({2-[(Azetidin-1-ylcarbonyl)amino]pyridin-4-yl}oxy)-2,5-difluorophenyl]-N′-(4-fluorophenyl)cyclopropane-1,1-dicarboxamide,
(31) N-{2,5-Difluoro-4-[(2-{[(3-hydroxyazetidin-1-yl)carbonyl]amino}pyridin-4-yl)oxy]phenyl}-N′-(4-fluorophenyl)cyclopropane-1,1-dicarboxamide,
(32) N-(2,5-Difluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide,
(33) N-[2,5-Difluoro-4-({2-[({3-[(dimethylamino)methyl] azetidin-1-yl}carbonyl)amino]pyridin-4-yl}oxy)phenyl]-N′-(4-fluorophenyl)cyclopropane-1,1-dicarboxamide,
(34) N-(2,5-Difluoro-4-{[2-({[methyl(1-methylpiperidin-4-yl)amino]carbonyl}amino)pyridin-4-yl]oxy}phenyl)-N′-(4-fluorophenyl)cyclopropane-1,1-dicarboxamide,
(35) N-{4-[(2-{[3-(Azetidin-1-ylmethyl)azetidin-1-ylcarbonyl]amino}pyridin-4-yl)oxy]-2,5-difluorophenyl}-N′-(4-fluorophenyl)cyclopropane-1,1-dicarboxamide,
(36) N-(2,5-Difluoro-4-{[2-({[3-(hydroxymethyl)azetidin-1-yl]carbonyl}amino)pyridin-4-yl]oxy}phenyl)-N′-(4-fluorophenyl)cyclopropane-1,1-dicarboxamide,
(37) N-{2,5-Difluoro-4-[(4-{[(3-hydroxyazetidin-1-yl)carbonyl]amino}pyrimidin-6-yl)oxy]phenyl}-N′-(4-fluorophenyl)cyclopropane-1,1-dicarboxamide,
(38) N-[4-({4-[({3-[(Dimethylamino)methyl] azetidin-1-yl}carbonyl)amino]pyrimidin-6-yl}oxy)-2,5-difluorophenyl]-N′-(4-fluorophenyl)cyclopropane-1,1-dicarboxamide,
(39) N-(2,5-Difluoro-4-{[4-({[3-(hydroxymethyl)azetidin-1-yl]carbonyl}amino)pyrimidin-6-yl]oxy}phenyl)-N′-(4-fluorophenyl)cyclopropane-1,1-dicarboxamide,
(40) N-(2,5-Difluoro-4-{[4-({[methyl(1-methylpiperidin-4-yl)amino]carbonyl}amino)pyrimidin-6-yl]oxy}phenyl)-N′-(4-fluorophenyl)cyclopropane-1,1-dicarboxamide,
(41) N-(2,5-Difluoro-4-{[4-({[4-(4-methylpiperazin-1-yl)piperidin-1-yl]carbonyl}amino)pyrimidin-6-yl]oxy}phenyl)-N′-(4-fluorophenyl)cyclopropane-1,1-dicarboxamide,
(42) N-(4-{[2-({[4-(Dimethylamino)piperidin-1-yl]carbonyl}amino)pyridin-4-yl]oxy}-2,5-difluorophenyl)-N′-(4-fluorophenyl)cyclopropane-1,1-dicarboxamide,
(43) N-{2,5-Difluoro-4-[(2-{[(4-methylpiperazin-1-yl)carbonyl]amino}pyridin-4-yl]oxy}phenyl)-N′-(4-fluorophenyl)cyclopropane-1,1-dicarboxamide,
(44) N-{2,5-Difluoro-4-[(2-{[(4-hydroxypiperidin-1-yl)carbonyl]amino}pyridin-4-yl)oxy]phenyl}-N′-(4-fluorophenyl)cyclopropane-1,1-dicarboxamide,
(45) N-{4-[(2-{[(4-Azetidin-1-ylpiperidin-1-yl)carbonyl]amino}pyridin-4-yl)oxy]oxy}-2,5-difluorophenyl}-N′-(4-fluorophenyl)cyclopropane-1,1-dicarboxamide,
(46) N-(2,5-Difluoro-4-{[2-({[3-(2-dimethylaminoacetoxy)azetidin-1-yl]carbonyl}amino)pyridin-4-yl]oxy}phenyl)-N′-(4-fluorophenyl)cyclopropane-1,1-dicarboxamide,
(47) N-(2,5-Difluoro-4-{[2-({[(3S)-3-hydroxypyrrolidin-1-yl]carbonyl}amino)pyridin-4-yl]oxy}phenyl)-N′-(4-fluorophenyl)cyclopropane-1,1-dicarboxamide or
(48) N-(2,5-Difluoro-4-{[2-({[(3R)-3-hydroxypyrrolidin-1-yl]carbonyl}amino)pyridin-4-yl]oxy}phenyl)-N′-(4-fluorophenyl)cyclopropane-1,1-dicarboxamide.

[105] Use of [85], wherein a compound represented by the formula (I) is
(1) N-(2-Fluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide
(2) N-[4-({2-[(Azetidin-1-ylcarbonyl)amino]pyridin-4-yl}oxy)-2-fluorophenyl]-N′-(4-fluorophenyl)cyclopropane-1,1-dicarboxamide
(3) N-{2,5-Difluoro-4-[(2-{[(3-hydroxyazetidin-1-yl)carbonyl]amino}pyridin-4-yl)oxy]phenyl}-N′-(4-fluorophenyl)cyclopropane-1,1-dicarboxamide
(4) N-(2,5-Difluoro-4-{[2-({[4-(4-methylpiperazin-1-yl)piperidin-1-yl]carbonyl}amino)pyridin-4-yl] oxy}phenyl)-N′-(4-fluorophenyl)cyclopropane-1,1-dicarboxamide
(5) N-(2,5-Difluoro-4-{[2-({[methyl(1-methylpiperidin-4-yl)amino]carbonyl}amino)pyridin-4-yl]oxy}phenyl)-N′-(4-fluorophenyl)cyclopropane-1,1-dicarboxamide or
(6) N-(2,5-Difluoro-4-{[2-({[3-(hydroxymethyl)azetidin-1-yl]carbonyl}amino)pyridin-4-yl]oxy}phenyl)-N′-(4-fluorophenyl)cyclopropane-1,1-dicarboxamide.

### EFFECT OF THE INVENTION

The compound of the present invention has inhibitory action against receptor tyrosine kinase of VEGFR-1, VEGFR-2, VEGFR-3, RON, RET and KIT.

### DESCRIPTION OF THE PREFERRED EMBODIMENTS

The symbols and terms as used herein will be defined and the present invention will be described in details below.

Several of the structural formulas for the compounds throughout the present specification represent only one isomeric form for convenience, but the invention encompasses any and all of the geometric isomers as well as optical isomers based on asymmetric carbons, stereoisomers and tautomers, and mixtures of those isomers, which are implied by the structures of the compounds, without being limited to any of the formulas shown for convenience. The compounds of the invention therefore include all those having asymmetric carbons therein and existing in optically active or racemic form, with no particular restrictions on the invention. There are also no restrictions when polymorphic crystalline forms thereof exist, and the compounds may be in one crystalline form or a mixture of different crystalline forms, while anhydrates and hydrates of the compounds of the invention are also included.

The so-called metabolite, a compound which a compound according to the present invention is metabolized in a living body through oxidation, reduction, hydrolysis, conjugation and the others to provide, and the so-called prodrug, a compound which is metabolized in a living body through oxidation, reduction, hydrolysis, conjugation and the others to provide a compound according to the present invention, are also included within the claimed scope of the present invention.

The “salt” includes a salt of an inorganic acid, a salt of an organic acid, a salt of an inorganic base, a salt of an organic base and a salt of an acidic or basic amino acid, among them, a pharmacologically acceptable salt is preferable.

The preferable salt of an inorganic acid includes, for example, a salt of hydrochloric acid, hydrobromic acid, sulfuric acid, nitric acid and phosphoric acid. The preferable salt of an organic acid includes, for example, a salt of acetic acid, succinic acid, fumaric acid, maleic acid, tartaric acid, citric acid, lactic acid, stearic acid, benzoic acid, methanesulfonic acid, ethanesulfonic acid, and p-toluenesulfonic acid.

The preferable salt of an inorganic base includes, for example, an alkali metal salt such as sodium salt and potassium salt, an alkali earth metal salt such as calcium salt and magnesium salt, aluminum salt, and ammonium salt. The preferable salt of an organic base includes, for example, a salt of diethylamine, diethanolamine, meglumine, and N,N-dibenzylethylenediamine.

The preferable salt of an acidic amino acid includes, for example, a salt of aspartic acid and glutamic acid. The preferable salt of a basic amino acid includes, for example, a salt of arginine, lysine and ornithine.

The “halogen” represents fluorine, chlorine, bromine or iodine.

The “C₁₋₆ alkyl” represents an alkyl of straight or branched chain having a carbon number of 1 to 6, and includes, for specific example, methyl, ethyl, 1-propyl (n-propyl), 2-propyl (i-propyl), 2-methyl-1-propyl (i-butyl), 2-methyl-2-propyl (t-butyl), 1-butyl (n-butyl), 2-butyl (s-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,3-dimethyl-1-butyl, 3,3-dimethyl-1-butyl, 2,2-dimethyl-1-butyl, 2-ethyl-1-butyl, 3,3-dimethyl-2-butyl, and 2,3-dimethyl-2-butyl.

The “C₂₋₆ alkenyl” represents an alkenyl of straight or branched chain having one double bond and a carbon number of 2 to 6, and includes, for specific example, ethenyl (vinyl), 1-propenyl, 2-propenyl (allyl), 1-butenyl, 2-butenyl, 3-butenyl, pentenyl, and hexenyl.

The “C₃₋₆ alkenyl” represents an alkenyl of straight or branched chain having one double bond and a carbon number of 3 to 6, and includes, for specific example, 2-propenyl (allyl), 2-butenyl, 3-butenyl, pentenyl, and hexenyl.

The “C₂₋₆ alkynyl” represents an alkynyl of straight or branched chain having one triple bond and a carbon number of 2 to 6, and includes, for specific example, ethynyl, 1-propynyl, 2-propynyl, 1-butynyl, 2-butynyl, 3-butynyl, pentynyl, and hexynyl.

The “C₃₋₆ alkynyl” represents an alkynyl of straight or branched chain having one triple bond and a carbon number of 3 to 6, and includes, for specific example, 2-propynyl, 2-butynyl, 3-butynyl, pentynyl, and hexynyl.

The “C₁₋₆ alkylene” represents a divalent group derived by eliminating further any one hydrogen from the “C₁₋₆ alkyl” defined above, and includes, for specific example, methylene, 1,2-ethylene, 1,1-ethylene, 1,3-propylene, tetramethylene, pentamethylene, and hexamethylene.

The “C₃₋₁₀ cycloalkyl” represents a mono- or di-cyclic saturated aliphatic hydrocarbon group having a carbon number of 3 to 10, and includes, for specific example, cyclopropyl, cyclobutyl, cyclopentyl, cyclohexyl, cycloheptyl, cyclooctyl, cyclononyl, cyclodecyl, bicyclo[2.1.0]pentyl, bicyclo[3.1.0]hexyl, bicyclo [2.1.1]hexyl, bicyclo[4.1.0]heptyl, bicyclo[2.2.1]heptyl (norbornyl), bicyclo[3.3.0]octyl, bicyclo[3.2.1]octyl, bicyclo[2.2.2]octyl, bicyclo[4.3.0]nonyl, bicyclo[3.3.1]nonyl, bicyclo[4.4.0]decyl (decalyl), and bicyclo[3.3.2]decyl.

The “C₆₋₁₀ aryl” represents an aromatic hydrocarbon ring group having a carbon number of 6 to 10, and includes, for specific example, phenyl, 1-naphthyl, 2-naphthyl, indenyl, azulenyl, and heptalenyl.

The “heteroatom” represents nitrogen, oxygen, or sulfur.

The “5- to 10-membered heteroaryl” represents an aromatic ring group having 5 to 10 atoms forming the ring and containing 1 to 5 heteroatoms, and includes, for specific example, furyl, thienyl, pyrrolyl, imidazolyl, triazolyl, tetrazolyl, thiazolyl, pyrazolyl, oxazolyl, isoxazolyl, isothiazolyl, furazanyl, thiadiazolyl, oxadiazolyl, pyridyl, pyrazinyl, pyridazinyl, pyrimidinyl, triazinyl, purinyl, pteridinyl, quinolyl, isoquinolyl, naphthylidinyl, quinoxalinyl, cinnolinyl, quinazolinyl, phthalazinyl, imidazopyridyl, imidazothiazolyl, imidazoxazolyl, benzothiazolyl, benzoxazolyl, benzimidazolyl, indolyl, isoindolyl, indazolyl, pyrrolopyridyl, thienopyridyl, furopyridyl, benzothiadiazolyl, benzoxadiazolyl, pyridopyrimidinyl, benzofuryl, benzothienyl, and thienofuryl.

The preferable example of the “5- to 10-membered heteroaryl” includes furyl, thienyl, pyrrolyl, imidazolyl, thiazolyl, pyrazolyl, oxazolyl, isoxazolyl, isothiazolyl, pyridyl, and pyrimidinyl.

The “3- to 10-membered non-aromatic heterocyclic group” represents

(1) a monocyclic or a bicyclic non-aromatic heterocyclic group
(2) having 3 to 10 atoms in the ring,
(3) containing 1 to 2 heteroatoms among the atoms of the ring,
(4) optionally containing 1 to 2 double bonds in the ring,
(5) optionally containing 1 to 3 carbonyl, sulfinyl, or sulfonyl in the ring.

If the group contains nitrogen in the ring, the nitrogen may have a bond not participating in the formation of the ring. The group includes, for specific example, aziridinyl, azetidinyl, pyrrolidinyl, piperidinyl, azepanyl, azocanyl, piperazinyl, diazepanyl, diazocanyl, diazabicyclo[2.2.1]heptyl, morpholinyl, thiomorpholinyl, 1,1-dioxothiomorpholinyl, oxiranyl, oxetanyl, tetrahydrofuryl, tetrahydropyranyl, dioxanyl, tetrahydrothienyl, tetrahydrothiopyranyl, oxazolidinyl, and thiazolidinyl.

The preferable example of the “3- to 10-membered non-aromatic heterocyclic group” includes aziridinyl, azetidinyl, pyrrolidinyl, piperidinyl, azepanyl, piperazinyl, diazepanyl, morpholinyl, thiomorpholinyl, 1,1-dioxothiomorpholinyl, tetrahydrofuryl, and tetrahydropyranyl.

The “4- to 10-membered non-aromatic heterocyclic group” represents

(1) a monocyclic or a bicyclic non-aromatic heterocyclic group
(2) having 4 to 10 atoms in the ring,
(3) containing 1 to 2 heteroatoms among the atoms of the ring,
(4) optionally containing 1 to 2 double bonds in the ring,
(5) optionally containing 1 to 3 carbonyl, sulfinyl, or sulfonyl in the ring.

If the group contains nitrogen in the ring, the nitrogen may have a bond not participating in the formation of the ring. The group includes, for specific example, azetidinyl, pyrrolidinyl, piperidinyl, azepanyl, azocanyl, piperazinyl, diazepanyl, diazocanyl, diazabicyclo[2.2.1]heptyl, morpholinyl, thiomorpholinyl, 1,1-dioxothiomorpholinyl, oxetanyl, tetrahydrofuryl, tetrahydropyranyl, dioxanyl, tetrahydrothienyl, tetrahydrothiopyranyl, oxazolidinyl, and thiazolidinyl.

The preferable example of the “4- to 10-membered non-aromatic heterocyclic group” includes azetidinyl, pyrrolidinyl, piperidinyl, azepanyl, piperazinyl, diazepanyl, morpholinyl, thiomorpholinyl, 1,1-dioxothiomorpholinyl, tetrahydrofuryl, and tetrahydropyranyl.

The “C₃₋₁₀ cycloalkyl-C₁₋₆ alkyl” represents a group obtained by substituting any one hydrogen of the above defined “C₁₋₆ alkyl” with the above defined “C₃₋₁₀ cycloalkyl”, and includes, for specific example, cyclopropylmethyl, cyclobutylmethyl, cyclopentylmethyl, cyclohexylmethyl, cycloheptylmethyl, cyclooctylmethyl, cyclononylmethyl, cyclodecylmethyl, bicyclo[2.2.1]heptylmethyl (norbornylmethyl), and bicyclo [4.4.0] decylmethyl (decarylmethyl).

The “C₆₋₁₀ aryl-C₁₋₆ alkyl” represents a group obtained by substituting any one hydrogen of the above defined “C₁₋₆ alkyl” with the above defined “C₆₋₁₀ aryl”, and includes, for specific example, benzyl, 1-naphthylmethyl, 2-naphthylmethyl, phenethyl, 1-naphthylethyl, and 2-naphthylethyl.

The “5- to 10-membered heteroaryl-C₁₋₆ alkyl” represents a group obtained by substituting any one hydrogen of the above defined “C₁₋₆ alkyl” with the above defined “5- to 10-membered heteroaryl”, and includes, for specific example, furylmethyl, thienylmethyl, pyrrolylmethyl, imidazolylmethyl, triazolylmethyl, tetrazolylmethyl, thiazolylmethyl, pyrazolylmethyl, oxazolylmethyl, isoxazolylmethyl, isothiazolylmethyl, furazanylmethyl, thiadiazolylmethyl, oxadiazolylmethyl, pyridylmethyl, pyrazinylmethyl, pyridazinylmethyl, pyrimidinylmethyl, triazinylmethyl, furylethyl, thienylethyl, pyrrolylethyl, imidazolylethyl, triazolylethyl, tetrazolylethyl, thiazolylethyl, pyrazolylethyl, oxazolylethyl, isoxazolylethyl, isothiazolylethyl, furazanylethyl, thiadiazolylethyl, oxadiazolylethyl, pyridylethyl, pyrazinylethyl, pyridazinylethyl, pyrimidinylethyl, and triazinylethyl.

The preferable example of the “5- to 10-membered heteroaryl C₁₋₆ alkyl” includes furylmethyl, thienylmethyl, pyrrolylmethyl, imidazolylmethyl, thiazolylmethyl, pyrazolylmethyl, oxazolylmethyl, isoxazolylmethyl, isothiazolylmethyl, pyridylmethyl, pyrimidinylmethyl, furylethyl, thienylethyl, pyrrolylethyl, imidazolylethyl, thiazolylethyl, pyrazolylethyl, oxazolylethyl, isoxazolylethyl, isothiazolylethyl, pyridylethyl, and pyrimidinylethyl.

The “3- to 10-membered non-aromatic heterocyclic-C₁₋₆ alkyl” represents a group obtained by substituting any one hydrogen of the above defined “C₁₋₆ alkyl” with the above defined “3- to 10-membered heterocyclic group”, and includes, for specific example, aziridinylmethyl, azetidinylmethyl, pyrrolidinylmethyl, piperidinylmethyl, azepanylmethyl, azocanylmethyl, piperazinylmethyl, diazepanylmethyl, diazocanylmethyl, morpholinylmethyl, thiomorpholinylmethyl, 1,1-dioxothiomorpholinylmethyl, oxiranylmethyl, oxetanylmethyl, tetrahydrofurylmethyl, tetrahydropyranylmethyl, dioxanylmethyl, tetrahydrothienylmethyl, tetrahydrothiopyranylmethyl, oxazolidinylmethyl, thiazolidinylmethyl, aziridinylethyl, azetidinylethyl, pyrrolidinylethyl, piperidinylethyl, azepanylethyl, azocanylethyl, piperazinylethyl, diazepanylethyl, diazocanylethyl, morpholinylethyl, thiomorpholinylethyl, 1,1-dioxothiomorpholinylethyl, oxiranylethyl, oxetanylethyl, tetrahydrofurylethyl, tetrahydropyranylethyl, dioxanylethyl, tetrahydrothienylethyl, tetrahydrothiopyranylethyl, oxazolidinylethyl, and thiazolidinylethyl.

The preferable example of the “3- to 10-membered non-aromatic heterocyclic-C₁₋₆ alkyl” includes azetidinylmethyl, pyrrolidinylmethyl, piperidinylmethyl, azepanylmethyl, piperazinylmethyl, diazepanylmethyl, morpholinylmethyl, thiomorpholinylmethyl, tetrahydrofurylmethyl, azetidinylethyl, pyrrolidinylethyl, piperidinylethyl, azepanylethyl, piperazinylethyl, diazepanylethyl, morpholinylethyl, thiomorpholinylethyl, and tetrahydrofurylethyl.

The “C₁₋₆ alkoxy” represents a group obtained by adding oxygen to the terminal of the above defined “C₁₋₆ alkyl”, and includes, for specific example, methoxy, ethoxy, 1-propoxy (n-propoxy), 2-propoxy (i-propoxy), 2-methyl-1-propoxy (i-butoxy), 2-methyl-2-propoxy (t-butoxy), 1-butoxy (n-butoxy), 2-butoxy (s-butoxy), 1-pentyloxy, 2-pentyloxy, 3-pentyloxy, 2-methyl-1-butoxy, 3-methyl-1-butoxy, 2-methyl-2-butoxy, 3-methyl-2-butoxy, 2,2-dimethyl-1-propoxy, 1-hexyloxy, 2-hexyloxy, 3-hexyloxy, 2-methyl-1-pentyloxy, 3-methyl-1-pentyloxy, 4-methyl-1-pentyloxy, 2-methyl-2-pentyloxy, 3-methyl-2-pentyloxy, 4-methyl-2-pentyloxy, 2-methyl-3-pentyloxy, 3-methyl-3-pentyloxy, 2,3-dimethyl-1-butoxy, 3,3-dimethyl-1-butoxy, 2,2-dimethyl-1-butoxy, 2-ethyl-1-butoxy, 3,3-dimethyl-2-butoxy, and 2,3-dimethyl-2-butoxy.

The “C₁₋₆ alkylthio” represents a group obtained by adding sulfur to the terminal of the above defined “C₁₋₆ alkyl”, and includes, for specific example, methylthio, ethylthio, 1-propylthio (n-propylthio), 2-propylthio (i-propylthio), 2-methyl-1-propylthio (i-butylthio), 2-methyl-2-propylthio (t-butylthio), 1-butylthio (n-butylthio), 2-butylthio (s-butylthio), 1-pentylthio, 2-pentylthio, 3-pentylthio, 2-methyl-1-butylthio, 3-methyl-1-butylthio, 2-methyl-2-butylthio, 3-methyl-2-butylthio, 2,2-dimethyl-1-propylthio, 1-hexylthio, 2-hexylthio, 3-hexylthio, 2-methyl-1-pentylthio, 3-methyl-1-pentylthio, 4-methyl-1-pentylthio, 2-methyl-2-pentylthio, 3-methyl-2-pentylthio, 4-methyl-2-pentylthio, 2-methyl-3-pentylthio, 3-methyl-3-pentylthio, 2,3-dimethyl-1-butylthio, 3,3-dimethyl-1-butylthio, 2,2-dimethyl-1-butylthio, 2-ethyl-1-butylthio, 3,3-dimethyl-2-butylthio, and 2,3-dimethyl-2-butylthio.

The “C₃₋₆ alkenyloxy” represents a group obtained by adding oxygen to the terminal of the above defined “C₃₋₆ alkenyl”, and includes, for specific example, 2-propenyloxy (allyloxy), 2-butenyloxy, 3-butenyloxy, pentenyloxy, and hexenyloxy.

The “C₃₋₆ alkenylthio” represents a group obtained by adding sulfur to the terminal of the above defined “C₃₋₆ alkenyl”, and includes, for specific example, 2-propenylthio (allylthio), 2-butenylthio, 3-butenylthio, pentenylthio, and hexenylthio.

The “C₃₋₆ alkynyloxy” represents a group obtained by adding oxygen to the terminal of the above defined “C₃₋₆ alkynyl”, and includes, for specific example, 2-propynyloxy, 2-butynyloxy, 3-butynyloxy, pentynyloxy, and hexynyloxy.

The “C₃₋₆ alkynylthio” represents a group obtained by adding sulfur to the terminal of the above defined “C₃₋₆ alkynyl”, and includes, for specific example, 2-propynylthio, 2-butynylthio, 3-butynylthio, pentynylthio, and hexynylthio.

The “C₃₋₁₀ cycloalkoxy” represents a group obtained by adding oxygen to the terminal of the above defined “C₃₋₁₀ cycloalkyl”, and includes, for specific example, cyclopropoxy, cyclobutoxy, cyclopentyloxy, cyclohexyloxy, cycloheptyloxy, and cyclooctyloxy.

The “C₃₋₁₀ cycloalkylthio” represents a group obtained by adding sulfur to the terminal of the above defined “C₃₋₁₀ cycloalkyl”, and includes, for specific example, cyclopropylthio, cyclobutylthio, cyclopentylthio, cyclohexylthio, cycloheptylthio, and cyclooctylthio.

The “C₆₋₁₀ aryloxy” represents a group obtained by adding oxygen to the terminal of the above defined “C₆₋₁₀ aryl”, and includes, for specific example, phenoxy, 1-naphthoxy, 2-naphthoxy, indenyloxy, azulenyloxy, and heptalenyloxy.

The “C₆₋₁₀ arylthio” represents a group obtained by adding sulfur to the terminal of the above defined “C₆₋₁₀ aryl”, and includes, for specific example, phenylthio, 1-naphthylthio, 2-naphthylthio, indenylthio, azulenylthio, and heptalenylthio.

The “5- to 10-membered heteroaryloxy” represents a group obtained by adding oxygen to the terminal of the above defined “5- to 10-membered heteroaryl”, and includes, for specific example, furyloxy, thienyloxy, pyrrolyloxy, imidazolyloxy, triazolyloxy, thiazolyloxy, pyrazolyloxy, oxazolyloxy, isoxazolyloxy, isothiazolyloxy, furazanyloxy, thiadiazolyloxy, oxadiazolyloxy, pyridyloxy, pyrazinyloxy, pyridazinyloxy, pyrimidinyloxy, and triazinyloxy.

The “5- to 10-membered heteroarylthio” represents a group obtained by adding sulfur to the terminal of the above defined “5- to 10-membered heteroaryl”, and includes, for specific example, furylthio, thienylthio, pyrrolylthio, imidazolylthio, triazolylthio, thiazolylthio, pyrazolylthio, oxazolylthio, isoxazolylthio, isothiazolylthio, furazanylthio, thiadiazolylthio, oxadiazolylthio, pyridylthio, pyrazinylthio, pyridazinylthio, pyrimidinylthio, and triazinylthio.

The “4- to 10-membered non-aromatic heterocyclicoxy group” represents a group obtained by adding oxygen to the terminal of the above defined “4- to 10-membered non-aromatic heterocyclic group”, and includes, for specific example, azetidinyloxy, pyrrolidinyloxy, piperidinyloxy, azepanyloxy, azoeanyloxy, piperazinyloxy, diazepanyloxy, diazocanyloxy, morpholinyloxy, thiomorpholinyloxy, 1,1-dioxothiomorpholinyloxy, oxetanyloxy, tetrahydrofuryloxy, tetrahydropyranyloxy, tetrahydrothienyloxy, and tetrahydrothiopyranyloxy.

The “4- to 10-membered non-aromatic heterocyclicthio group” represents a group obtained by adding sulfur to the terminal of the above defined “4- to 10-membered non-aromatic heterocyclic group”, and includes, for specific example, azetidinylthio, pyrrolidinylthio, piperidinylthio, azepanylthio, azocanylthio, piperazinylthio, diazepanylthio, diazocanylthio, oxetanylthio, tetrahydrofurylthio, tetrahydropyranylthio, tetrahydrothienylthio, and tetrahydrothiopyranylthio.

The “mono-C₁₋₆ alkylamino” represents a group obtained by substituting one hydrogen of amino with the above defined “C₁₋₆ alkyl”, and includes, for specific example, methylamino, ethylamino, 1-propylamino (n-propylamino), 2-propylamino (i-propylamino), 2-methyl-1-propylamino (1-butylamino), 2-methyl-2-propylamino (t-butylamino), 1-butylamino (n-butylamino), 2-butylamino (s-butylamino), 1-pentylamino, 2-pentylamino, 3-pentylamino, 2-methyl-1-butylamino, 3-methyl-1-butylamino, 2-methyl-2-butylamino, 3-methyl-2-butylamino, 2,2-dimethyl-1-propylamino, 1-hexylamino, 2-hexylamino, 3-hexylamino, 2-methyl-1-pentylamino, 3-methyl-1-pentylamino, 4-methyl-1-pentylamino, 2-methyl-2-pentylamino, 3-methyl-2-pentylamino, 4-methyl-2-pentylamino, 2-methyl-3-pentylamino, 3-methyl-3-pentylamino, 2,3-dimethyl-1-butylamino, 3,3-dimethyl-1-butylamino, 2,2-dimethyl-1-butylamino, 2-ethyl-1-butylamino, 3,3-dimethyl-2-butylamino, and 2,3-dimethyl-2-butylamino.

The “mono-C₃₋₁₀ cycloalkylamino” represents a group obtained by substituting one hydrogen of amino with the above defined “C₃₋₁₀ cycloalkyl”, and includes, for specific example, cyclopropylamino, cyclobutylamino, cyclopentylamino, cyclohexylamino, cycloheptylamino, and cyclooctylamino.

The “mono-C₆₋₁₀ arylamino” represents a group obtained by substituting one hydrogen of amino with the above defined “C₆₋₁₀ aryl”, and includes, for specific example, phenylamino, 1-naphthylamino, 2-naphthylamino, indenylamino, azulenylamino, and heptalenylamino.

The “mono-5- to 10-membered heteroarylamino” represents a group obtained by substituting one hydrogen of amino with the above defined “5- to 10-membered heteroaryl”, and includes, for specific example, furylamino, thienylamino, pyrrolylamino, imidazolylamino, triazolylamino, tetrazolylamino, thiazolylamino, pyrazolylamino, oxazolylamino, isoxazolylamino, isothiazolylamino, furazanylamino, thiadiazolylamino, oxadiazolylamino, pyridylamino, pyrazinylamino, pyridazinylamino, pyrimidinylamino, and triazinylamino.

The preferable example of the “mono-5- to 10-membered heteroarylamino” includes furylamino, thienylamino, pyrrolylamino, imidazolylamino, thiazolylamino, pyrazolylamino, oxazolylamino, isoxazolylamino, isothiazolylamino, pyridylamino, and pyrimidinylamino.

The “mono-4- to 10-membered non-aromatic heterocyclic amino” represents a group obtained by substituting one hydrogen of amino with the above defined “4- to 10-membered non-aromatic heterocyclic group”, and includes, for specific example, azetidinylamino, pyrrolidinylamino, piperidinylamino, azepanylamino, azocanylamino, piperazinylamino, diazepanylamino, diazocanylamino, morpholinylamino, thiomorpholinylamino, 1,1-dioxothiomorpholinylamino, oxetanylamino, tetrahydrofurylamino, tetrahydropyranylamino, tetrahydrothienylamino, and tetrahydrothiopyranylamino.

The preferable example of the “mono-4- to 10-membered non-aromatic heterocyclic amino” includes pyrrolidinylamino, piperidinylamino, azepanylamino, piperazinylamino, diazepanylamino, morpholinylamino, thiomorpholinylamino, and tetrahydrofurylamino.

The “di-C₁₋₆ alkylamino” represents a group obtained by substituting two hydrogen of amino with the same or different groups of the above defined “C₁₋₆ alkyl”, and includes, for specific example, N,N-dimethylamino, N,N-diethylamino, N,N-di-n-propylamino, N,N-di-1-propylamino, N,N-di-n-butylamino, N,N-di-1-butylamino, N,N-di-s-butylamino, N,N-di-t-butylamino, N-ethyl-N-methylamino, N-n-propyl-N-methylamino, N-1-propyl-N-methylamino, N-n-butyl-N-methylamino, N-1-butyl-N-methylamino, N-s-butyl-N-methylamino, and N-t-butyl-N-methylamino.

Each of the substituents in the compound of the present invention represented by the above formula (I) will be described below.

(Meaning of R¹)

R¹ represents a 3- to 10-membered non-aromatic heterocyclic group wherein the group is limited to a group having nitrogen as a ring constituent atom and the nitrogen having a bonding hand, or a group represented by the formula —NR^{11a}R^{11b}, wherein R^{11a} and Rub may be the same or different and each represents hydrogen, C₁₋₆ alkyl, C₃₋₆ alkenyl, C₃₋₆ alkynyl, C₃₋₁₀ cycloalkyl, C₆₋₁₀ aryl, 5- to 10-membered heteroaryl or a 4- to 10-membered non-aromatic heterocyclic group, and R^{11a} and R^{11b} may be substituted with a substituent selected from Substituent Group A or Substituent Group B.

R¹ may be substituted with a substituent selected from Substituent Group A or Substituent Group B.

The preferable example of R¹ includes a group represented by the formula (II):

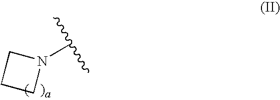

wherein a represents an integer of 1 to 4;
a group represented by the formula (III):

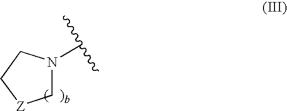

wherein b represents an integer of 1 to 3, and Z represents oxygen, sulfur, carbonyl, sulfonyl, or a group represented by the formula —NR^{Z}—, wherein R^{Z} represents hydrogen or C₁₋₆ alkyl, and the groups represented by the formula (II) or (III) may be substituted with a substituent selected from Substituent Group A or Substituent Group B; or
a group represented by the formula —NR^{11c}R^{11d}, wherein R^{11c} represents hydrogen or C₁₋₆ alkyl, and R^{11d} represents C₁₋₆ alkyl or a group represented by the formula (IV):

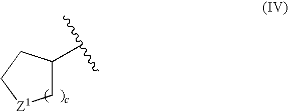

wherein c represents an integer of 1 to 3, and Z¹ represents oxygen, sulfur, carbonyl, sulfonyl or a group represented by the formula —NR^{Z1}—, wherein R^{Z1} represents hydrogen or C₁₋₆ alkyl, and R^{11d} may be substituted with a substituent selected from Substituent Group A or Substituent Group B.

The more preferable example of R¹ includes azetidin-1-yl, pyrrolidin-1-yl, piperidin-1-yl, azepan-1-yl, piperazin-1-yl, diazepan-1-yl, morpholin-4-yl, thiomorpholin-4-yl, 1,1-dioxothiomorpholin-4-yl, or a group represented by the formula —NR^{11e}R^{11f}, wherein R^{11e} represents hydrogen or C₁₋₆ alkyl, R^{11f} represents C₁₋₆ alkyl, pyrrolidin-3-yl, piperidin-3-yl, piperidin-4-yl or tetrahydropyran-4-yl, and R^{11f} may be substituted with a substituent selected from Substituent Group D, and each of the above substituents may be substituted with a substituent selected from Substituent Group D.

The even more preferable example of R¹ includes azetidin-1-yl, pyrrolidin-1-yl, piperidin-1-yl, piperazin-1-yl, diazepan-1-yl, morpholin-4-yl, and each of the above substituents may be substituted with a substituent selected from Substituent Group E, or a group represented by the formula —NR^{11g}R^{11h}, wherein R^{11g} represents hydrogen or methyl, R^{11h} represents n-propyl, n-butyl, pyrrolidin-3-yl, piperidin-3-yl, piperidin-4-yl or tetrahydropyran-4-yl, and R^{11h} may be substituted with a substituent selected from Substituent Group F.

The especially preferable example of R¹ includes azetidin-1-yl, pyrrolidin-1-yl, piperidin-1-yl or piperazin-1-yl, wherein azetidin-1-yl may be substituted with a substituent selected from Substituent Group G and pyrrolidin-1-yl, piperidin-1-yl and piperazin-1-yl are substituted with a substituent selected from Substituent Group G, or a group represented by the formula —N(CH₃)R¹¹ⁱ Ii wherein R¹¹ⁱ represents n-propyl, n-butyl, pyrrolidin-3-yl or piperidin-4-yl, and R¹¹ⁱ is substituted with a substituent selected from Substituent Group H.

The most preferable example of R¹ includes azetidin-1-yl, pyrrolidin-1-yl, piperidin-1-yl or piperazin-1-yl, wherein azetidin-1-yl may be substituted with a substituent selected from Substituent Group G-1 and pyrrolidin-1-yl, piperidin-1-yl and piperazin-1-yl are substituted with a substituent selected from Substituent Group G-1, or azetidin-1-yl having dimethylamino, pyrrolidin-1-yl having dimethylamino or piperidin-1-yl having dimethylamino, a group represented by the formula —N(CH₃)R^{11j} wherein R^{11j} represents 1-methylpiperidin-4-yl or 1-ethylpiperidin-4-yl, azetidin-1-yl optionally substituted with a substituent selected from Substituent Group G-2, pyrrolidin-1-yl substituted with a substituent selected from Substituent Group G-2, piperidin-1-yl substituted with a substituent selected from Substituent Group G-2 or a group represented by the formula —N(CH₃)R^{11k}, wherein R^{11k} represents 3-(dimethylamino)propyl or 1-[2-(dimethylamino)ethyl]piperidin-4-yl.

The most preferable example of R¹ also includes [2-(dimethylamino)ethyl]piperazin-1-yl, 4-pyrrolidin-1-ylpiperidin-1-yl, 4-[(dimethylamino)methyl]piperidin-1-yl, 4-azetidin-1-ylpiperidin-1-yl, 4-[3-(dimethylamino)azetidin-1-yl]piperidin-1-yl, 4-(4-methylpiperazin-1-yl)piperidin-1-yl, 4-(1-methylpiperidin-4-yl)piperazin-1-yl, 4-(1-methylazetidin-3-yl)piperazin-1-yl, 4-(dimethylamino)piperidin-1-yl, 4-(azetidin-1-ylmethyl)piperidin-1-yl, 4-(pyrrolidin-1-ylmethyl)piperidin-1-yl, (3S)-3-(dimethylamino)pyrrolidin-1-yl, (3R)-3-(dimethylamino)pyrrolidin-1-yl, azetidin-1-yl, pyrrolidin-1-yl, morpholin-4-yl, 4-methylpiperazin-1-yl, 3-hydroxyazetidin-1-yl, 1,3′-biazetidin-1′-yl, 3-(hydroxymethyl)azetidin-1-yl, 3-(dimethylamino)azetidin-1-yl, 3-[(dimethylamino)methyl] azetidin-1-yl, 4-hydroxypiperidin-1-yl, 4-(hydroxymethyl)piperidin-1-yl, (3R)-3-hydroxypyrrolidin-1-yl, (3S)-3-hydroxypyrrolidin-1-yl, 3-(azetidin-1-ylmethyl)azetidin-1-yl, 3-(2-dimethylaminoacetoxy)azetidin-1-yl, methyl(1-methylpiperidin-4-yl)amino, (1-ethylpiperidin-4-yl)(methyl)amino, [3-(dimethylamino)propyl](methyl)amino or {1-[2-(dimethylamino) ethyl]piperidin-4-yl}(methyl)amino.

(Meaning of Substituent Group a)

The Substituent Group A represents a group consisting of halogen, hydroxyl, mercapto, nitro, cyano and oxo.

(Meaning of Substituent Group B)

The Substituent Group B represents a group consisting of C₁₋₆ alkyl, C₂₋₆ alkenyl, C₂₋₆ alkynyl, C₃₋₁₀ cycloalkyl, C₆₋₁₀ aryl, 5- to 10-membered heteroaryl, a 3- to 10-membered non-aromatic heterocyclic group, C₁₋₆ alkoxy, C₃₋₆ alkenyloxy, C₃₋₆ alkynyloxy, C₃₋₁₀ cycloalkoxy, C₆₋₁₀ aryloxy, 5- to 10-membered heteroaryloxy, 4- to 10-membered non-aromatic heterocyclicoxy, C₁₋₆ alkylthio, C₃₋₆ alkenylthio, C₃₋₆ alkynylthio, C₃₋₁₀ cycloalkylthio, C₆- to arylthio, 5- to 10-membered heteroarylthio, 4- to 10-membered non-aromatic heterocyclicthio and a group represented by the formula -T¹-T²-T³, wherein T¹ represents a direct bond or C₁₋₆ alkylene, T² represents carbonyl, sulfinyl, sulfonyl, a group represented by the formula —C(═O)—O—, a group represented by the formula —O—C(═O)—, a group represented by the formula —SO₂—O—, a group represented by the formula —O—SO₂—, a group represented by the formula —NR^{T1}—, a group represented by the formula —C(═O)—NR^{T1}—, a group represented by the formula —NR^{T1}—C(═O)—, a group represented by the formula —SO₂—NR^{T1}— or a group represented by the formula —NR^{T1}—SO₂—, T³ represents hydrogen, C₁₋₆ alkyl, C₃₋₆ alkenyl, C₃₋₆ alkynyl, C₃₋₁₀ cycloalkyl, C₆₋₁₀ aryl, 5- to 10-membered heteroaryl or a 4- to 10-membered non-aromatic heterocyclic group, and R^{T1} represents hydrogen or C₁₋₆ alkyl.

Each group included in Substituent Group B may be substituted with a substituent selected from Substituent Group C.

(Meaning of Substituent Group C)

The Substituent Group C represents a group consisting of halogen, hydroxyl, mercapto, nitro, cyano, oxo, C₁₋₆ alkyl, C₂₋₆ alkenyl, C₂₋₆ alkynyl, C₃₋₁₀ cycloalkyl, C₆₋₁₀ aryl, 5- to 10-membered heteroaryl, a 3- to 10-membered non-aromatic heterocyclic group, C₁₋₆ alkoxy, C₁₋₆ alkylthio, mono-C₁₋₆ alkylamino and di-C₁₋₆ alkylamino.

(Meaning of Substituent Group D)

The Substituent Group D represents a group consisting of halogen, hydroxyl, mercapto, cyano, formyl, oxo, C₁₋₆ alkyl, C₃₋₁₀ cycloalkyl, C₁₋₆ alkoxy, amino, mono-C₁₋₆ alkylamino, di-C₁₋₆ alkylamino, azetidinyl, pyrrolidinyl, piperidinyl, piperazinyl, diazepanyl and a group represented by -T⁴-T⁵, wherein T⁴ represents carbonyl or sulfonyl, and T⁵ represents C₁₋₆ alkyl, C₃₋₁₀ cycloalkyl, azetidinyl, pyrrolidinyl, piperidinyl, hydroxyl, C₁₋₆ alkoxy, amino, mono-C₁₋₆ alkylamino or di-C₁₋₆ alkylamino.

Each group included in Substituent Group D may be substituted with hydroxyl, C₁₋₆ alkyl, di-C₁₋₆ alkylamino, azetidinyl or pyrrolidinyl.

(Meaning of Substituent Group E)

The Substituent Group E represents a group consisting of methyl, ethyl, dimethylamino, azetidinyl, pyrrolidinyl, piperidinyl and piperazinyl.

Each group included in Substituent Group E may be substituted with hydroxyl, methyl, dimethylamino, azetidinyl, pyrrolidinyl or piperidinyl.

(Meaning of Substituent Group F)

The Substituent Group F represents a group consisting of methyl, ethyl, n-propyl, acetyl, dimethylamino, diethylamino, azetidinyl, pyrrolidinyl and piperazinyl.

Each group included in Substituent Group F may be substituted with methyl or dimethylamino.

(Meaning of Substituent Group G)

The Substituent Group G represents a group consisting of dimethylamino, azetidinyl, pyrrolidinyl, piperidinyl, piperazinyl, dimethylaminomethyl, dimethylaminoethyl, azetidin-1-ylmethyl, pyrrolidin-1-ylmethyl and piperidin-1-ylmethyl.

Each group included in Substituent Group G may be substituted with methyl or dimethylamino.

(Meaning of Substituent Group G-1)

The Substituent Group G-1 represents a group consisting of azetidinyl, pyrrolidinyl, piperidinyl, piperazinyl, dimethylaminomethyl, dimethylaminoethyl, azetidin-1-ylmethyl, pyrrolidin-1-ylmethyl and piperidin-1-ylmethyl.

Each group included in Substituent Group G-1 may be substituted with methyl or dimethylamino.

(Meaning of Substituent Group G-2)

The Substituent Group G-2 represents a group consisting of hydroxyl, methoxy, hydroxymethyl and dimethylaminoacetoxy.

(Meaning of Substituent Group H)

The Substituent Group H represents a group consisting of dimethylamino, diethylamino, dimethylaminoethyl, dimethylaminopropyl and 1-methylazetidin-3-yl.

(Meaning of R² and R³)

R² and R³ represent hydrogen.

(Meaning of R⁴, R⁵, R⁶ and R⁷)

R⁴, R⁵, R⁶ and R⁷ may be the same or different and each represents hydrogen, halogen, hydroxyl, cyano, trifluoromethyl, C₁₋₆ alkyl, C₂₋₆ alkenyl, C₂₋₆ alkynyl, C₁₋₆ alkoxy, amino, mono-C₁₋₆ alkylamino, di-C₁₋₆ alkylamino or a group represented by the formula —CO—R¹², wherein R¹² represents hydrogen, hydroxyl, C₁₋₆ alkyl, C₁₋₆ alkoxy, amino, mono-C₁₋₆ alkylamino or di-C₁₋₆ alkylamino.

The preferable example of R⁴, R⁵, R⁶ and R⁷ includes hydrogen, halogen, C₁₋₆ alkyl, C₁₋₆ alkoxy and trifluoromethyl.

The more preferable example of R⁴, R⁵, R⁶ and R⁷ includes hydrogen, halogen and C₁₋₆ alkyl.

The even more preferable example of R⁴, R⁵, R⁶ and R⁷ includes hydrogen, fluorine, chlorine and methyl.

R⁴, R⁵, R⁶ and R⁷ may be in any one of the following cases: (1) all of them represent hydrogen, (2) all of them represent substituents other than hydrogen, and (3) some of them represent hydrogen and the others represent substituents other than hydrogen. Preferably, 2 to 4 of R⁴, R⁵, R⁶ and R⁷ represent hydrogen.

Preferable example for a group represented by the formula:

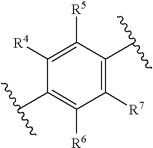

includes groups represented by the formulas:

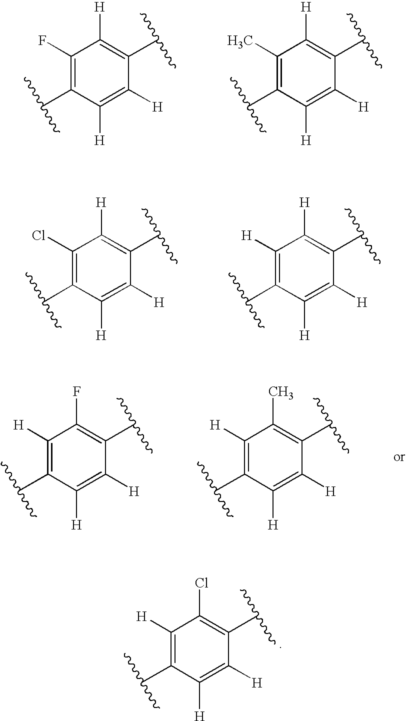

or a group represented by the formula:

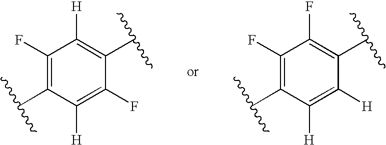

(Meaning of R⁸)

R⁸ represents hydrogen or C₁₋₆ alkyl.

The preferable example of R⁸ includes hydrogen.

(Meaning of R⁹)

R⁹ represents a 3- to 10-membered non-aromatic heterocyclic group wherein the group is limited to a group having nitrogen as a ring constituent atom and the nitrogen having a bonding hand, or a group represented by the formula —NR^{11a}R^{11b}, wherein R^{11a} and R^{11b} represent the same meaning as described above.

R⁹ may be substituted with a substituent selected from Substituent Group A or Substituent Group B.

The preferable example of R⁹ includes mono-C₁₋₆ alkylamino, mono-C₃₋₁₀ cycloalkylamino, mono-C₆₋₁₀ arylamino, mono-5- to 10-membered heteroarylamino or mono-4- to 10-membered non-aromatic heterocyclic amino, wherein R⁹ may be substituted with a substituent selected from Substituent Group A or Substituent Group B.

The more preferable example of R⁹ includes mono-C₃₋₁₀ cycloalkylamino or mono-C₆₋₁₀ arylamino, wherein R⁹ may be substituted with a substituent selected from Substituent Group A or Substituent Group B.

The even more preferable example of R⁹ includes mono-C₃₋₁₀ cycloalkylamino or mono-C₆₋₁₀ arylamino, wherein R⁹ may be substituted with a substituent selected from Substituent Group I.

The Substituent Group I represents a group consisting of halogen, trifluoromethyl, cyano, C₁₋₆ alkyl and C₁₋₆ alkoxy.

The especially preferable example of R⁹ includes cyclopentylamino, cyclohexylamino, cycloheptylamino and phenylamino, wherein R⁹ may be substituted with a substituent selected from Substituent Group I.

The most preferable example of R⁹ includes phenylamino optionally substituted with a substituent selected from the above Substituent Group I.

(Meaning of N)

n represents an integer of 1 or 2.

The preferable example of n includes 1.

(Meaning of X)

X represents a group represented by the formula —C(R¹⁰)═ or nitrogen, wherein R¹⁰ represents hydrogen, halogen, cyano, C₁₋₆ alkyl, C₂₋₆ alkenyl, C₂₋₆ alkynyl or a group represented by the formula —CO—R¹² wherein R¹² represents the same meaning as described above.

The preferable example of X includes a group represented by the formula —C(R^{10a})═ or nitrogen, wherein R^{10a} represents hydrogen, halogen or cyano.

The more preferable example of X includes a group represented by the formula —CH═ or nitrogen.

The preferable compound of the formula (I) includes a compound obtained by selecting respective aspects of R¹, R², R³, R⁴, R⁵, R⁶, R⁷, R⁸, R⁹, X and n in the compound and combining them arbitrarily.

The preferable compound of the formula (I) includes, other than the compounds described in Examples, the compounds illustrated below; but the present invention is not limited to the compounds described in Examples and the compounds illustrated below.
(1) N-(4-{[2-({[(1-ethylpiperidin-4-yl)(methyl)amino]carbonyl}amino)pyridin-4-yl]oxy}-2-fluorophenyl)-N′-(4-fluorophenyl)cyclopropane-1,1-dicarboxamide,
(2) N-(4-{[2-({[(1-ethylpiperidin-4-yl)(methyl)amino]carbonyl}amino)pyridin-4-yl]oxy}phenyl)-N′-(4-fluorophenyl)cyclopropane-1,1-dicarboxamide,
(3) N-{2-fluoro-4-[(2-{[(4-methyl-1,4-diazepan-1-yl)carbonyl]amino}pyridin-4-yl)oxy]phenyl}-N′-(4-fluorophenyl)cyclopropane-1,1-dicarboxamide,
(4) N-(4-fluorophenyl)-N′-{2-fluoro-4-[(2-{[(3-pyrrolidin-1-ylazetidin-1-yl)carbonyl]amino}pyridin-4-yl)oxy]phenyl}cyclopropane-1,1-dicarboxamide,
(5) N-{2-fluoro-4-[(2-{[(4-methylpiperazin-1-yl)carbonyl]amino}pyridin-4-yl)oxy]phenyl}-N′-(4-fluorophenyl)cyclopropane-1,1-dicarboxamide,
(6) N-[4-({2-[({4-[2-(dimethylamino)ethyl]-1,4-diazepan-1-yl}carbonyl)amino]pyridin-4-yl}oxy)-2-fluorophenyl]-N′-phenylcyclopropane-1,1-dicarboxamide,
(7) N-(4-{[2-({[3-(dimethylamino)azetidin-1-yl]carbonyl}amino)pyridin-4-yl]oxy}-2-fluorophenyl)-N′-(4-fluorophenyl)cyclopropane-1,1-dicarboxamide,
(8) N-(4-{[2-({[3-(dimethylamino)azetidin-1-yl]carbonyl}amino)pyridin-4-yl]oxy}phenyl)-N′-(4-fluorophenyl)cyclopropane-1,1-dicarboxamide,
(9) N-(4-{[2-({[3-(dimethylamino)azetidin-1-yl]carbonyl}amino)pyridin-4-yl]oxy}-2-fluorophenyl)-N′-phenylcyclopropane-1,1-dicarboxamide,
(10) N-[2-fluoro-4-({2-[({methyl[1-(1-methylazetidin-3-yl)piperidin-4-yl]amino}carbonyl)amino]pyridin-4-yl}oxy)phenyl]-N′-phenylcyclopropane-1,1-dicarboxamide,
(11) N-(2-fluoro-4-{[2-({[4-(1-methylazetidin-3-yl)piperazin-1-yl]carbonyl}amino)pyridin-4-yl]oxy}phenyl)-N′-phenylcyclopropane-1,1-dicarboxamide,
(12) N-(4-fluorophenyl)-N′-(4-{[2-({[4-(1-methylazetidin-3-yl)piperazin-1-yl]carbonyl}amino)pyridin-4-yl]oxy}phenyl)cyclopropane-1,1-dicarboxamide,
(13) N-(2-fluoro-4-{[2-({[(1-methylpiperidin-4-yl)amino]carbonyl}amino)pyridin-4-yl]oxy}phenyl)-N′-(4-fluorophenyl)cyclopropane-1,1-dicarboxamide,
(14) N-{2-fluoro-4-[(2-{[(4-hydroxy-1,4′-bipiperidin-1′-yl)carbonyl]amino}pyridin-4-yl)oxy]phenyl}-N′-phenylcyclopropane-1,1-dicarboxamide,
(15) N-(4-{[2-({[{1-[3-(dimethylamino)propyl]piperidin-4-yl}(methyl)amino]carbonyl}amino)pyridin-4-yl]oxy}-2-fluorophenyl)-N′-phenylcyclopropane-1,1-dicarboxamide,
(16) N-(4-{[2-({[(3-azetidin-1-ylpropyl)(methyl)amino]carbonyl}amino)pyridin-4-yl]oxy}-2-fluorophenyl)-N′-(4-fluorophenyl)cyclopropane-1,1-dicarboxamide,
(17) N-(2-fluoro-4-{[2-({[methyl(3-pyrrolidin-1-ylpropyl)amino]carbonyl}amino)pyridin-4-yl]oxy}phenyl)-N′-(4-fluorophenyl)cyclopropane-1,1-dicarboxamide,
(18) N-(4-{[2-({[[3-(dimethylamino)propyl] (methyl)amino]carbonyl}amino)pyridin-4-yl]oxy}-2-fluorophenyl)-N′-(4-fluorophenyl)cyclopropane-1,1-dicarboxamide,
(19) N-(2-fluoro-4-{[2-({[methyl(4-pyrrolidin-1-ylbutyl)amino]carbonyl}amino)pyridin-4-yl]oxy}phenyl)-N′-phenylcyclopropane-1,1-dicarboxamide,
(20) N-[2-fluoro-4-({2-[(morpholin-4-ylcarbonyl)amino]pyridin-4-yl}oxy)phenyl]-N′-(4-fluorophenyl)cyclopropane-1,1-dicarboxamide,
(21) N-[4-({2-[(azetidin-1-ylcarbonyl)amino]pyridin-4-yl}oxy)-2-fluorophenyl]-N′-(4-fluorophenyl)cyclopropane-1,1-dicarboxamide,
(22) N-(2-fluoro-4-{[2-({[methyl(3-morpholin-4-ylpropyl)amino]carbonyl}amino)pyridin-4-yl]oxy}phenyl)-N′-(4-fluorophenyl)cyclopropane-1,1-dicarboxamide,
(23) N-[2-fluoro-4-({2-[({methyl[3-(4-methylpiperazin-1-yl)propyl]amino}carbonyl)amino]pyridin-4-yl}oxy)phenyl]-N′-(4-fluorophenyl)cyclopropane-1,1-dicarboxamide,
(24) N-(4-fluorophenyl)-N′-[2-fluoro-4-({2-[(pyrrolidin-1-ylcarbonyl)amino]pyridin-4-yl}oxy)phenyl]cyclopropane-1,1-dicarboxamide,
(25) N-(2-fluoro-4-{[2-({[methyl(1-methylpiperidin-4-yl)amino]carbonyl}amino)pyridin-4-yl]oxy}phenyl)-N′-2-thienylcyclopropane-1,1-dicarboxamide,
(26) N-(2-fluoro-4-{[2-({[methyl(1-methylpiperidin-4-yl)amino]carbonyl}amino)pyridin-4-yl]oxy}phenyl)-N′-1,3-thiazol-2-ylcyclopropane-1,1-dicarboxamide,
(27) N-(2-fluoro-4-{[2-({[methyl(1-methylpiperidin-4-yl)amino]carbonyl}amino)pyridin-4-yl]oxy}phenyl)-N′-(5-methylisoxazol-3-yl)cyclopropane-1,1-dicarboxamide,
(28) N-(2-fluoro-4-{[2-({[methyl(1-methylpiperidin-4-yl)amino]carbonyl}amino)pyridin-4-yl]oxy}phenyl)-N′-(3-methylisoxazol-5-yl)cyclopropane-1,1-dicarboxamide,
(29) N-{2-fluoro-4-[(2-{[(4-hydroxypiperidin-1-yl)carbonyl]amino}pyridin-4-yl)oxy]phenyl}-N′-(4-fluorophenyl)cyclopropane-1,1-dicarboxamide,
(30) N-{2-fluoro-4-[(2-{[(4-methoxypiperidin-1-yl) carbonyl]amino}pyridin-4-yl)oxy]phenyl}-N′-(4-fluorophenyl)cyclopropane-1,1-dicarboxamide,
(31) N-{2-fluoro-4-[(2-{[(3-hydroxyazetidin-1-yl)carbonyl]amino}pyridin-4-yl)oxy]phenyl}-N′-(4-fluorophenyl)cyclopropane-1,1-dicarboxamide,
(32) N-{2-fluoro-4-[(2-{[(3-methoxyazetidin-1-yl)carbonyl]amino}pyridin-4-yl)oxy]phenyl}-N′-(4-fluorophenyl)cyclopropane-1,1-dicarboxamide,
(33) N-(2-fluoro-4-{[2-({[(2-methoxyethyl)(methyl)amino]carbonyl}amino)pyridin-4-yl]oxy}phenyl)-N′-(4-fluorophenyl)cyclopropane-1,1-dicarboxamide,
(34) N-(2-fluoro-4-{[2-({[4-(3-hydroxyazetidin-1-yl)piperidin-1-yl]carbonyl}amino)pyridin-4-yl]oxy}phenyl)-N′-(4-fluorophenyl)cyclopropane-1,1-dicarboxamide,
(35) N-(2-fluoro-4-{[2-({[methyl(tetrahydro-2H-pyran-4-yl)amino]carbonyl}amino)pyridin-4-yl]oxy}phenyl)-N′-(4-fluorophenyl)cyclopropane-1,1-dicarboxamide,
(36) N-(2-fluoro-4-{[2-({[methyl(1-methylpiperidin-3-yl)amino]carbonyl}amino)pyridin-4-yl]oxy}phenyl)-N′-(4-fluorophenyl)cyclopropane-1,1-dicarboxamide,
(37) N-[4-({2-[({3-[(dimethylamino)methyl]piperidin-1-yl}carbonyl)amino]pyridin-4-yl}oxy)phenyl]-N′-(4-fluorophenyl)cyclopropane-1,1-dicarboxamide,
(38) N-[4-({2-[({3-[(dimethylamino)methyl]pyrrolidin-1-yl}carbonyl)amino]pyridin-4-yl}oxy)-2-fluorophenyl]-N′-(4-fluorophenyl)cyclopropane-1,1-dicarboxamide,
(39) N-(2-fluoro-4-{[2-({[methyl(1-methylpyrrolidin-3-yl)amino]carbonyl}amino)pyridin-4-yl]oxy}phenyl)-N′-(4-fluorophenyl)cyclopropane-1,1-dicarboxamide,
(40) N-{2-fluoro-4-[(2-{[(3-hydroxypyrrolidin-1-yl)carbonyl]amino}pyridin-4-yl)oxy]phenyl}-N′-(4-fluorophenyl)cyclopropane-1,1-dicarboxamide,
(41) N-{2-fluoro-4-[(2-{[(3-methoxypyrrolidin-1-yl)carbonyl]amino}pyridin-4-yl)oxy]phenyl}-N′-(4-fluorophenyl)cyclopropane-1,1-dicarboxamide,
(42) N-{4-[(2-{[(3,4-dihydroxypyrrolidin-1-yl)carbonyl]amino}pyridin-4-yl)oxy]-2-fluorophenyl}-N′-(4-fluorophenyl)cyclopropane-1,1-dicarboxamide,
(43) N-{2-fluoro-4-[(2-{[(3-hydroxy-4-methoxypyrrolidin-1-yl)carbonyl]amino}pyridin-4-yl)oxy]phenyl}-N′-(4-fluorophenyl)cyclopropane-1,1-dicarboxamide,
(44) N-{4-[(2-1{[(3,4-dimethoxypyrrolidin-1-yl)carbonyl]amino}pyridin-4-yl)oxy]-2-fluorophenyl}-N′-(4-fluorophenyl)cyclopropane-1,1-dicarboxamide,
(45) N-{2-fluoro-4-[(2-1{[(3-hydroxypiperidin-1-yl)carbonyl]amino}pyridin-4-yl)oxy]phenyl}-N′-(4-fluorophenyl)cyclopropane-1,1-dicarboxamide,
(46) N-{2-fluoro-4-[(2-{[(3-methoxypiperidin-1-yl)carbonyl]amino}pyridin-4-yl)oxy]phenyl}-N′-(4-fluorophenyl)cyclopropane-1,1-dicarboxamide,
(47) N-(4-{[2-({[3-(dimethylamino)piperidin-1-yl]carbonyl}amino)pyridin-4-yl]oxy}-2-fluorophenyl)-N′-(4-fluorophenyl)cyclopropane-1,1-dicarboxamide

The more preferable compound of the formula (I) includes the compounds illustrated below;
(1) N-[4-({2-[({4-[2-(Dimethylamino)ethyl]piperazin-1-yl}carbonyl)amino]pyridin-4-yl}oxy)-2-fluorophenyl]-N′-(4-fluorophenyl)cyclopropane-1,1-dicarboxamide,
(2) N-(2-Fluoro-4-{[2-({[methyl(1-methylpiperidin-4-yl)amino]carbonyl}amino)pyridin-4-yl]oxy}phenyl)-N′-(4-fluorophenyl)cyclopropane-1,1-dicarboxamide,
(3) N-(4-Fluorophenyl)-N′-{2-fluoro-4-[(2-{[(4-pyrrolidin-1-ylpiperidin-1-yl)carbonyl]amino}pyridin-4-yl)oxy]phenyl}cyclopropane-1,1-dicarboxamide,
(4) N-[4-({2-[({4-[(Dimethylamino)methyl]piperidin-1-yl}carbonyl)amino]pyridin-4-yl}oxy)-2-fluorophenyl]-N′-(4-fluorophenyl)cyclopropane-1,1-dicarboxamide,
(5) N-{4-[(2-{[(4-Azetidin-1-ylpiperidin-1-yl)carbonyl]amino}pyridin-4-yl)oxy]-2-fluorophenyl}-N′-(4-fluorophenyl)cyclopropane-1,1-dicarboxamide,
(6) N-[4-({2-[({4-[3-(Dimethylamino)azetidin-1-yl]piperidin-1-yl}carbonyl)amino]pyridin-4-yl}oxy)-2-fluorophenyl]-N′-(4-fluorophenyl)cyclopropane-1,1-dicarboxamide,
(7) N-(2-Fluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide,
(8) N-(2-Fluoro-4-{[2-({[4-(1-methylpiperidin-4-yl)piperazin-1-yl]carbonyl}amino)pyridin-4-yl]oxy}phenyl)-N′-(4-fluorophenyl)cyclopropane-1,1-dicarboxamide,
(9) N-(2-Fluoro-4-{[2-({[4-(1-methylazetidin-3-yl)piperazin-1-yl]carbonyl}amino)pyridin-4-yl]oxy}phenyl)-N′-(4-fluorophenyl)cyclopropane-1,1-dicarboxamide,
(10) N-(4-{[2-({[4-(Dimethylamino)piperidin-1-yl]carbonyl}amino)pyridin-4-yl]oxy}-2-fluorophenyl)-N′-(4-fluorophenyl)cyclopropane-1,1-dicarboxamide,
(11) N-(4-{[2-({[4-(Azetidin-1-ylmethyl)piperidin-1-yl]carbonyl}amino)pyridin-4-yl]oxy}-2-fluorophenyl)-N′-(4-fluorophenyl)cyclopropane-1,1-dicarboxamide,
(12) N-(4-Fluorophenyl)-N′-(2-fluoro-4-{[2-({[4-(pyrrolidin-1-ylmethyl)piperidin-1-yl]carbonyl}amino)pyridin-4-yl]oxy}phenyl)cyclopropane-1,1-dicarboxamide,
(13) N-(4-{[2-({[(3S)-3-(Dimethylamino)pyrrolidin-1-yl]carbonyl}amino)pyridin-4-yl]oxy}-2-fluorophenyl)-N′-(4-fluorophenyl)cyclopropane-1,1-dicarboxamide,
(14) N-(4-{[2-({[(3R)-3-(Dimethylamino)pyrrolidin-1-yl]carbonyl}amino)pyridin-4-yl]oxy}-2-fluorophenyl)-N′-(4-fluorophenyl)cyclopropane-1,1-dicarboxamide,
(15) N-(2-Fluoro-4-{[2-({[methyl(1-methylpiperidin-4-yl)amino]carbonyl}amino)pyridin-4-yl]oxy}phenyl)-N′-phenylcyclopropane-1,1-dicarboxamide,
(16) N-(2-Fluoro-4-{[2-({[4-(4-methylpiperazin-1-yl)piperidin-1-yl]carbonyl}amino)pyridin-4-yl]oxy}phenyl)-N′-phenylcyclopropane-1,1-dicarboxamide,
(17) N-[4-({2-[({4-[3-(Dimethylamino)azetidin-1-yl]piperidin-1-yl}carbonyl)amino]pyridin-4-yl}oxy)-2-fluorophenyl]-N′-phenylcyclopropane-1,1-dicarboxamide,
(18) N-(4-{[2-({[(1-Ethylpiperidin-4-yl)(methyl)amino]carbonyl}amino)pyridin-4-yl]oxy}-2-fluorophenyl)-N′-phenylcyclopropane-1,1-dicarboxamide,
(19) N-[4-({2-[(Azetidin-1-ylcarbonyl)amino]pyridin-4-yl}oxy)-2-fluorophenyl]-N′-(4-fluorophenyl)cyclopropane-1,1-dicarboxamide,
(20) N-(4-Fluorophenyl)-N′-[2-fluoro-4-({2-[(pyrrolidin-1-ylcarbonyl)amino]pyridin-4-yl}oxy)phenyl]cyclopropane-1,1-dicarboxamide,
(21) N-{2-Fluoro-4-[(2-{[(3-hydroxyazetidin-1-yl)carbonyl]amino}pyridin-4-yl)oxy]phenyl}-N′-(4-fluorophenyl)cyclopropane-1,1-dicarboxamide,
(22) N-[4-({2-[(1,3′-Biazetidin-1′-ylcarbonyl)amino]pyridin-4-yl}oxy)-2-fluorophenyl]-N′-(4-fluorophenyl)cyclopropane-1,1-dicarboxamide,
(23) N-(2-Fluoro-4-{[2-({[3-(hydroxymethyl)azetidin-1-yl]carbonyl}amino)pyridin-4-yl]oxy}phenyl)-N′-(4-fluorophenyl)cyclopropane-1,1-dicarboxamide,
(24) N-(4-{[2-({[3-(Dimethylamino)azetidin-1-yl]carbonyl}amino)pyridin-4-yl]oxy}-2-fluorophenyl)-N′-(4-fluorophenyl)cyclopropane-1,1-dicarboxamide,
(25) N-[4-({2-[({3-[(Dimethylamino)methyl] azetidin-1-yl}carbonyl)amino]pyridin-4-yl}oxy)-2-fluorophenyl]-N′-(4-fluorophenyl)cyclopropane-1,1-dicarboxamide,
(26) N-{2-Fluoro-4-[(2-{[(4-hydroxypiperidin-1-yl)carbonyl]amino}pyridin-4-yl)oxy]phenyl}-N′-(4-fluorophenyl)cyclopropane-1,1-dicarboxamide,
(27) N-(2-Fluoro-4-{[2-({[4-(hydroxymethyl)piperidin-1-yl]carbonyl}amino)pyridin-4-yl]oxy}phenyl)-N′-(4-fluorophenyl)cyclopropane-1,1-dicarboxamide,
(28) N-(2-Fluoro-4-{[2-({[(3R)-3-hydroxypyrrolidin-1-yl]carbonyl}amino)pyridin-4-yl]oxy}phenyl)-N′-(4-fluorophenyl)cyclopropane-1,1-dicarboxamide,
(29) N-(2-Fluoro-4-{[2-({[(3S)-3-hydroxypyrrolidin-1-yl]carbonyl}amino)pyridin-4-yl]oxy}phenyl)-N′-(4-fluorophenyl)cyclopropane-1,1-dicarboxamide,
(30) N-[4-({2-[(Azetidin-1-ylcarbonyl)amino]pyridin-4-yl}oxy)-2,5-difluorophenyl]-N′-(4-fluorophenyl)cyclopropane-1,1-dicarboxamide,
(31) N-{2,5-Difluoro-4-[(2-{[(3-hydroxyazetidin-1-yl)carbonyl]amino}pyridin-4-yl)oxy]phenyl}-N′-(4-fluorophenyl)cyclopropane-1,1-dicarboxamide,
(32) N-(2,5-Difluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide,
(33) N-[2,5-Difluoro-4-({2-[({3-[(dimethylamino)methyl] azetidin-1-yl}carbonyl)amino]pyridin-4-yl}oxy)phenyl]-N′-(4-fluorophenyl)cyclopropane-1,1-dicarboxamide,
(34) N-(2,5-Difluoro-4-{[2-({[methyl(1-methylpiperidin-4-yl)amino]carbonyl}amino)pyridin-4-yl]oxy}phenyl)-N′-(4-fluorophenyl)cyclopropane-1,1-dicarboxamide,
(35) N-{4-[(2-{[3-(Azetidin-1-ylmethyl)azetidin-1-ylcarbonyl]amino}pyridin-4-yl)oxy]-2,5-difluorophenyl}-N′-(4-fluorophenyl)cyclopropane-1,1-dicarboxamide,
(36) N-(2,5-Difluoro-4-{[2-({[3-(hydroxymethyl)azetidin-1-yl]carbonyl}amino)pyridin-4-yl]oxy}phenyl)-N′-(4-fluorophenyl)cyclopropane-1,1-dicarboxamide,
(37) N-{2,5-Difluoro-4-[(4-{[(3-hydroxyazetidin-1-yl)carbonyl]amino}pyrimidin-6-yl)oxy]phenyl}-N′-(4-fluorophenyl)cyclopropane-1,1-dicarboxamide,
(38) N-[4-({4-[({3-[(Dimethylamino)methyl] azetidin-1-yl}carbonyl)amino]pyrimidin-6-yl}oxy)-2,5-difluorophenyl]-N′-(4-fluorophenyl)cyclopropane-1,1-dicarboxamide,
(39) N-(2,5-Difluoro-4-{[4-({[3-(hydroxymethyl)azetidin-1-yl]carbonyl}amino)pyrimidin-6-yl]oxy}phenyl)-N′-(4-fluorophenyl)cyclopropane-1,1-dicarboxamide,
(40) N-(2,5-Difluoro-4-{[4-({[methyl(1-methylpiperidin-4-yl)amino]carbonyl}amino)pyrimidin-6-yl]oxy}phenyl)-N′-(4-fluorophenyl)cyclopropane-1,1-dicarboxamide,
(41) N-(2,5-Difluoro-4-{[4-({[4-(4-methylpiperazin-1-yl)piperidin-1-yl]carbonyl}amino)pyrimidin-6-yl]oxy}phenyl)-N′-(4-fluorophenyl)cyclopropane-1,1-dicarboxamide,
(42) N-(4-{[2-({[4-(Dimethylamino)piperidin-1-yl]carbonyl}amino)pyridin-4-yl]oxy}-2,5-difluorophenyl)-N′-(4-fluorophenyl)cyclopropane-1,1-dicarboxamide,
(43) N-{2,5-Difluoro-4-[(2-{[(4-methylpiperazin-1-yl)carbonyl]amino}pyridin-4-yl]oxy}phenyl)-N′-(4-fluorophenyl)cyclopropane-1,1-dicarboxamide,
(44) N-{2,5-Difluoro-4-[(2-{[(4-hydroxypiperidin-1-yl)carbonyl]amino}pyridin-4-yl)oxy]phenyl}-N′-(4-fluorophenyl)cyclopropane-1,1-dicarboxamide,
(45) N-{4-[(2-{[(4-Azetidin-1-ylpiperidin-1-yl)carbonyl]amino}pyridin-4-yl)oxy]oxy}-2,5-difluorophenyl}-N′-(4-fluorophenyl)cyclopropane-1,1-dicarboxamide,
(46) N-(2,5-Difluoro-4-{[2-({[3-(2-dimethylaminoacetoxy)azetidin-1-yl]carbonyl}amino)pyridin-4-yl]oxy}phenyl)-N′-(4-fluorophenyl)cyclopropane-1,1-dicarboxamide,
(47) N-(2,5-Difluoro-4-{[2-({[(3S)-3-hydroxypyrrolidin-1-yl]carbonyl}amino)pyridin-4-yl]oxy}phenyl)-N′-(4-fluorophenyl)cyclopropane-1,1-dicarboxamide,
(48) N-(2,5-Difluoro-4-{[2-({[(3R)-3-hydroxypyrrolidin-1-yl]carbonyl}amino)pyridin-4-yl]oxy}phenyl)-N′-(4-fluorophenyl)cyclopropane-1,1-dicarboxamide.

The still more preferable compound of the formula (I) includes the compounds illustrated below;
(1) N-(2-Fluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide
(2) N-[4-({2-[(Azetidin-1-ylcarbonyl)amino]pyridin-4-yl}oxy)-2-fluorophenyl]-N′-(4-fluorophenyl)cyclopropane-1,1-dicarboxamide
(3) N-{2,5-Difluoro-4-[(2-{[(3-hydroxyazetidin-1-yl)carbonyl]amino}pyridin-4-yl)oxy]phenyl}-N′-(4-fluorophenyl)cyclopropane-1,1-dicarboxamide
(4) N-(2,5-Difluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide
(5) N-(2,5-Difluoro-4-{[2-({[methyl(1-methylpiperidin-4-yl)amino]carbonyl}amino)pyridin-4-yl]oxy}phenyl)-N′-(4-fluorophenyl)cyclopropane-1,1-dicarboxamide
(6) N-(2,5-Difluoro-4-{[2-({[3-(hydroxymethyl)azetidin-1-yl]carbonyl}amino)pyridin-4-yl]oxy}phenyl)-N′-(4-fluorophenyl)cyclopropane-1,1-dicarboxamide.

The phrase “may be substituted with a substituent selected from Substituent Group” or “optionally substituted with a substituent selected from Substituent Group” means “may be substituted with 1 to 3 substituents selected arbitrarily from the substituents described in the Substituent Group.”

The compounds of the present invention can be produced based on the description of WO 2007/023768.

The pyridine or pyrimidine derivative is generally mixed with an appropriate additive and formulated to use as a kinase inhibitor. But the pyridine or pyrimidine derivative may be used alone without any additive.

The above additives include excipients, binders, lubricants, disintegrators, coloring agents, taste correctives, emulsifiers, surfactants, dissolving aids, suspending agents, isotonizing agents, buffering agents, antiseptics, antioxidants, stabilizers, absorption accelerators and the like. These also may be appropriately combined to use if desired.

The excipients include, for example, lactose, white soft sugar, glucose, corn starch, mannitol, sorbitol, starch, pregelatinized starch, dextrin, crystalline cellulose, soft silicic anhydride, aluminum silicate, calcium silicate, magnesium aluminometasilicate and calcium hydrogenphosphate.

The binders include, for example, polyvinyl alcohol, methylcellulose, ethylcellulose, gum arabic, tragacanth, gelatin, shellac, hydroxypropylmethylcellulose, hydroxypropylcellulose, carboxymethylcellulose sodium, polyvinylpyrrolidone and macrogol.

The lubricants includes magnesium stearate, calcium stearate, sodium stearyl fumarate, talc, polyethylene glycol and colloidal silica.

The disintegrators includes, for example, crystalline cellulose, agar, gelatin, calcium carbonate, sodium hydrogencarbonate, calcium citrate, dextrin, pectin, low-substituted hydroxypropylcellulose, carboxymethylcellulose, carboxymethylcellulose calcium, croscarmellose sodium, carboxymethyl starch and carboxymethyl starch sodium.

The coloring agents include, for example, those approved for addition to pharmaceuticals, such as iron sesquioxide, yellow iron sesquioxide, carmine, caramel, β-carotene, titanium oxide, talc, riboflavin sodium phosphate, yellow aluminum lake and the like.

The taste correctives include cocoa powder, menthol, aromatic powders, mentha oil, borneol, powdered cinnamon bark and the like.

The emulsifiers or surfactants include, for example, stearyl triethanolamine, sodium lauryl sulfate, lauryl aminopropionic acid, lecitin, glycerin monostearate, sucrose fatty acid esters and glycerin fatty acid esters.

The dissolving aids include, for example, polyethylene glycol, propylene glycol, benzyl benzoate, ethanol, cholesterol, triethanolamine, sodium carbonate, sodium citrate, polysorbate 80 and nicotinamide.

The suspending agents include, for example, hydrophilic polymers such as polyvinyl alcohol, polyvinylpyrrolidone, methylcellulose, hydroxymethylcellulose, hydroxyethylcellulose and hydroxypropylcellulose, in addition to the above surfactants.

The isotonizing agents include, for example, glucose, sodium chloride, mannitol and sorbitol.

The buffering agents include, for example, buffer solutions of phosphate, acetate, carbonate and citrate.

The antiseptics include, for example, methylparaben, propylparaben, chlorobutanol, benzyl alcohol, phenethyl alcohol, dehydroacetic acid and sorbic acid.

The antioxidants include, for example, sulfite, ascorbic acid and α-tocopherol.

The stabilizers include those commonly used in pharmaceuticals.

The absorption accelerators include those commonly used in pharmaceuticals.

The formulation may be in an oral form such as tablets, powders, granules, capsules, syrups, lozenges and inhalants; an external application form such as suppositories, ointment, eye salve, tape, eye drops, nose drops, ear drops, pap and lotion; and an injection.

An oral formulation may be formulated by combining appropriately the above additives, and may be coated on the surface if necessary.

An external application may be formulated by combining appropriately the above additives, particularly excipients, binders, taste correctives, emulsifiers, surfactants, dissolving aids, suspending agents, isotonizing agents, antiseptics, antioxidants, stabilizers and absorption accelerators.

An injection may be formulated by combining appropriately the above additives, particularly emulsifiers, surfactants, dissolving aids, suspending agents, isotonizing agents, buffering agents, antiseptics, antioxidants, stabilizers and absorption accelerators.

The dose of the pyridine or pyrimidine derivative for use as a kinase inhibitor according to the present invention varies depending on symptoms and age of the patients, but it will ordinary be 0.1 mg to 10 g (preferably 1 mg to 2 g) for an oral formulation, 0.01 mg to 10 g (preferably 0.1 mg to 2 g) for an external application, and 0.01 mg to 10 g (preferably 0.1 mg to 2 g) for an injection, which is administrated once or divided over two to four times a day.

### EXAMPLES

### Pharmacological Test Examples

WO 2007/023768 has confirmed that the compound of the present invention has inhibitory activity against hepatocyte growth factor receptor, anti-tumor activity, inhibitory activity against angiogenesis, and inhibitory activity against cancer metastasis. The inhibitory activity against other receptor tyrosine kinases of the compound of the present invention was evaluated based on the following methods.

Abbreviations and terms used in the following Pharmacological Test Examples are listed as follows:

### (Abbreviation List)

VEGFR (Vascular endothelial growth factor receptor)
DNA (Deoxyribonucleic acid)
PCR (Polymerase chain reaction)
FBS (Fetal bovine serum)
PBS (Phosphate buffered saline)
Tris (Tris(hydroxymethyl)aminomethane, Tris (buffer))
PMSF (Phenylmethylsulfonyl fluoride)

### NP-40 (Nonidet P-40)

### DTT (Dithiothreitol)

EGTA (O,O-Bis(2-aminoethyleneglycol)-N,N,N′,N′-tetraacetic acid)
SDS (Sodium dodecyl sulfate)
BSA (Bovine serum albumin)
Hepes (N-[2-hydroxyethyl]piperazine-N′-[2-ethanesulfonic acid],
Hepes (buffer))
ATP (Adenosine 5′-triphosphate)
EDTA (Ethylenediamine tetraacetic acid)

### HTRF (Homogenous Time-Resolved Fluorescence)

ELISA (Enzyme-linked immunosorbent assay)

### Pharmacological Text Example 1

### An Inhibitory Activity on VEGFR-2 Tyrosine Kinase Activity

1. Cloning of VEGFR-2 tyrosine kinase and preparation of recombinant baculovirus solution

Cytoplasmic domain of VEGFR-2 (Genbank Accession No. L04947) is a 1.7 kb DNA fragment starting from lysine 791 and contains a stop codon, as described by Tarman et al. (Oncogene, 6(9), 1677-1683, 1991). This DNA fragment was isolated from a human placental cDNA library (obtained from Clontech Laboratories, Inc.) with two primers (obtained from TaKaRa Ex Taq™ Kit, TaKaRa) using a PCR method. This DNA fragment was cloned into a baculovirus transplace vector (pFastBac™-HT (obtained from GIBCO BRL)), to obtain a recombinant construct. An insect cell (Spodoptera frugiperda 9 (Sf9)) was transfected with it, and a VEGFR-2 recombinant baculovirus solution was prepared (preparation of the recombinant baculovirus can be found in a standard textbook (Bac-to-Bac Baculovirus Expression System (GIBCO BRL)).

### 2. Expression and Purification of VEGFR-2 Tyrosine Kinase

To Sf9 cells (3×10⁸ cells) suspended in a SF-90011 culture medium containing 2% FBS (obtained from Invitrogen Corp.), the above mentioned recombinant VEGFR-2 baculovirus solution (4 ml) was added and incubated with shaking at 27° C. for 48 hours. The recombinant VEGFR-2 baculovirus-infected cell culture was centrifuged at 4° C. at 1000 rpm for 5 minutes, and the supernatant was removed. The precipitated infected cells were suspended in 80 ml of ice-cold PBS, the suspension was centrifuged at 4° C. at 1000 rpm for 5 minutes, and the supernatant was removed. The precipitated infected cells were suspended in 40 ml of ice-cold Lysis Buffer (50 mM Tris-HCl (pH 8.5), 5 mM 2-mercaptoethanol, 100 mM KCl, 1 mM PMSF, and 1% (v/v) NP-40). This suspension was centrifuged at 4° C. at 12,000 rpm for 30 min to obtain a supernatant.

This supernatant was added to a Ni-NTA agarose column (3 ml, obtained from Qiagen) which had been equilibrated with 30 ml of Buffer A (20 mM Tris-HCl (pH 8.5), 5 mM 2-mercaptoethanol, 500 mM KCl, 20 mM imidazole, and 10% (v/v) glycerol). This column was washed sequentially with 30 ml of Buffer A, 6 ml of Buffer B (20 mM Tris-HCl (pH 8.5), 5 mM 2-mercaptoethanol, 1M KCl, and 10% (v/v) glycerol), and 6 ml of Buffer A. Then, to this column, 6 ml of Buffer C (20 mM Tris-HCl (pH 8.5), 5 mM 2-mercaptoethanol, 100 mM KCl, 100 mM imidazole, and 10% (v/v) glycerol) was added to obtain an eluate. This eluate was poured into a dialysis membrane (obtained from Spectrum Laboratories) and dialyzed with 1 liter of dialysis buffer (20 mM Tris-HCl (pH 7.5), 10% (v/v) glycerol, 1 mM DTT, 0.1 mM Na₃VO₄, 0.1 mM EGTA) at 4° C. overnight and stored at −80° C. until use. After the dialysis, a part of the eluate was subjected to a SDS electrophoresis, and the recombinant protein (His 6-VEGFR-2, a cytoplasmic domain of VEGFR-2 fused with 6 histidine residues on its N-terminus) detected at about 100 kDa Mw in Coomassie Brilliant Blue staining was subjected to protein quantification using BSA (obtained from Sigma Co. Ltd.) as a standard.

3. Measurement of Inhibitory Activity on VEGFR-2 Tyrosine Kinase

In each well of a 96-well round bottom plate (obtained from NUNC, product number 163320), 10 μl of a solution for kinase reaction (200 mM Hepes (pH 7.4), 80 mM MgCl₂, 16 mM MnCl₂, 2 mM Na₃VO₄), 250 ng of biotin-conjugated poly (Glu 4: Tyr 1) (biotin-poly (GT) obtained from Nihon Schering K.K.) (6 μl of 1/15 diluted solution with distilled water), 15 ng of His6-VEGFR-2 (10 μl of a 1/240 diluted solution with a 0.4% BSA solution), and a test substance solution in dimethyl sulfoxide (4 μl of 1/100 dilution with 0.1% BSA) were added to make a total volume of 30 μl. To them, 10 μl of 4 μM ATP (obtained from Sigma Co. Ltd.) diluted with distilled water was added, and the resultant was incubated for 10 minutes at 30° C., followed by addition of 10 μl of 500 mM EDTA (pH 8.0) (obtained from Wako Pure Chemical Industries, Ltd.) to obtain a kinase reaction solution.

To detect tyrosine-phosphorylated biotin-poly (GT), the Homogenous Time-Resolved Fluorescence (HTRF) method was used (Analytical Biochemistry, 269, 94-104, 1999). That is, 33 μl of the above mentioned kinase reaction solution and 17 μl of a diluent (50 mM Hepes (pH 7.4), 20 mM MgCl₂, 4 mM MnCl₂, 0.5 mM Na₃VO₄, 0.1% BSA, 100 mM EDTA) were added to each well of a 96-well half area black plate (obtained from COSTAR, product number 3694). To each well, 7.5 ng (25 μl of a 1/250 diluted solution with 20 mM Hepes (pH 7.0), 0.5 M KF, 0.1% BSA) of europium cryptate-labeled anti-phophotyrosine antibody (Eu (K) —PY20, obtained from Nihon Schering K.K.) and 250 ng (25 μl of a 62.5-fold diluted solution with 20 mM Hepes (pH 7.0), 0.5 M KF, 0.1% BSA) of XL665-labelled streptavidin (XL665-SA, obtained from Nihon Schering K.K.) were added and the fluorescence intensity of the each well was immediately measured at 665 nm and 620 nm with excitation wavelength of 337 nm using Discovery HTRF Microplate Analyzer (manufactured by Packard). A tyrosine phosphorylation ratio of Biotin-poly (GT) was calculated using a delta F % value described in HTRF standard examination textbook from Nihon Schering K.K. That is, the ratio (%) of delta F % of each well with an addition of a test substance was determined, assuming the delta F % value of the well with His6-VEGFR-2 but no test substance as 100%, and the delta F % value of the well without a test substance and without His6-VEGFR-2 as 0%. Based on this ratio (%), the concentration of the test substance required to inhibit 50% of VEGFR-2 kinase activity (IC₅₀) was calculated and shown in Table 1.

### Pharmacological Test Example 2

### Inhibitory Activity on Activities of Tyrosine Kinases Other Than VEGFR-2

### 1. Preparation and Storage of Test Substance Solution

A test compound was dissolved in dimethyl sulfoxide to make a 10 mM solution, which was stored in a dark place at 4° C. until use. When a biological activity (kinase activity) was determined, the test substance solution was diluted with dimethyl sulfoxide so that it had a 100-fold concentration of the test substance solution, then the resultant was prepared by 25-fold diluting with the following assay buffer (the concentration of dimethyl sulfoxide was 4%).

2. Measurement of Tyrosine Kinase Activity

Protein tyrosine kinases (recombinant human kinases) used in the study were the following products from Carna Biosciences Inc. (Kobe, Japan).

VEGFR-1 (FLT 1) (product number: 08-189)
VEGFR-3 (FLT 4) (product number: 08-190)
RON (product number: 08-152)
RET (product number: 08-159)
KIT (product number: 08-156)

Kinase activity was measured in a mobility shift assay (MSA) method (J. Biomolecular Screening, 11, 359-368, 2006) using QuickScout Screening Assist™ (Carna Biosciences Inc., commercial kit), and enzyme-linked immunoassay (ELISA).

1) Measurement of inhibitory action on tyrosine kinase activity of VEGFR-1, VEGFR-3, RON, and RET (MSA)

5 μl of the test substance solution obtained by dissolving or suspending in an assay buffer (20 mM Hepes, 0.01% Triton X-100, 1 mM dithiothreitol, pH 7.4) (4× final concentration) or 5 μl of a solvent (4% dimethyl sulfoxide-assay buffer) was dispensed into a polypropylene 384-well plate (Greiner Bio-One, product number 781280). Then, 5 μl of QuickScout Screening Assist™ MSA in an ATP/substrate/metal solution was added. Additionally, 10 μl of the kinase solution diluted with the assay buffer was added to initiate a reaction. For a blank, only 10 μl of the assay buffer was added. The kinase concentration and reaction conditions were in accordance with the protocol of QuickScout Screening Assist™ MSA. Then, the reaction was terminated by addition of 60 μl of a termination buffer of the QuickScout Screening Assist™ MSA, and the amount of a substrate (S) and a phosphorylated substrate (P) in the reaction solution were determined using LabChip3000 (Caliper Life Science, Massachusetts, U.S.A.) in accordance with the protocol of QuickScout Screening Assist™ MSA. The amounts of the S and P were represented by respective separated peak height. By assuming averaged signal from the well with enzyme but no test substance as 0% inhibition, and averaged signal from the well with no enzyme nor test substance as 100% inhibition, and approximating plots of the test substance concentration and inhibitory ratio to a 4-parameterd logistic curve based on the signals of each wells with the test substance, the concentration of test substance which exhibits 50% inhibition ratio (IC₅₀) was calculated and shown in Table 1. It was on the condition that the signal was determined as P/(P+S).

2) Measurement of Inhibitory Action on KIT (ELISA)

10 μl of the test substance solution obtained by dissolving or suspending in the assay buffer (15 mM Tris, 0.01% Tween 20, 2 mM DTT, pH 7.5) (4× final concentration), or 10 μl of a solvent (4% dimethyl sulfoxide-assay buffer) was dispensed into a DELFIA Streptavidin-coated clear plate (PerkinElmer Inc., product number 4009-0010). Then, 10 μl of an ATP/substrate/metal solution of QuickScout Screening Assist™ ELISA was added. Additionally, 20 μl of the kinase solution diluted with the assay buffer was added to initiate a reaction. For a blank, only 20 μl of the assay buffer was added. The assay method was in accordance with the protocol of QuickScout Screening Assist™ ELISA. After the reaction, the absorbance at 450 nm in each well was measured using SpectraMax (Molecular Devices, California, U.S.A). By assuming averaged signal from the well containing enzyme but no test substance as 0% inhibition, and averaged signal from the well with no enzyme nor test substance as 100% inhibition, and approximating plots of the test substance concentration and inhibition rate to a 4-parameterd logistic curve based on the signals of each well with an addition of the test substance, the concentration of the test substance which exhibits 50% inhibition rate (IC₅₀) was calculated and the results are shown in Table 1.

TABLE 1

| | Test Substance | | | | | |
|---|---|---|---|---|---|---|
| | VEGFR- | VEGFR-1 | VEGFR- | Ron | RET | KIT |
| N-(2-Fluoro-4{[2-({[4-(4- | 0.24 | 0.088 | 0.13 | 0.017 | 0.13 | 0.10 |
| methylpiperazin-1- | | | | | | |
| yl)piperidin-1- | | | | | | |
| yl]carbonyl}amino)pyridin-4- | | | | | | |
| yl]oxy}phenyl)-N′-(4- | | | | | | |
| fluorophenyl)cyclopropane- | | | | | | |
| 1,1-dicarboxamide | | | | | | |
| N-[4-({2-[(Azetidin-1- | 0.059 | 0.071 | 0.036 | 0.026 | 0.099 | 0.38 |
| Ylcarbonyl)amino]pyridin-4- | | | | | | |
| yl}oxy)-2-fluorophenyl]-N′-(4- | | | | | | |
| fluorophenyl)cyclopropane-1,1- | | | | | | |
| dicarboxamide | | | | | | |
| N-{2,5-Difluoro-4-[(2-{[(3- | 0.0095 | 0.026 | 0.010 | 0.0046 | 0.044 | 0.37 |
| hydroxyazetidin-1- | | | | | | |
| yl)carbony]amino}pyridin-4- | | | | | | |
| yl)oxy]phenyl}-N′-(4- | | | | | | |
| fluorophenyl)cyclopropane-1,1- | | | | | | |
| dicarboxamide | | | | | | |
| N-(2,5-Difluoro-4-{[2-({[4- | 0.10 | 0.021 | 0.022 | 0.0024 | 0.038 | 0.35 |
| (4-methylpiperazin-1-yl)piperidin- | | | | | | |
| 1-yl]carbonyl}amino)pyridin-4- | | | | | | |
| yl]oxy}phenyl)-N′-(4- | | | | | | |
| fluorophenyl)cyclopropane-1,1- | | | | | | |
| dicarboxamide | | | | | | |
| N-(2,5-Difluoro-4-{[2- | 0.095 | 0.012 | 0.011 | 0.0018 | 0.036 | 0.56 |
| ({[methyl(1-methylpiperidin-4- | | | | | | |
| yl)amino]carbonyl}amino)pyridin- | | | | | | |
| 4-yl]oxy}phenyl)-N′-(4- | | | | | | |
| fluorophenyl)cyclopropane-1,1- | | | | | | |
| dicarboxamide | | | | | | |
| N-(2,5-Difluoro-4-{[2-({[3- | 0.010 | 0.014 | 0.0062 | 0.0026 | 0.028 | 0.14 |
| (hydroxymethyl)azetidin-1- | | | | | | |
| yl]carbonyl}amino)pyridin-4- | | | | | | |
| yl]oxy}phenyl)-N′-(4- | | | | | | |
| fluorophenyl)cyclopropane-1,1- | | | | | | |
| dicarboxamide | | | | | | |

### Pharmacological Test Example 3

### Growth Inhibitory Activity on Human Umbilical Vein Endothelial Cells Stimulated by VEGF

Human umbilical vein endothelial cells (HUVECs) were isolated according to the reported method (Shin-seikagaku jikken kouza “saibou baiyou gijutu (Cell culturing techniques)” p. 197-202) and incubated to confluence using an EGM-2 culture medium (obtained from Sanko Junyaku Co., Ltd.) in a 5% CO₂ incubator (37° C.). The HUVECs were collected by a trypsin-EDTA treatment and washed with a culture medium (2% fetal bovine serum-supplemented Human endothelial SFM basal medium, obtained from Invitrogen Corp.), followed by suspending them in the medium to count the cell number. By diluting it with the medium, a 2×10⁴ cells/ml cell suspension was prepared. The cell suspension was dispensed into a 96-well plate (obtained from FALCON) by 100 μl each, and incubated at 37° C. in a 5% CO₂ incubator overnight. After the incubation, 50 μl of the test substance diluted with the medium was added to each well, and 50 μL of 80 ng/ml human recombinant VEGF (obtained from R&D systems, Inc.) diluted with the medium was added and further incubated for 3 days in the 5% CO₂ incubator (37° C.). After the incubation, 20 μl of Cell Counting Kit-8 (obtained from DOJINDO Laboratories) was added to each well, which were incubated for about 2 hours in the 5% CO₂ incubator (37° C.). After the incubation, the absorbance of the each well was measured using a plate reader MTP-500 (Corona Electric Co., Ltd) with a measurement wavelength of 450 nm and a control wavelength of 660 nm. A ratio of absorbance (%) of each well with the test substance against the wells without the test substance was determined, and based on this ratio, the concentration of the test substance required to inhibit 50% of cell proliferation (IC₅₀) was determined and the results are shown in Table 2.

TABLE 2

| | HUVEC Growth |
|---|---|
| Test Substance | IC₅₀ (μM) |
| N-(2-Fluoro-4{[2-({[4-(4- | 0.082 |
| methylpiperazin-1-yl)piperidin-1- | |
| yl]carbonyl}amino)pyridin-4- | |
| yl]oxy}phenyl)-N′-(4- | |
| fluorophenyl)cyclopropane-1,1- | |
| dicarboxamide | |
| N-[4-({2-[(Azetidin-1- | 0.038 |
| ylcarbonyl)amino]pyridin-4-yl}oxy)-2- | |
| fluorophenyl]-N′-(4- | |
| fluorophenyl)cyclopropane-1,1- | |
| dicarboxamide | |
| N-{2,5-Difluoro-4-[(2-{[(3-hydroxyazetidin- | 0.015 |
| 1-yl)carbony]amino}pyridin-4- | |
| yl)oxy]phenyl}-N′-(4- | |
| fluorophenyl)cyclopropane-1,1- | |
| dicarboxamide | |
| N-(2,5-Difluoro-4-{[2-({[4-(4- | 0.020 |
| methylpiperazin-1-yl)piperidin-1- | |
| yl]carbonyl}amino)pyridin-4-yl]oxy}phenyl)- | |
| N′-(4-fluorophenyl)cyclopropane-1,1- | |
| dicarboxamide | |
| N-(2,5-Difluoro-4-{[2-({[methyl(1- | 0.021 |
| methylpiperidin-4- | |
| yl)amino]carbonyl}amino)pyridin-4- | |
| yl]oxy}phenyl)-N′-(4- | |
| fluorophenyl)cyclopropane-1,1- | |
| dicarboxamide | |
| N-(2,5-Difluoro-4-{[2-({[3- | 0.0055 |
| (hydroxymethyl)azetidin-1- | |
| yl]carbonyl}amino)pyridin-4-yl]oxy}phenyl)- | |
| N′-(4-fluorophenyl)cyclopropane-1,1- | |
| dicarboxamide | |

What is claimed is:

1. A method of treating a disease associated with activation of VEGFR-1, VEGFR-2, VEGFR-3, RON, RET and/or KIT, comprising administering to a patient in need thereof, a compound represented by the following formula, a salt thereof or a hydrate of the foregoing: 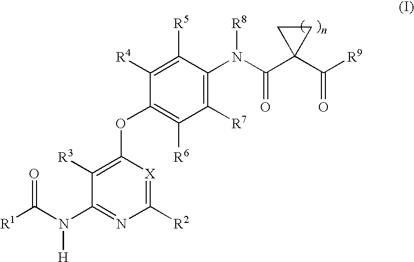
wherein R¹ represents a 3- to 10-membered non-aromatic heterocyclic group wherein the group is limited to a group having nitrogen as a ring constituent atom and the nitrogen having a bonding hand, or a group represented by the formula —NR^{11a}R^{11b}, wherein R^{11a} and R^{11b} may be the same or different and each represents hydrogen, C₁₋₆ alkyl, C₃₋₆ alkenyl, C₃₋₆ alkynyl, C₃₋₁₀ cycloalkyl, C₆₋₁₀ aryl, 5- to 10-membered heteroaryl or a 4- to 10-membered non-aromatic heterocyclic group, and R^{11a} and R^{11b} may be substituted with a substituent selected from Substituent Group A or Substituent Group B and R¹ may be substituted with a substituent selected from Substituent Group A or Substituent Group B;
R² and R³ represent hydrogen;
R⁴, K, R⁵, R⁶ and R⁷ may be the same or different and each represents hydrogen, halogen, hydroxyl, cyano, trifluoromethyl, C₁₋₆ alkyl, C₂₋₆ alkenyl, C₂₋₆ alkynyl, C₁₋₆ alkoxy, amino, mono-C₁₋₆ alkylamino, di-C₁₋₆ alkylamino or a group represented by the formula —CO—R¹², wherein R¹² represents hydrogen, hydroxyl, C₁₋₆ alkyl, C₁₋₆ alkoxy, amino, mono-C₁₋₆ alkylamino or di-C₁₋₆ alkylamino;
R⁸ represents hydrogen or C₁₋₆ alkyl;
R⁹ represents a 3- to 10-membered non-aromatic heterocyclic group wherein the group is limited to a group having nitrogen as a ring constituent atom and the nitrogen having a bonding hand, or a group represented by the formula —NR^{11a}R^{11b}, wherein R^{11a} and R^{11b} represent the same meaning as described above and R⁹ may be substituted with a substituent selected from Substituent Group A or Substituent Group B;
n represents an integer of 1 or 2; and
X represents a group represented by the formula —C(R¹⁰)═ or nitrogen, wherein R¹⁰ represents hydrogen, halogen, cyano, C₁₋₆ alkyl, C₂₋₆ alkenyl, C₂₋₆ alkynyl or a group represented by the formula —CO—R¹², wherein R¹² represents the same meaning as recited above;
wherein Substituent Group A consists of halogen, hydroxyl, mercapto, nitro, cyano and oxo;
wherein Substituent Group B consists of C₁₋₆ alkyl, C₂₋₆ alkenyl, C₂₋₆ alkynyl, C₃₋₁₀ cycloalkyl, C₆₋₁₀ aryl, 5- to 10-membered heteroaryl, a 3- to 10-membered non-aromatic heterocyclic group, C₁₋₆ alkoxy, C₃₋₆ alkenyloxy, C₃₋₆ alkynyloxy, C₃₋₁₀ cycloalkoxy, C₆₋₁₀ aryloxy, 5- to 10-membered heteroaryloxy, 4- to 10-membered non-aromatic heterocyclicoxy, C₁₋₆ alkylthio, C₃₋₆ alkenylthio, C₃₋₆ alkynylthio, C₃₋₁₀ cycloalkylthio, C₆₋₁₀ arylthio, 5- to 10-membered heteroarylthio, 4- to 10-membered non-aromatic heterocyclicthio and a group represented by the formula -T¹-T²-T³ and each group in Substituent Group B may be substituted with a substituent selected from Substituent Group C, wherein T¹ represents a direct bond or C₁₋₆ alkylene, T² represents carbonyl, sulfinyl, sulfonyl, a group represented by the formula —C(═O)—O—, a group represented by the formula —O—C(═O)—, a group represented by the formula —SO₂—O—, a group represented by the formula —O—SO₂—, a group represented by the formula —NR^{T1}—, a group represented by the formula —C(═O)—NR^{T1}—, a group represented by the formula —NR^{T1}—C(═O)—, a group represented by the formula —SO₂—NR^{T1}— or a group represented by the formula —NR^{T1}—SO₂—, T³ represents hydrogen, C₁₋₆ alkyl, C₃₋₆ alkenyl, C₃₋₆ alkynyl, C₃₋₁₀ cycloalkyl, C₆₋₁₀ aryl, 5- to 10-membered heteroaryl or a 4- to 10-membered non-aromatic heterocyclic group, and R^{T1} represents hydrogen or C₁₋₆ alkyl; and
wherein Substituent Group C consists of halogen, hydroxyl, mercapto, nitro, cyano, oxo, C₁₋₆ alkyl, C₂₋₆ alkenyl, C₂₋₆ alkynyl, C₃₋₁₀ cycloalkyl, C₆₋₁₀ aryl, 5- to 10-membered heteroaryl, a 3- to 10-membered non-aromatic heterocyclic group, C₁₋₆ alkoxy, C₁₋₆ alkylthio, mono-C₁₋₆ alkylamino and di-C₁₋₆ alkylamino.

2. A method of claim 1, wherein R¹ represents a 3- to 10-membered non-aromatic heterocyclic group optionally substituted with a substituent selected from Substituent Group A or Substituent Group B recited in claim 1, wherein the group is limited to a group having nitrogen as a ring constituent atom and the nitrogen having a bonding hand.

3. A method of claim 1, wherein R¹ represents a group represented by the formula (II): 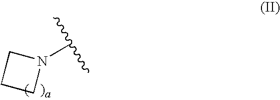
wherein a represents an integer of 1 to 4;
or a group represented by the formula (III): 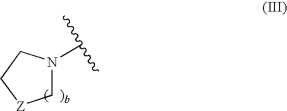
wherein b represents an integer of 1 to 3, and Z represents oxygen, sulfur, carbonyl, sulfonyl, or a group represented by the formula —NR^{Z}—, wherein R^{Z} represents hydrogen or C₁₋₆ alkyl, and the groups represented by the formula (II) or (III) may be substituted with a substituent selected from Substituent Group A or Substituent Group B recited in claim 1.

4. A method of claim 1, wherein R¹ represents azetidin-1-yl optionally substituted with a substituent selected from Substituent Group D, pyrrolidin-1-yl optionally substituted with a substituent selected from Substituent Group D, piperidin-1-yl optionally substituted with a substituent selected from Substituent Group D, azepan-1-yl optionally substituted with a substituent selected from Substituent Group D, piperazin-1-yl optionally substituted with a substituent selected from Substituent Group D, diazepan-1-yl optionally substituted with a substituent selected from Substituent Group D, morpholin-4-yl optionally substituted with a substituent selected from Substituent Group D, thiomorpholin-4-yl optionally substituted with a substituent selected from Substituent Group D, 1,1-dioxothiomorpholin-4-yl optionally substituted with a substituent selected from Substituent Group D,
wherein Substituent Group D consists of halogen, hydroxyl, mercapto, cyano, formyl, oxo, C₁₋₆ alkyl, C₃₋₁₀ cycloalkyl, C₁₋₆ alkoxy, amino, mono-C₁₋₆ alkylamino, di-C₁₋₆ alkylamino, azetidinyl, pyrrolidinyl, piperidinyl, piperazinyl, diazepanyl and a group represented by -T⁴-T⁵, wherein T⁴ represents carbonyl or sulfonyl, and T⁵ represents C₁₋₆ alkyl, C₃ cycloalkyl, azetidinyl, pyrrolidinyl, piperidinyl, hydroxyl, C₁₋₆ alkoxy, amino, mono-C₁₋₆ alkylamino or di-C₁₋₆ alkylamino,
where each group included in Substituent Group D may be substituted with hydroxyl, C₁₋₆ alkyl, di-C₁₋₆ alkylamino, azetidinyl or pyrrolidinyl.

5. A method of claim 1, wherein R¹ represent azetidin-1-yl optionally substituted with a substituent selected from Substituent Group E, pyrrolidin-1-yl optionally substituted with a substituent selected from Substituent Group E, piperidin-1-yl optionally substituted with a substituent selected from Substituent Group E, piperazin-1-yl optionally substituted with a substituent selected from Substituent Group E, diazepan-1-yl optionally substituted with a substituent selected from Substituent Group E or morpholin-4-yl optionally substituted with a substituent selected from Substituent Group E,
wherein Substituent Group E consists of methyl, ethyl, dimethylamino, azetidinyl, pyrrolidinyl, piperidinyl and piperazinyl,
where each group included in Substituent Group E may be substituted with hydroxyl, methyl, dimethylamino, azetidinyl, pyrrolidinyl or piperidinyl.

6. A method of claim 1, wherein R¹ represents azetidin-1-yl optionally substituted with a substituent selected from Substituent Group G, pyrrolidin-1-yl optionally substituted with a substituent selected from Substituent Group G, piperidin-1-yl optionally substituted with a substituent selected from Substituent Group G or piperazin-1-yl optionally substituted with a substituent selected from Substituent Group G,
wherein Substituent Group G consists of dimethylamino, azetidinyl, pyrrolidinyl, piperidinyl, piperazinyl, dimethylaminomethyl, dimethylaminoethyl, azetidin-1-ylmethyl, pyrrolidin-1-ylmethyl and piperidin-1-ylmethyl,
where each group included in Substituent Group G may be substituted with methyl or dimethylamino.

7. A method of claim 1, wherein R¹ represents a group represented by the formula —NR^{11a}R^{11b}, wherein R^{11a} and R^{11b} represent the same meaning as recited in claim 1.

8. A method of claim 1, wherein R¹ represents a group represented by the formula —NR^{11c}R^{11d}, wherein R^{11c} represents hydrogen or C₁₋₆ alkyl, and R^{11d} represents C₁₋₆ alkyl or a group represented by the formula (IV): 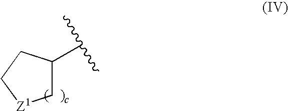
wherein c represents an integer of 1 to 3, and Z¹ represents oxygen, sulfur, carbonyl, sulfonyl or a group represented by the formula —NR^{Z1}—, wherein R^{Z1} represents hydrogen or C₁₋₆ alkyl, and R^{11d} may be substituted with a substituent selected from Substituent Group A or Substituent Group B recited in claim 1.

9. A method of claim 1, wherein R¹ represents a group represented by the formula —NR^{11e}R^{11f}, wherein R^{11e} represents hydrogen or C₁₋₆ alkyl, and R^{11f} represents C₁₋₆ alkyl, pyrrolidin-3-yl, piperidin-3-yl, piperidin-4-yl or tetrahydropyran-4-yl, and R^{11f} may be substituted with a substituent selected from Substituent Group D recited in claim 4.

10. A method of claim 1, wherein R¹ represents a group represented by the formula —NR^{11g}R^{11h}, wherein R^{11g} represents hydrogen or methyl, and R^{11h} represents n-propyl, n-butyl, pyrrolidin-3-yl, piperidin-3-yl, piperidin-4-yl or tetrahydropyran-4-yl, and R^{11h} may be substituted with a substituent selected from Substituent Group F,
wherein Substituent Group F consists of methyl, ethyl, n-propyl, acetyl, dimethylamino, diethylamino, azetidinyl, pyrrolidinyl and piperazinyl,
where each group included in Substituent Group F may be substituted with methyl or dimethylamino.

11. A method of claim 1, wherein R¹ represents a group represented by the formula —N(CH₃)R¹¹ⁱ, wherein R¹¹ⁱ represents n-propyl, n-butyl, pyrrolidin-3-yl or piperidin-4-yl, and R¹¹ⁱ may be substituted with a substituent selected from Substituent Group H,
wherein Substituent Group H consists of dimethylamino, diethylamino, dimethylaminoethyl, dimethylaminopropyl and 1-methylazetidin-3-yl.

12. A method of claim 1, wherein R¹ represents a group represented by the formula N(CH₃)R^{11j}, wherein R^{11j} represents 1-methylpiperidin-4-yl or 1-ethylpiperidin-4-yl.

13. A method of claim 1, wherein R⁴, R⁵, R⁶ and R⁷ may be the same or different and each represents hydrogen, halogen or C₁₋₆ alkyl.

14. A method of claim 1, wherein R⁸ represents hydrogen.

15. A method of claim 1, wherein X represents a group represented by the formula —C(R^{10a})═, wherein R^{10a} represents hydrogen, halogen or cyano.

16. A method of claim 1, wherein X represents nitrogen.

17. A method of claim 1, wherein n represents 1.

18. A method of claim 1, wherein R⁹ represents mono-C₁₋₆ alkylamino optionally substituted with a substituent selected from Substituent Group A or Substituent Group B recited in claim 1, mono-C₃₋₁₀ cycloalkylamino optionally substituted with a substituent selected from Substituent Group A or Substituent Group B recited in claim 1, mono-C₆₋₁₀ arylamino optionally substituted with a substituent selected from Substituent Group A or Substituent Group B recited in claim 1, mono-5- to 10-membered heteroarylamino optionally substituted with a substituent selected from Substituent Group A or Substituent Group B recited in claim 1 or mono-4- to 10-membered non-aromatic heterocyclic amino optionally substituted with a substituent selected from Substituent Group A or Substituent Group B recited in claim 1.

19. A method of claim 1, wherein R⁹ represents mono-C₃₋₁₀ cycloalkylamino optionally substituted with a substituent selected from Substituent Group A or Substituent Group B recited in claim 1 or mono-C₆₋₁₀ arylamino optionally substituted with a substituent selected from Substituent Group A or Substituent Group B recited in claim 1.

20. A method of claim 1, wherein a compound represented by the formula (I) is
(1) N-[4-({2-[({4-[2-(Dimethylamino)ethyl]piperazin-1-yl}carbonyl)amino]pyridin-4-yl}oxy)-2-fluorophenyl]-N′-(4-fluorophenyl)cyclopropane-1,1-dicarboxamide,
(2) N-(2-Fluoro-4-{[2-({[methyl(1-methylpiperidin-4-yl)amino]carbonyl}amino)pyridin-4-yl]oxy}phenyl)-N′-(4-fluorophenyl)cyclopropane-1,1-dicarboxamide,
(3) N-(4-Fluorophenyl)-N′-{2-fluoro-4-[(2-{[(4-pyrrolidin-1-ylpiperidin-1-yl)carbonyl]amino}pyridin-4-yl)oxy]phenyl}cyclopropane-1,1-dicarboxamide,
(4) N-[4-({2-[({4-[(Dimethylamino)methyl]piperidin-1-yl}carbonyl)amino]pyridin-4-yl}oxy)-2-fluorophenyl]-N′-(4-fluorophenyl)cyclopropane-1,1-dicarboxamide,
(5) N-{4-[(2-{[(4-Azetidin-1-ylpiperidin-1-yl)carbonyl]amino}pyridin-4-yl)oxy]-2-fluorophenyl}-N′-(4-fluorophenyl)cyclopropane-1,1-dicarboxamide,
(6) N-[4-({2-[({4-[3-(Dimethylamino)azetidin-1-yl]piperidin-1-yl}carbonyl)amino]pyridin-4-yl}oxy)-2-fluorophenyl]-N′-(4-fluorophenyl)cyclopropane-1,1-dicarboxamide,
(7) N-(2-Fluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide,
(8) N-(2-Fluoro-4-{[2-({[4-(1-methylpiperidin-4-yl)piperazin-1-yl]carbonyl}amino)pyridin-4-yl]oxy}phenyl)-N′-(4-fluorophenyl)cyclopropane-1,1-dicarboxamide,
(9) N-(2-Fluoro-4-{[2-({[4-(1-methylazetidin-3-yl)piperazin-1-yl]carbonyl}amino)pyridin-4-yl]oxy}phenyl)-N′-(4-fluorophenyl)cyclopropane-1,1-dicarboxamide,
(10) N-(4-{[2-({[4-(Dimethylamino)piperidin-1-yl]carbonyl}amino)pyridin-4-yl]oxy}-2-fluorophenyl)-N′-(4-fluorophenyl)cyclopropane-1,1-dicarboxamide,
(11) N-(4-{[2-({[4-(Azetidin-1-ylmethyl)piperidin-1-yl]carbonyl}amino)pyridin-4-yl]oxy}-2-fluorophenyl)-N′-(4-fluorophenyl)cyclopropane-1,1-dicarboxamide,
(12) N-(4-Fluorophenyl)-N′-(2-fluoro-4-{[2-({[4-(pyrrolidin-1-ylmethyl)piperidin-1-yl]carbonyl}amino)pyridin-4-yl]oxy}phenyl)cyclopropane-1,1-dicarboxamide,
(13) N-(4-{[2-({[(3S)-3-(Dimethylamino)pyrrolidin-1-yl]carbonyl}amino)pyridin-4-yl]oxy}-2-fluorophenyl)-N′-(4-fluorophenyl)cyclopropane-1,1-dicarboxamide,
(14) N-(4-{[2-({[(3R)-3-(Dimethylamino)pyrrolidin-1-yl]carbonyl}amino)pyridin-4-yl]oxy}-2-fluorophenyl)-N′-(4-fluorophenyl)cyclopropane-1,1-dicarboxamide,
(15) N-(2-Fluoro-4-{[2-({[methyl(1-methylpiperidin-4-yl)amino]carbonyl}amino)pyridin-4-yl]oxy}phenyl)-N′-phenylcyclopropane-1,1-dicarboxamide,
(16) N-(2-Fluoro-4-{[2-({[4-(4-methylpiperazin-1-yl)piperidin-1-yl]carbonyl}amino)pyridin-4-yl]oxy}phenyl)-N′-phenylcyclopropane-1,1-dicarboxamide,
(17) N-[4-({2-[({4-[3-(Dimethylamino)azetidin-1-yl]piperidin-1-yl}carbonyl)amino]pyridin-4-yl}oxy)-2-fluorophenyl]-N′-phenylcyclopropane-1,1-dicarboxamide,
(18) N-(4-{[2-({[(1-Ethylpiperidin-4-yl)(methyl)amino]carbonyl}amino)pyridin-4-yl]oxy}-2-fluorophenyl)-N′-phenylcyclopropane-1,1-dicarboxamide,
(19) N-[4-({2-[(Azetidin-1-ylcarbonyl)amino]pyridin-4-yl}oxy)-2-fluorophenyl]-N′-(4-fluorophenyl)cyclopropane-1,1-dicarboxamide,
(20) N-(4-Fluorophenyl)-N′-[2-fluoro-4-({2-[(pyrrolidin-1-ylcarbonyl)amino]pyridin-4-yl}oxy)phenyl]cyclopropane-1,1-dicarboxamide,
(21) N-{2-Fluoro-4-[(2-{[(3-hydroxyazetidin-1-yl)carbonyl]amino}pyridin-4-yl)oxy]phenyl}-N′-(4-fluorophenyl)cyclopropane-1,1-dicarboxamide,
(22) N-[4-({2-[(1,3′-Biazetidin-1′-ylcarbonyl)amino]pyridin-4-yl}oxy)-2-fluorophenyl]-N′-(4-fluorophenyl)cyclopropane-1,1-dicarboxamide,
(23) N-(2-Fluoro-4-{[2-({[3-(hydroxymethyl)azetidin-1-yl]carbonyl}amino)pyridin-4-yl]oxy}phenyl)-N′-(4-fluorophenyl)cyclopropane-1,1-dicarboxamide,
(24) N-(4-{[2-({[3-(Dimethylamino)azetidin-1-yl]carbonyl}amino)pyridin-4-yl]oxy}-2-fluorophenyl)-N′-(4-fluorophenyl)cyclopropane-1,1-dicarboxamide,
(25) N-[4-({2-[({3-[(Dimethylamino)methyl]azetidin-1-yl}carbonyl)amino]pyridin-4-yl}oxy)-2-fluorophenyl]-N′-(4-fluorophenyl)cyclopropane-1,1-dicarboxamide,
(26) N-{2-Fluoro-4-[(2-{[(4-hydroxypiperidin-1-yl)carbonyl]amino}pyridin-4-yl)oxy]phenyl}-N′-(4-fluorophenyl)cyclopropane-1,1-dicarboxamide,
(27) N-(2-Fluoro-4-{[2-({[4-(hydroxymethyl)piperidin-1-yl]carbonyl}amino)pyridin-4-yl]oxy}phenyl)-N′-(4-fluorophenyl)cyclopropane-1,1-dicarboxamide,
(28) N-(2-Fluoro-4-{[2-({[(3R)-3-hydroxypyrrolidin-1-yl]carbonyl}amino)pyridin-4-yl]oxy}phenyl)-N′-(4-fluorophenyl)cyclopropane-1, -dicarboxamide,
(29) N-(2-Fluoro-4-{[2-({[(3S)-3-hydroxypyrrolidin-1-yl]carbonyl}amino)pyridin-4-yl]oxy}phenyl)-N′-(4-fluorophenyl)cyclopropane-1,1-dicarboxamide,
(30) N-[4-({2-[(Azetidin-1-ylcarbonyl)amino]pyridin-4-yl}oxy)-2,5-difluorophenyl]-N′-(4-fluorophenyl)cyclopropane-1,1-dicarboxamide,
(31) N-{2,5-Difluoro-4-[(2-{[(3-hydroxyazetidin-1-yl)carbonyl]amino}pyridin-4-yl)oxy]phenyl}-N′-(4-fluorophenyl)cyclopropane-1,1-dicarboxamide,
(32) N-(2,5-Difluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide,
(33) N-[2,5-Difluoro-4-({2-[({3-[(dimethylamino)methyl] azetidin-1-yl}carbonyl)amino]pyridin-4-yl}oxy)phenyl]-N′-(4-fluorophenyl)cyclopropane-1,1-dicarboxamide,
(34) N-(2,5-Difluoro-4-{[2-({[methyl(1-methylpiperidin-4-yl)amino]carbonyl}amino)pyridin-4-yl]oxy}phenyl)-N′-(4-fluorophenyl)cyclopropane-1,1-dicarboxamide,
(35) N-{4-[(2-{[3-(Azetidin-1-ylmethyl)azetidin-1-ylcarbonyl]amino}pyridin-4-yl)oxy]-2,5-difluorophenyl}-N′-(4-fluorophenyl)cyclopropane-1,1-dicarboxamide,
(36) N-(2,5-Difluoro-4-{[2-({[3-(hydroxymethyl)azetidin-1-yl]carbonyl}amino)pyridin-4-yl]oxy}phenyl)-N′-(4-fluorophenyl)cyclopropane-1,1-dicarboxamide,
(37) N-{2,5-Difluoro-4-[(4-{[(3-hydroxyazetidin-1-yl)carbonyl]amino}pyrimidin-6-yl)oxy]phenyl}-N′-(4-fluorophenyl)cyclopropane-1,1-dicarboxamide,
(38) N-[4-({4-[({3-[(Dimethylamino)methyl] azetidin-1-yl}carbonyl)amino]pyrimidin-6-yl}oxy)-2,5-difluorophenyl]-N′-(4-fluorophenyl)cyclopropane-1,1-dicarboxamide,
(39) N-(2,5-Difluoro-4-{[4-({[3-(hydroxymethyl)azetidin-1-yl]carbonyl}amino)pyrimidin-6-yl]oxy}phenyl)-N′-(4-fluorophenyl)cyclopropane-1,1-dicarboxamide,
(40) N-(2,5-Difluoro-4-{[4-({[methyl(1-methylpiperidin-4-yl)amino]carbonyl}amino)pyrimidin-6-yl]oxy}phenyl)-N′-(4-fluorophenyl)cyclopropane-1,1-dicarboxamide,
(41) N-(2,5-Difluoro-4-{[4-({[4-(4-methylpiperazin-1-yl)piperidin-1-yl]carbonyl}amino)pyrimidin-6-yl]oxy}phenyl)-N′-(4-fluorophenyl)cyclopropane-1,1-dicarboxamide,
(42) N-(4-{[2-({[4-(Dimethylamino)piperidin-1-yl]carbonyl}amino)pyridin-4-yl]oxy}-2,5-difluorophenyl)-N′-(4-fluorophenyl)cyclopropane-1,1-dicarboxamide,
(43) N-{2,5-Difluoro-4-[(2-{[(4-methylpiperazin-1-yl)carbonyl]amino}pyridin-4-yl]oxy}phenyl)-N′-(4-fluorophenyl)cyclopropane-1,1-dicarboxamide,
(44) N-{2,5-Difluoro-4-[(2-{[(4-hydroxypiperidin-1-yl)carbonyl]amino}pyridin-4-yl)oxy]phenyl}-N′-(4-fluorophenyl)cyclopropane-1,1-dicarboxamide,
(45) N-{4-[(2-{[(4-Azetidin-1-ylpiperidin-1-yl)carbonyl]amino}pyridin-4-yl)oxy]oxy}-2,5-difluorophenyl}-N′-(4-fluorophenyl)cyclopropane-1,1-dicarboxamide,
(46) N-(2,5-Difluoro-4-{[2-({[3-(2-dimethylaminoacetoxy)azetidin-1-yl]carbonyl}amino)pyridin-4-yl]oxy}phenyl)-N′-(4-fluorophenyl)cyclopropane-1,1-dicarboxamide,
(47) N-(2,5-Difluoro-4-{[2-({[(3S)-3-hydroxypyrrolidin-1-yl]carbonyl}amino)pyridin-4-yl]oxy}phenyl)-N′-(4-fluorophenyl)cyclopropane-1,1-dicarboxamide or
(48) N-(2,5-Difluoro-4-{[2-({[(3R)-3-hydroxypyrrolidin-1-yl]carbonyl}amino)pyridin-4-yl]oxy}phenyl)-N′-(4-fluorophenyl)cyclopropane-1,1-dicarboxamide.

21. A method of claim 1, wherein a compound represented by the formula (I) is
(1) N-(2-Fluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide
(2) N-[4-({2-[(Azetidin-1-ylcarbonyl)amino]pyridin-4-yl}oxy)-2-fluorophenyl]-N′-(4-fluorophenyl)cyclopropane-1,1-dicarboxamide
(3) N-{2,5-Difluoro-4-[(2-{[(3-hydroxyazetidin-1-yl)carbonyl]amino}pyridin-4-yl)oxy]phenyl}-N′-(4-fluorophenyl)cyclopropane-1,1-dicarboxamide
(4) N-(2,5-Difluoro-4-{[2-({[4-(4-methylpiperazin-1-yl)piperidin-1-yl]carbonyl}amino)pyridin-4-yl]oxy}phenyl)-N′-(4-fluorophenyl)cyclopropane-1,1-dicarboxamide
(5) N-(2,5-Difluoro-4-{[2-({[methyl(1-methylpiperidin-4-yl)amino]carbonyl}amino)pyridin-4-yl]oxy}phenyl)-N′-(4-fluorophenyl)cyclopropane-1,1-dicarboxamide or
(6) N-(2,5-Difluoro-4-{[2-({[3-(hydroxymethyl)azetidin-1-yl]carbonyl}amino)pyridin-4-yl]oxy}phenyl)-N′-(4-fluorophenyl)cyclopropane-1,1-dicarboxamide.
